(19) United States
(12) Patent Application Publication
(10) Pub. No.: US 2015/0057162 A1
(43) Pub. Date: Feb. 26, 2015

(54) PEPTIDE ARRAYS

(71) Applicant: Isogenica LTD., Little Chesterford,Essex (GB)

(72) Inventors: Christopher Ullman, Little Chesterford (GB); Neil Cooley, Little Chesterford (GB); Laura Frigotto, Little Chesterford (GB); Pascale Mathonet, Little Chesterford (GB); Nahida Parveen, Little Chesterford (GB)

(73) Assignee: ISOGENICA LTD., Little Chesterford, Essex (GB)

(21) Appl. No.: 14/385,190

(22) PCT Filed: Mar. 15, 2013
(86) PCT No.: PCT/GB2013/050676

§ 371 Date: Sep. 15, 2014

(30) Foreign Application Priority Data
Mar. 15, 2012 (GB) 1204605.8

(51) Int. Cl. G01N 33/68

(52) U.S. Cl. CPC G01N 33/6845; G01N 2570/00
USPC 506/2

(57) ABSTRACT

A method is disclosed for identifying a member of a peptide library that interacts with a target molecule in situ, the method including expressing immobilised nucleic acid molecules to produce the peptide library in a way that each member of the peptide library is immobilised on the nucleic acid molecule from which it was expressed; contacting the immobilised peptide library with the target molecule; and detecting an interaction between at least one member of the peptide library and the target molecule. The method further comprises sequencing the plurality of nucleic acid molecules in situ on the solid support, such that the at least one member of the peptide library that interacts with the target molecule can be immediately identified, at least by the sequence of the nucleic acid molecule from which it was expressed, without requiring additional or secondary analysis or characterising procedures in order to identify the useful members of the library. The target molecules may themselves be comprised within a second nucleic acid or peptide library.

### FIELD OF THE INVENTION

This invention relates to methods for peptide screening and sequencing. In particular, the invention relates to in situ sequencing of a nucleic acid encoding a peptide and screening of the peptide to identify a desirable activity or property. The methods are particularly suitable for the parallel sequencing and expression of immobilised nucleic acids in a nucleic acid library, and screening of the expressed peptide libraries to identify and characterise individual peptides of known sequence having desirable properties.

### BACKGROUND OF THE INVENTION

Genomic sequencing has enabled researchers to understand the natural DNA code that is contained within our cells. The drive towards generating higher throughput for less cost has resulted in the development of different techniques to the sequencing methods originally invented by Sanger and Gilbert. This progress has been assisted by a range of advances in fields such as microscopy, surface chemistry, fluorophores, microfluidics, polymerase engineering, library preparation and parallel methods for template extension.

Until recently, parallel methods for DNA sequencing were limited to semi-automated capillary-based implementations of Sanger biochemistry, normally restricted to between 96 and 384 parallel reactions. However, more recently ‘second-generation’ or ‘next-generation’ techniques have emerged. These are dominated by cyclic-array sequencing methods, some of which are now commercially available: such as 454 sequencing, Illumina sequencing, SOLiD™ sequencing platform, Polonator, Ion Torrent and HeliScope Single Molecule Sequencer technologies. The fundamental principle behind cyclic-array methodologies is the sequencing of a DNA array through iterative cycles of enzymatic processing and image-based data collection.

Typically, the initial library is prepared by random fragmentation of the DNA or by ligation of adaptor sequences. The next step is to amplify the sequences in a manner to produce a clonally clustered population which is discretely separated from other clusters on a planar surface or on the surface of micro-beads. The clonal amplification may be achieved by in situ polonies (polymerase colonies), bridge polymerase chain reaction (bridge-PCR), or emulsion-PCR. Emulsion-PCR is performed on DNA immobilised on beads, whereas the former techniques are practiced on a planar substrate such as a glass slide.

Some of the latest generations of sequencing technologies allow sequencing in ‘real time’, for instance, where nucleic acids are passed through a pore and the change in conductance in relation to the DNA sequence is measured (nanopore). For a review of second and third generation sequencing techniques see e.g. Gupta (2008), Trends Biotechnol., 26(11), 602-611; Shendure & Li (2008), Nature Biotechnol., 26(10), 1135-1145; and Pettersson et al., (2009), Genomics, 93, 105-111. Another real time sequencing technology is a process that determines the base incorporated by the polymerase using a fluorescently labelled enzyme and gamma-phosphate-labelled nucleotides in a FRET (fluorescent resonance energy transfer) based approach (e.g. Pacific).

However, despite progress in the sequencing of DNA through array approaches, screening of protein or peptide populations has not matched the density of the DNA arrays. In addition, in the prior art it is not possible to simultaneously/in parallel determine the sequence of a peptide and its ability to bind a target molecule using the same array. In order to extract the most useful information from a peptide array screen, i.e. to enable an observed peptide phenotype (such as a binding interaction) to be correlated back to its sequence, the prior art procedures require either: (i) that the sequence of the peptide or protein is known prior to manufacturing the array, and that a predetermined peptide or its encoding nucleic acid is placed in a specific location of an array; or (ii) that the sequence of any clones (peptides or their encoding nucleic acids) are determined in a separate DNA sequencing assay (e.g. via PCR or RT-PCR) following the identification of a desirable peptide attribute. Therefore, in these approaches there is either a priori knowledge of the peptide or protein sequence, or it is obtained at a later time through sequencing of the individual clone. In either case, the determination of encoding nucleic acid sequence (and thus the sequence of the peptide) is decoupled from phenotype selection (e.g. the peptide's ligand binding abilities). These limitations mean that there is a cost associated with the synthesis of each individual peptide, or in identifying the peptide sequence post hoc. As the size and complexity of the peptide arrays increase, so does the total cost. This is at least one reason why peptide arrays have, to date, not matched the equivalent nucleic acid arrays for size, complexity and information outputs.

Examples of the peptide array prior art include: WO2006/131687 where the proteins are arrayed onto a different surface than the nucleic acid in an ordered array; where proteins are produced from immobilised DNA templates but sequence determination is not envisaged and the protein is tethered onto the array through a tag capture (WO02/14860); or an immobilised antibody (WO 02/059601) onto the surface and not through direct binding to its own nucleic acid template (see also Darmanis et al. (2011), PLoS One, 6, e25583); and WO2007/047850 where a specific DNA binding protein is used to immobilise a fusion protein. However, in all these teachings a priori knowledge of the placement of the clone is necessary. In US2011/0287945, it is recognised that a next generation sequencing machine contains the necessary components (i.e. microfluidics and sensitive detection apparatus) for the determination of molecular interactions, however, it was not envisaged that a protein may be synthesised from its own DNA and would be able to tether its very own coding sequence, such that the coding sequence could be determined by sequencing, and the function or binding properties of that protein encoded by the DNA determined in the same array without prior knowledge of either the DNA, or the protein sequence, or a predetermined arrangement of the array and its components.

Accordingly, there is a need in the art for more effective and efficient systems that can utilise devices for DNA arrays in order to deconvolute sequence, binding and functional properties of proteins in the same arrays through coupling the desirable phenotype/property of a peptide or nucleic acid in a library with its nucleic acid sequence.

The present invention seeks to overcome or at least alleviate one or more of the problems in the prior art.

### SUMMARY OF THE INVENTION

In general terms, the present invention provides a system in which both the sequencing and the binding or activity characteristics of a polyclonal nucleic acid or peptide population are determined in situ. The nucleic acid molecules of the polyclonal population may be immobilised such that the nucleic acid (DNA) sequence of a library member may be determined in exactly the same position (e.g. of an array) as that in which it is screened for a desirable phenotype: for example, a binding interaction between an expressed peptide and a target molecule. In this way, one or more phenotypes of a peptide or nucleic acid may be determined in situ from the same library display; or different peptides or nucleic acids may be identified and characterised from the same library using different selection criteria in sequential procedures.

The selection procedure may be based on an in vitro selection system. One convenient approach employs a method of displaying proteins attached to their own DNA sequence on a next generation sequencing platform.

Useful sequencing methods involve, but are not limited to hybridisation of single-stranded DNA on beads (e.g. using emulsion-PCR) or on a planar surface, followed by sequencing using pyrosequencing, HeliScope, Illumina, nanopore sequencing, SOLiD™ or Ion torrent processes and the like. The appropriate methods for DNA sequencing in this invention maintain the integrity of at least one strand of the DNA template so that corresponding double-stranded DNA can be recreated (e.g. using a suitable polymerase), and the DNA can then be further manipulated: for example, it may be transcribed and translated into the peptide that it encodes for peptide screening and/or selection. Of course, the invention is also useful for screening libraries of nucleic acids for one or more desirable property of a nucleic acid (e.g. nucleic acid binding or inhibitor molecules).

Thus, in one aspect of the invention there is provided a method for identifying a member of a peptide library that interacts with a target molecule in situ, the method comprising: (a) providing a plurality of nucleic acid molecules each encoding a member of the peptide library; (b) immobilising the plurality of nucleic acid molecules on a solid support; (c) sequencing the plurality of nucleic acid molecules in situ on the solid support; (d) expressing the immobilised nucleic acid molecules to produce the peptide library, wherein each member of the peptide library is immobilised on the nucleic acid molecule from which it was expressed; (e) contacting members of the immobilised peptide library with the target molecule; (f) detecting an interaction between at least one member of the peptide library and the target molecule; and (g) identifying the at least one member of the peptide library that interacts with the target molecule by the sequence of the nucleic acid molecule from which it was expressed.

In another aspect of the invention the method for identifying a member of a peptide library that interacts with a target molecule may be adjusted such that the peptide library is expressed from the plurality of nucleic acid molecules before the nucleic acid molecules are immobilised on a solid support, such that step (d) is performed between steps (a) and (b), and step (c) is performed between steps (f) and (g). Accordingly, in this aspect, the method comprises: (a″) providing a plurality of nucleic acid molecules each encoding a member of the peptide library; (ad) expressing the plurality of nucleic acid molecules to produce the peptide library, wherein each member of the peptide library is immobilised on the nucleic acid molecule from which it was expressed; (b″) immobilising the plurality of nucleic acid molecules having peptides immobilised thereon, on a solid support; (e″) contacting members of the immobilised peptide library with the target molecule; (f″) detecting an interaction between at least one member of the peptide library and the target molecule; (fc) sequencing in situ on the solid support at least the nucleic acid of the plurality of nucleic acid molecules that encoded the at least one member of the peptide library detected in step (f″); and (g″) identifying the at least one member of the peptide library that interacts with the target molecule by the sequence of the nucleic acid molecule from which it was expressed. Thus, according to this aspect, one or more of the plurality of nucleic acids is sequenced. In some embodiments all of the plurality of nucleic acids is sequenced.

The method of the invention is particularly suitable for use with naïve libraries that have not previously been exposed to a target molecule and which have not been previously enriched for potential interacting/binding members. Thus, the method of the invention advantageously does not require multiple cycles of peptide expression, screening and/or selection. Accordingly, in another aspect the invention provides a method for characterising a peptide from a naïve peptide library that interacts with a target molecule, without pre-enrichment of library members, the method comprising: (a) providing a plurality of nucleic acid molecules encoding the naïve peptide library; (b) immobilising the plurality of nucleic acid molecules on a solid support; (c) sequencing the plurality of nucleic acid molecules in situ on the solid support; (d) expressing a plurality of the immobilised nucleic acids to produce the naïve peptide library, wherein peptides are immobilised on the nucleic acid molecules from which they were expressed; (e) contacting the immobilised peptides with the target molecule; (f) detecting an interaction between at least one member of the naïve peptide library and the target molecule; and (g) characterising the at least one member of the naive peptide library that interacts with the target molecule by the sequence of the nucleic acid molecule from which it was expressed; wherein the naïve peptide library has not previously been exposed to the target molecule. As indicated above, this method of the invention may alternatively be performed by expressing peptides from the plurality of nucleic acid molecules before the nucleic acid molecules are immobilised on a solid support, such that step (d) is performed between steps (a) and (b), and, in this embodiment, step (c) is performed between steps (f) and (g).

Furthermore, it will be appreciated that where any step of the methods is not dependent on the order of the preceding steps, then the methods of the invention may be performed in any other suitable order. Thus, the methods of the above aspects may be performed in the order (a) to (g), or may be carried out in the order: (a), (b), (d), (e), (f), (c), (g), or (a), (d), (b), (e), (f), (c), (g), for example.

Members of the peptide library, once expressed, may bind covalently or non-covalently to the nucleic acid molecule from which it was expressed.

Suitably, each of the plurality of nucleic acid molecules comprises: (I) a nucleic acid anchoring sequence; (II) a nucleic acid sequence encoding a member of the peptide library; and (III) a nucleic acid sequence encoding a protein or protein fragment capable of interacting with the nucleic acid anchoring sequence (I). The nucleic acid anchoring sequence (I) advantageously comprises a DNA element that directs cis-activity. The protein or protein fragment capable of interacting with the nucleic acid anchoring sequence of (I) encoded by the nucleic acid sequence of (III) may suitably comprise a sequence of the A protein or the RepA replication initiator protein. In one particularly beneficial embodiment the nucleic acid sequences of (II) and (III) are arranged so as to encode a fusion protein comprising the member of the peptide library and the protein or protein fragment capable of interacting with the nucleic acid anchoring sequence of (I). For example, the nucleic acid anchoring sequence of (I) may comprise a nuclear hormone receptor target sequence, and the protein or protein fragment may comprise a nuclear hormone receptor nucleic acid binding portion. Alternatively, the nucleic acid target sequence of (I) may comprise an E. coli Ter sequence, and the protein or protein fragment may comprise at least a fragment of the E. coli Tus protein.

In other embodiments, each member of the peptide library may bind indirectly to the nucleic acid molecule from which it was expressed via a coupling agent. For example, the nucleic acid anchoring sequence of (I) may comprise a tag or linker capable of being bound by the coupling agent. Such a tag or linker may be selected from biotin and fluorescein. Alternatively, the coupling agent may comprise an antibody or fragment thereof, or a polymer. Suitable polymers may include protein scaffolds, non-protein scaffolds and DNA; and also include polypeptides, polynucleic acids, sugars, or organic molecules, provided they can be used to couple a peptide directly to the nucleic acid that encodes it. This includes cross linking agents that may act to couple the peptide to the nucleic acid molecule from which it was expressed, or puromycin which can covalently link the peptide to the nucleic acid. The nucleic acid molecule encoding the peptide and from which the peptide is expressed may be considered to be a DNA molecule (which is first transcribed into RNA), or may be an RNA molecule.

Each nucleic acid molecule that encodes a member of the peptide library preferably comprises suitable promoter and translation sequences to allow for in vitro transcription and translation of the members of the peptide library. Thus, expressing the plurality of nucleic acid molecules to produce the peptide library in step (d) may comprise contacting the immobilised nucleic acid molecules with a protein expression system capable of directing transcription and translation of the nucleic acid molecules in vitro. Exemplary expression systems include bacterial coupled transcription and translation systems, such as an E. coli S30 extract systems, systems containing SP6, T3 or T7 RNA polymerase, reconstituted component system (such as the PureSystem, Gene Frontier Corporation), or eukaryotic transcription and translation system, such as a rabbit reticulocyte extract, insect cell, wheat germ extract or human cell extract systems.

In some embodiments, step (b) or step (c) may be followed by: providing a double-stranded nucleic acid portion of each of the plurality of nucleic acid molecules in at least the portion of nucleic acid molecule that encodes a member of the peptide library; and/or providing a double-stranded nucleic acid sequence portion attached to each of the plurality of nucleic acid molecules, said double-stranded nucleic acid sequence portion encoding a protein or protein fragment capable of interacting with the nucleic acid molecule that encodes the member of the peptide library to which it is attached.

In another aspect of the invention there is provided a method for obtaining a peptide that interacts with a target molecule, the method comprising: (h) performing the method of any of the above aspects and embodiments of the invention to identify the nucleic acid sequence encoding the at least one member of step (f); (i) obtaining a nucleic acid expression construct encoding the nucleic acid sequence encoding the at least one member of step (f); and (j) expressing the nucleic acid expression construct of (i) to obtain the peptide; optionally further comprising (k) purifying the peptide.

In some embodiments of the inventive method, the target molecule may be a member of a peptide or nucleic acid library, or may be a small (inorganic) molecule coupled to a nucleic acid, such as a DNA tarcode′, e.g. as described in Buller et al., (2010) “High-throughput sequencing for the identification of binding molecules from DNA-encoded chemical libraries”. Bioorg. Med. Chem. Lett., July 15, 20(14): 4188-92. For example, the target molecule may conveniently be expressed from a library of nucleic acid molecules comprising a plurality of unique nucleic acid sequences. Accordingly, in one embodiment, step (e) comprises the steps: (e1) providing a plurality of unique nucleic acid molecules each encoding a potential peptide target molecule; (e2) expressing the plurality of unique nucleic acid molecules to produce a plurality of potential target molecules, wherein each potential target molecule is immobilised on the nucleic acid molecule from which it was expressed; and (e3) contacting the immobilised peptide library of step (d) with the plurality of potential target molecules of step (e2) to detect an interaction between at least one member of the immobilised peptide library and at least one of the plurality of potential target molecules in step (f). Beneficially, the method may further comprise: (e4) identifying the at least one target molecule that interacts with the at least one member of the immobilised peptide library.

In yet another aspect of the invention there is provided a method for identifying a de novo binding partner interaction from a plurality of nucleic acid libraries, the method comprising: (a′) providing a first nucleic acid library comprising a plurality of nucleic acid molecules each encoding a member of a first peptide library (Library 1); (b′) immobilising the plurality of nucleic acid molecules of the first nucleic acid library on a solid support; (c′) sequencing the plurality of nucleic acid molecules of the first nucleic acid library in situ on the solid support; (d′) expressing the immobilised nucleic acid molecules to produce the first peptide library (Library 1), wherein each member of the first peptide library is immobilised on the nucleic acid molecule from which it was expressed; (e′) contacting the immobilised first peptide library (Library 1) with a second library comprising a plurality of nucleic acid molecules; (f′) detecting an interaction between at least one member of the first peptide library (Library 1) and at least one target molecule provided within the second library; (g′) identifying the at least one member of the first peptide library (Library 1) that interacts with the at least one target molecule at least by the sequence of the nucleic acid molecule from which it was expressed; and (h′) identifying the at least one target molecule that interacts with the at least one member of the first peptide library of step (g′). In such methods, step (h′) may optionally be carried out before step (g′). Also, the method of this aspect may be carried out in the order: (a′), (b′), (d′), (e′), (f′), (c′), (g′) and (h′), or in the order: (a′), (b′), (d′), (e′), (f′), (h′), (c′) and (g′), as desired. The method of this aspect may further comprise a step between steps (f′) and (h′) of: (fh′) collecting a peptide-target molecule complex comprising a member of the first peptide library (Library 1) and at least one member of the second library (Library 2) with which it interacts.

In a preferred embodiment, the second library comprises a second peptide library (Library 2). According to such embodiments of the invention, the target molecule within the second peptide library (Library 2) may be provided by: (A) providing a second plurality of nucleic acid molecules each encoding a member of the second peptide library (Library 2); and (B) expressing the second plurality of nucleic acid molecules to produce the second peptide library (Library 2), wherein each member of the peptide library is a potential target molecule and is immobilised on the nucleic acid molecule from which it was expressed.

In any of the aspects and embodiment of the invention, the step of detecting an interaction between at least one member of the peptide library and the target molecule may be performed by fluorescence measurement.

Likewise, in any of the aspects and embodiment of the invention, the step of sequencing the plurality of nucleic acid molecules on the solid support may be performed by a second-generation or next-generation sequencing method, such as ‘sequencing by synthesis’ or ‘single molecule sequencing’. Suitable sequencing processes include 454 sequencing, Illumina sequencing, SOLiD™ sequencing, Polonator sequencing, Ion Torrent sequencing and HeliScope Single Molecule sequencing.

In any of the aspects and embodiments of the invention, the step of immobilising the plurality of nucleic acid molecules on a solid support may be performed by emulsion PCR or bridge PCR. Advantageously, each of the plurality of nucleic acid molecules of the library comprises at least one strand capable of interacting with the solid support so as to immobilise the nucleic acid thereon.

In some particularly suitable aspects and embodiments of the invention, step (c) or step (c′) comprises: (c1) providing an at least partially single-stranded nucleic acid molecule immobilised on the surface of the solid support; (c2) annealing a nucleic acid sequencing primer to a single-stranded portion of the nucleic acid molecule of (c1) to create a partially double-stranded nucleic acid molecule in a region spaced from the sequence encoding the member of the peptide library; (c3) extending the sequencing primer by incorporating nucleic acids by complementary base-pairing to the at least partially single-stranded nucleic acid molecule to produce a double-stranded nucleic acid molecule in at least a region encoding the member of the peptide library; and (c4) detecting the order of nucleic acids incorporated in step (c3) to determine the nucleic acid sequence of the region encoding the member of the peptide library.

A key aspect of this invention is, therefore, that the screening and/or selection (e.g. phenotype) assay is carried out on library members (nucleic acids or peptides) that are immobilised, so that the nucleic acid sequence can be determined in situ and that the sequence can be used directly to characterise any nucleic acid or peptide library member that has been identified in the screening and/or selection assay. When the library screening and/or selection protocol is based on expressed peptides, the peptides to be assayed are beneficially linked to a nucleic acid (DNA) binding protein that is capable of binding back to its very own DNA template from which it was transcribed. Such proteins that bind to their own DNA sequences are known as cis-acting proteins (CAPs) and are characterised, for example, in the publications of Lindqvist (WO98/37186) and Odegrip (WO2004/022746). Two suitable such proteins are the A protein from P2 phage (P2A), and the RepA replication initiator protein from the R1/R100 plasmid, which link covalently or non-covalently, respectively, back to binding regions within their own coding DNA sequence. It is also envisaged that other systems can be used to similar effect, including DNA display methodologies and ribosome display methodologies that link the phenotype to the genotype (e.g. Mattheakis et al., (1994) PNAS, 91, 9022-9026; Hanes and Pluckthun (1997) PNAS, 94, 4937-4942; He and Taussig (1997) NAR, 25, 5132-5134; Nemoto et al., (1997) FEBS Lett. 414, 405-408; Robers and Szostak, (1997) PNAS, 94, 12297-12302; Tawfik & Griffiths, (1998) Nat. Biotech., 16, 652-656; Odegrip et al., (2004) PNAS, 101, 2806-2810; Reiersen et al., (2005) NAR, 33 e10; Bertschinger et al., (2007) Protein. Eng. Des. Sel., 20, 57-68; and in patent applications WO1998/031700; WO1998/016636; WO1998/048008; WO1995/011922; W02011/0183863; and WO2004/022746 and as reviewed by Ullman et al., (2011) Brief Funct. Genomics, 10, 125-134). Thus, in another embodiment, an RNA template may be used which can be translated to express a peptide, and the ribosome stalled and tethered to the nucleic acid to display the expressed peptide (e.g. ‘ribosome display’ or ‘polysome display’). Alternatively, the peptide may be covalently linked to the RNA, DNA or hybrid RNA/DNA molecule through puromycin and/or a linker. The display step may be either prior to or following a sequencing procedure to determine the sequence of each displayed peptide or even prior to immobilisation on the solid support. In other aspects and embodiments, the pre-formed nucleic acid peptide complex or fusion may be annealed to single stranded nucleic acids that have been immobilised on a solid support. The immobilised peptide library may then be contacted with the target molecule, followed by detecting of an interaction between at least one member of the peptide library and the target molecule. Finally, one or more (e.g. all) of the immobilised plurality of nucleic acids may then be sequenced in situ on the solid support. Any (i.e. one or more) members of the immobilised peptide library that interacts with the target molecule may then be identified at least by the sequence of the nucleic acid molecule from which it was expressed.

The invention may further comprise the sequencing and/or synthesis of RNA templates, which are then subsequently used as a template for translation so that the ribosomes are stalled on the RNA template or the expressed protein is attached to the ribosome, RNA or a DNA strand derived from that RNA species, such as in mRNA display (as reviewed by Douthwaite & Jackson, “Ribosome Display and Related Technologies” Edited by Douthwaite & Jackson, 2012, Methods in Molecular Biology, Volume 805, Springer Press), or as described in W02011/0183863 via the action of puromycin, pyrazolopyrimidine, streptavidin-biotin linkage or any other linker. It is also envisaged that macrocycles may also be tethered to the DNA for use in arrays. Such methods of attachment are described in patent application WO02/074929.

The selection and/or screening procedure can be carried out before or after the nucleic acid sequencing procedure, once the nucleic acids have been immobilised in a suitable format. Conveniently, the immobilised DNA molecules are subjected to transcription and translation following sequencing of the nucleic acid. Generally, the sequencing procedure is carried out on single-stranded, substantially single-stranded or partially single-stranded nucleic acid molecules, and so when sequencing is carried out prior to screening, the double-stranded DNA template must generally be rebuilt prior to transcription and translation.

In one suitable embodiment, a peptide-CAP fusion protein is generated that spontaneously binds back to its own DNA sequence, through the CAP recognising its own binding sequence on its own template. As a result, the peptide is advantageously displayed on its own coding DNA molecule in exactly the same position (e.g. of an array) as its immobilised encoding DNA molecule. Typically, the expressed peptide is thus non-covalently attached (immobilised′, ‘tethered’ or ‘anchored’) to its encoding DNA and is available for a screening and/or selection process. In other embodiments the CAP is bound covalently to its encoding nucleic acid template to achieve the same effect.

In some preferred embodiments, the expressed, immobilised peptides are screened for their ability to bind to a target molecule—thus, the desirable property or characteristic may be binding affinity or specificity to a target molecule. Where a library of peptides is displayed then all of the peptides that are competent for binding to a particular target molecule can be detected individually. This can provide a significant advantage over existing selection/screening methodologies, in which a mixed population of active members will result.

Desirably, the detection of a binding event or activity in the screening/selection protocol utilises the same technology (e.g. chemistry) as used for sequence determination: for example, a FRET-based system using a fluorescently labelled protein and a labelled target; through fluorescence detection of a fluorescently labelled target; or through an enzyme-linked approach (e.g. which causes the depletion of a hydrogen ion). This advantageously alleviates the need for a different array or detection apparatus to be used in the method of the invention and provides yet further simplicity, convenience, economies and efficiencies.

Beneficially, the immobilised nucleic acid library members are immobilised in an ‘array’. The array is conveniently ordered, e.g. in the form of a grid. Accordingly, in a particularly suitable embodiment, positive signals generated in the screening and/or selection process (e.g. as a result of a peptide-target molecule binding interaction) can be detected in exactly the same place of an array following the sequencing reaction and will, therefore, provide a means to determine the DNA sequence of the arrayed clones, and also the capacity of the protein encoded by the DNA to bind one or more target molecules presented to the array. In this way the process analyses and provides sequence and binding data in a single array and in an in situ parallel assay for a population of nucleic acid molecules. The array may also be of random nature in which the nucleic acid molecules hybridise randomly to the prepared surface of the slide. In such a random system, bridge PCR amplification would then create clusters of identical nucleic acids immobilised randomly to the surface.

In another aspect the invention relates to release of binding molecules and their associated DNA from the array through cleavage of a photocleavable linker within the DNA sequence by the action of a beam of light focused upon a spot on the array or upon a bead immobilised on the array. Alternatively, magnetic beads may be specifically released from the array via the action of electromagnetic release or an electrical stimulus or through some other suitable means, such as being lifted or forced out of a well of an array by a pressure difference or, again, by the action of magnets.

It will be appreciated that peptides of the invention may be further derivatised or conjugated to additional molecules, and that such peptide derivatives and conjugates fall within the scope of the invention. It is also envisaged that modified nucleic acids may be used or ligated to the immobilised nucleic acid regions for further binding analysis.

The invention also encompasses therapeutic and diagnostic uses for the novel peptides identified by the methods of the invention having desirable properties. Aspects and embodiments of the invention thus include formulations, medicaments and pharmaceutical compositions comprising the peptides and derivatives thereof according to the invention. In one embodiment the invention relates to a peptide or its derivative for use in medicine. More specifically, for use in antagonising or agonising the function of a target ligand, such as a cell-surface receptor. The peptides of the invention may be used in the treatment of various diseases and conditions of the human or animal body, such as cancer, and degenerative diseases. Treatment may also include preventative as well as therapeutic treatments and alleviation of a disease or condition. Accordingly, the present invention further encompasses methods for the selection and identification of therapeutic peptides using the methods described herein.

The invention also has application in the identification of biomarkers, for example, the method may comprise expression of disease epitopes derived from mRNA species and cloning cDNA extracted from patient tissues; displaying and expressing these cDNAs on the surface of the array; and detecting or recognising antibodies (e.g. antibodies from within the patient) that might distinguish unusual epitopes in disease tissues (e.g. epitopes that are not expressed in normal tissues). Thus, the method may involve comparing the output of the above test with a comparison based on expression of cDNAs from a healthy tissue or patient. Disease-specific epitopes can be used to diagnose the presence or severity of disease conditions. Used in this way the epitopes discovered by the methods described herein can be used as reagents and in kits for disease diagnostics. Likewise, the invention has utility in vaccine research by recognition of epitopes within infectious agents by arraying libraries of DNA or RNA extracted from microorganisms or viruses/virus infected cells expressing the proteins and displaying these in the array, followed by identification of a binding and neutralising molecule by passing a library of proteins or antibodies attached to their coding sequence over the array, or vice versa. In addition, the invention also allows the analysis of chromatin-binding proteins by expressing cDNA on the surface of the array and passing genomic DNA fragments over the array which may then be captured by a chromatin-binding protein expressed on the array. These DNA fragments can then be subsequently released and identified as described elsewhere herein. This approach differs from the current ChIP-seq analysis method (Johnson et al., 2006, Science, 316, 1497-1502; Marioni et al., (2008), “RNA-seq: an assessment of technical reproducibility and comparison with gene expression arrays”. Genome Res., September; 18(9):1509-17).

The invention further encompasses nucleic acids, such as expression vectors, that encode the peptides of the invention and/or the modified peptides or derivatives of the invention. In addition, the invention encompasses the peptides obtainable by the methods of the invention and isolated peptides and nucleic acids.

It should also be appreciated that, unless otherwise stated, optional features of one or more aspects or embodiments of the invention may be incorporated into any other aspect or embodiment of the invention and that all such variations are encompassed within the scope of the invention.

All references cited herein are incorporated by reference in their entirety. Unless otherwise defined, all technical and scientific terms used herein have the same meaning as commonly understood by one of ordinary skill in the art to which this invention belongs.

### BRIEF DESCRIPTION OF THE DRAWINGS

The invention is further illustrated by the accompanying drawings in which:

FIG. 1 illustrates the results of an ELISA assay for the binding of Ck peptides fused to RepA that are produced from an immobilised template (solid phase′) and bound to its own template (left-hand column); or from a template that is not immobilised at the time of transcription/translation and is subsequently attached to a solid surface following transcription/translation (in solution′; right-hand column). The ELISA signal is proportional to the amount of protein immobilised upon the DNA bound to the surface.

FIG. 2 shows the results of an ELISA assay for the binding of V5 peptides fused to RepA that are produced and bound to their own template immobilised on a bead biotinylated at the 3′ end of the DNA template (column 415-514), the 5′ end of the DNA template (column 472-85), or a negative control that was non-biotinylated (column 144-85).

FIG. 3 shows an approach for synthesising proteins from DNA template immobilised on a planar surface following sequencing via Illumina methodology. (A) The DNA template is immobilised by hybridisation onto immobilised oligonucleotides on a planar surface. (B) The immobilised oligonucleotide primes the synthesis of the complementary strand that anneals to an immobilised primer that is complementary to the opposite end of the DNA molecule. (C and D) The second strand is synthesised by primer extension. (E) The double-stranded DNA is then denatured in preparation for sequencing. (F) The double-stranded region encoding the peptide library portion of the template is remade (after sequencing) with polymerase and then cleaved (digested) with a restriction enzyme to provide a free end for ligation. (G) Any template nucleic acid portions common to all library members (e.g. CAP-encoding and tethering sequences, such as the repA-CIS-ori sequence—see Examples) can then be attached to the digested library portions (e.g. the common template portion can be similarly digested and then ligated to the immobilised template portion. (H) An in vitro transcription/translation reaction performed to produce the peptide-CAP-DNA complex which creates a fusion protein comprising the library peptide member bound to its own encoding DNA template molecule through the interaction of the CAP or other coupling mechanism (e.g. RepA via the on Sequence). (I) The expressed peptide can then be detected by any suitable mechanism, such as the specific binding of a protein (e.g. a fluorescently labelled antibody or an antibody conjugated to an enzyme that can be used with a fluorescent reagent).

FIG. 4 demonstrates a variation of the bridge amplification protocol where the full-length construct can be used for expression and display by dilution of the hybridisation oligonucleotides so that discrete clusters of templates can be formed. The DNA template is prepared for sequencing as shown in panels (A) to (E). The appropriate regions of the single-stranded molecules are sequenced and the templates are then denatured, followed by a fill-in reaction to remake the full double-stranded molecule. An in vitro transcription/translation reaction is performed to produce the peptide-CAP DNA complex which creates a fusion protein comprising the library peptide member bound to its own encoding DNA template molecule through the interaction of the CAP or other coupling/anchoring mechanism, as shown in (F). Finally, the expressed peptide can then be detected by any suitable mechanism, such as the specific binding of a protein (e.g. a fluorescently labelled antibody or an antibody conjugated to an enzyme that can be used with a fluorescent reagent), as shown in (G).

FIG. 5 demonstrates a further variation of the bridge amplification protocol where peptide-nucleic acid complexes are prepared by performing an in vitro transcription/translation reaction free in solution, as shown in (A). The peptide-nucleic acid complex is then annealed to immobilised oligonucleotides in the array, as shown in (B). The expressed peptide can then be detected by any suitable mechanism, such as the specific binding of a protein (e.g. a fluorescently labelled antibody or an antibody conjugated to an enzyme that can be used with a fluorescent reagent), as shown in (C). The DNA template is prepared for sequencing as shown in panels (D) to (I). The appropriate regions of the single-stranded molecules are sequenced and the templates are then denatured, followed by a fill-in reaction to remake the full double-stranded molecule. In a variation of this protocol, in step (B) the peptide-nucleic acid complexes may be annealed to oligonucleotides in solution and then immobilised onto the array.

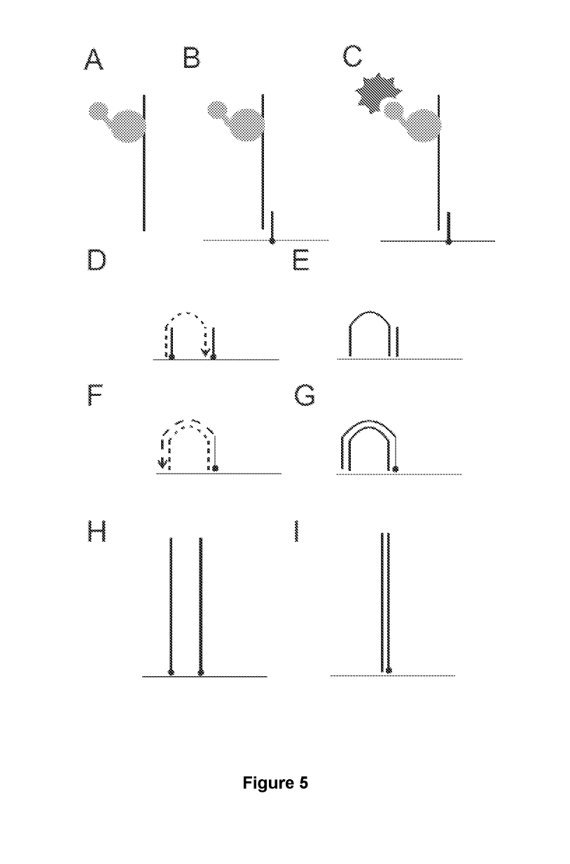

FIG. 6 shows the process of sequencing a DNA template on a bead (A); followed by fill-in using a polymerase (B); and transcription and translation (C), so that protein is expressed and binds back to its own encoding DNA through the binding of an appropriate coupling mechanism (e.g. RepA to ori). The expressed peptide can then be detected by the specific binding of a protein, such as a fluorescently labelled antibody or an antibody conjugated to an enzyme that can be used with a fluorescent reagent (D).

FIG. 7 demonstrates a sequencing and selection procedure in accordance with an alternative aspect in the invention for identifying peptide-binding pairs. Members of a first nucleic acid library (Library 1, light grey) containing different members are immobilised on a surface, and proteins containing each member of the peptide library are then expressed by an in vitro transcription/translation reaction and bind back to their own respective DNA template molecule (e.g. via an ‘anchoring’ sequence), as described elsewhere. A second library (Library 2, dark grey)—not immobilised—is similarly made using an in vitro transcription/translation procedure and the members of this library are also bound to their respective DNA templates. In a subsequent selection procedure, following sequence analysis of Library 1 and creation of the protein-DNA fusions displaying immobilised peptide library members, the Library 2 peptide-DNA fusions are passed over the flow cell containing immobilised Library 1 peptide-DNA fusions, and members of Library 2 that bind to peptide members of Library 1 can be identified by a fluorescent tag attached to the DNA (or the Library 2 protein). The bound complexes of Library 1 and Library 2 peptides can then be removed from the surface by specific cleavage (for example, irradiation at 320 nm with a laser focused upon the cluster of interest). Specific binding clusters can be cherry picked from the array using this approach, as illustrated by the diagonal arrow in panel (A). A laser or lasers can be directed to the appropriate spots for specific release of the complexes of Library 1 and Library 2 (B and C). The beam of the laser may be moved to release different complexes in a desired order, as illustrated in panels A, B and C.

FIG. 8 shows an alternative embodiment to that of FIG. 7, in which Library 1 binds to a labelled nucleic acid library (Library 2) that has not be subjected to transcription/translation.

FIG. 9 shows an alternative embodiment to that of FIG. 7, in which the sequencing and selection beads are trapped in the picoliter wells of a Roche or Ion torrent sequencing chip. In this embodiment, nucleic acid members of Library 1 are sequenced and then subjected to transcription and translation to form immobilised peptide-DNA complexes. These complexes are then exposed to peptide-nucleic acid complexes from Library 2 (not immobilised), and binding members are identified through fluorescent tags on Library 2 DNA or proteins. The Library 1 and Library 2 complexes can then be released specifically from the beads, e.g. by irradiation at 320 nm using a suitable laser (B). Alternatively, individual beads might be released by other means such as a magnet, pressure difference or electrical stimulation.

### DETAILED DESCRIPTION OF THE INVENTION

In order to assist with the understanding of the invention several terms are defined herein.

The term ‘peptide’ as used herein refers to a plurality of amino acids joined together in a linear or circular chain. The term ‘oligopeptide’ is typically used to describe peptides having between 2 and about 50 or more amino acids. Peptides larger than about 50 are often referred to as ‘polypeptides’ or ‘proteins’. For purposes of the present invention, the term peptide is not limited to any particular number of amino acids. Preferably, however, they contain up to about 400 amino acids, up to about 300 amino acids, up to about 250 amino acids, up to about 150 amino acids, up to about 70 amino acids, up to about 50 amino acids or up to about 40 amino acids. Suitably, a modified peptide of the invention contains between about 10 and about 60 amino acid residues and more suitably between about 15 and about 50 residues, between about 18 and about 45 residues, or between about 20 and about 40 residues. In some embodiments a peptide of the invention may contain about 22 to about 38 amino acid residues, or between about 24 and about 36 residues: for example, 25, 26, 27, 28, 29, 30, 31, 32, 33, 34 or 35 amino acids. It should be understood that an isolated or modified peptide of the invention may comprise or consist of the above number of amino acids. In some aspects and embodiments, the ‘peptide’ is an antibody or an antibody fragment comprising at least one polypeptide chain that is not a full-length antibody chain, such as: (i) a Fab fragment, which is a monovalent fragment consisting of the variable light (V_{L}), variable heavy (V_{H}), constant light (C_{L}) and constant heavy 1 (C_{H}1) domains; (ii) a F(ab′)2 fragment, which is a bivalent fragment comprising two Fab fragments linked by a disulphide bridge at the hinge region; (iii) a heavy chain portion of an Fab (Fd) fragment, which consists of the V_{H} and C_{H1} domains; (iv) a variable fragment (Fv) fragment, which consists of the V_{L} and V_{H} domains of a single arm of an antibody, (v) a domain antibody (dAb) fragment, which comprises a single variable domain; (vi) an isolated complementarity determining region (CDR); (vii) a Single Chain Fv Fragment; (viii) a diabody, which is a bivalent, bispecitc antibody in which V_{H} and V_{L} domains are expressed on a single polypeptide chain, an engineered constant domain such as Ckappa or Clambda, C_{H}1, C_{H}2, C_{H}3 or C_{H}4.

The term ‘amino acid’ in the context of the present invention is used in its broadest sense and includes naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further encompasses D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The expressed peptides of the invention (i.e. those subjected to a screening/selection procedure) may be designed de novo, may be completely random peptide sequences, or may be derived from a protein, or a fragment or domain of a protein, e.g. which has been diversified by randomisation of one or more amino acid position. Randomisations for diversification of peptide sequences may be full, partial and/or selective, so as to include completely random libraries as well as libraries in which selected positions are partially diversified using defined groups of amino acids.

Peptide libraries used in accordance with the invention are created using a diversified nucleic acid population in which the codon for an amino acid position to be diversified is varied using appropriate nucleic acids at appropriate positions of the codon, according to the desired library diversity at that position, as known by the skilled person in the art. For example, all natural amino acids can be encoded by the codons NNN and NNB, whereas less diversified codons can be used to encode a sub-group of amino acids. Nucleic acid triplets (e.g. MAX codons) can also be used for DNA synthesis to ensure that a particular codon of the nucleic acid library encodes a desired group of amino acids, as described, for example, in Hughes et al. (2005) Nucleic Acids Res. 33:e32. The invention is particularly beneficial for the selection of peptides having desired properties from naïve peptide/nucleic acid libraries. By ‘naïve’ it is meant that the library members (peptides) have not previously been exposed to the target molecule and the library is not, therefore, pre-enriched for potential binding members. A particular benefit of the invention is that selection from a naïve library (e.g. containing at least 10⁶, at least 10⁸, at least 10¹⁰ members or more as described herein) can be achieved in a single round/screen without pre-enrichment of the library. Furthermore, after this single round the peptides of interest are already characterised at least by virtue of the nucleic acid sequence that encodes it.

Once a peptide library member having a desired phenotype/characteristic has been selected it may be further modified or matured. A ‘modified’ peptide of the invention may have been mutated (e.g. by an amino acid substitution, deletion, addition) in at least one position. It will be appreciated that a peptide or modified peptide of the invention may comprise an additional peptide sequence or sequences at the N- and/or C-terminus, e.g. for improving peptide expression or nucleic acid cloning: for example, the dipeptide sequence met-ala may be included at the N-terminus.

Modified peptides of the invention typically contain naturally occurring amino acid residues, but in some cases non-naturally occurring amino acid residues may also be present. Therefore, so-called ‘peptide mimetics’ and ‘peptide analogues’, which may include non-amino acid chemical structures that mimic the structure of a particular amino acid or peptide, may also be used within the context of the invention. Such mimetics or analogues are characterised generally as exhibiting similar physical characteristics such as size, charge or hydrophobicity, and the appropriate spatial orientation that is found in their natural peptide counterparts. A specific example of a peptide mimetic compound is a compound in which the amide bond between one or more of the amino acids is replaced by, for example, a carbon-carbon bond or other non-amide bond, as is well known in the art (see, for example Sawyer, in Peptide Based Drug Design, pp. 378-422, ACS, Washington D.C. 1995). Such modifications may be particularly advantageous for increasing the stability of a peptide and/or for improving or modifying solubility, bioavailability and delivery characteristics (e.g. for in vivo applications).

Modified peptides of the invention also encompass ‘derivatives’ of peptides selected in accordance with the invention. A ‘derivative’ of a peptide identified by a method of the invention has the selected desired activity (e.g. binding affinity for a selected target ligand), but, like a modified peptide of the invention, may further include one or more mutations or modifications to the primary amino acid sequence of the peptide. For example, it may have one or more (e.g. 1, 2, 3, 4, 5 or more) chemically modified amino acid side chains. Suitable modifications may include pegylation, sialylation and glycosylation. These may be incorporated through non-natural amino acids or through chemical modification of the natural sequence. In addition (as noted above) or alternatively, a derivative may contain one or more (e.g. 1, 2, 3, 4, 5 or more) amino acid mutations, substitutions or deletions to the primary sequence of the peptide from which it is derived. Accordingly, the invention encompasses the results of maturation experiments conducted on a selected peptide to improve or alter one or more of its characteristics. By way of example, to mature a peptide towards a desirable characteristic one or more amino acid residue of the peptide sequence may be randomly or specifically mutated (or substituted) using procedures known in the art (e.g. by modifying the encoding DNA or RNA sequence). The resultant library or population of derivatised peptides may then be further selected, by any known method in the art, according to predetermined requirements: such as improved specificity against a particular target ligand; or improved drug properties (e.g. stability, solubility, bioavailability, immunogenicity etc.). Peptides selected to exhibit such additional or improved characteristics and that display the activity for which the peptide was initially selected may be considered to be derivatives of the peptides of the invention and fall within the scope of the invention.

Where the selected phenotype relates to binding of a nucleic acid or peptide library member to a target molecule or ligand, the screening/selection process is advantageously not restricted to a particular type or conformation of molecule or ligand (e.g. such as a linear peptide). Thus, any desirable ligand may be recognised (i.e. bound) by library members, including nucleic acids (e.g. DNA or RNA), small organic or inorganic molecules, carbohydrates, proteins or peptides. In some embodiments, a suitable ligand may be a protein, and a particularly suitable ligand is a peptide sequence, such as a (surface) ‘epitope’ or an active site or cleft peptide sequence/surface of a protein target. Preferred target ligands may be linear peptides, which may be isolated or part of a larger peptide or protein molecule.

The library may comprise a plurality of nucleic acid sequences (e.g. at least 10⁶, 10⁸, 10¹⁰, 10¹² or more different coding sequences) that may be expressed and are screened to identify nucleic acids or peptides having a desired property. Preferred systems for expression and screening of libraries are ‘in vitro peptide display’ systems, which are capable of generating large libraries sizes, and of being performed in in vitro systems, such as on solid substrates and/or in sequencing-compatible platforms. The terms ‘in vitro display’, ‘in vitro peptide display’ and ‘in vitro generated libraries’ as used herein refer to systems in which peptide libraries are expressed in such a way that the expressed peptides associate with the specific nucleic acids that encoded them, and the association does not follow or require the transformation of cells or bacteria with the nucleic acids. Accordingly, these systems can be considered to be ‘acellular’ or ‘cell free’. Such systems contrast with phage display and other ‘cellular’ or ‘in vivo display’ systems in which the association of peptides with their encoded nucleic acids follows the transformation of cells or bacteria with the nucleic acids. In a preferred embodiment of the invention, the CIS-display system (for example, as described in WO2004/022746, WO2006/097748 and WO2007/010293) is used as an in vitro display system.

In particular, cell-free systems may be selected from E. coli or other prokaryotic or eukaryotic systems, such as from wheat germ or rabbit reticulocytes, or alternatively from an artificially reconstructed system, such as the Puresystem. In yet other alternatives, the cell-free system may comprise a mixture of different systems, or systems that have been modified through the addition of reagents to assist with protein folding, such as chaperones (protein chaperones or artificial chaperones such as polysaccharide compounds), or compounds that modulate the formation of disulphide bonds, such as oxidised and reduced glutathione, which systems enable the synthesis of polypeptides.

Another useful peptide-library generation system that may be employed to link genotype and phenotype in the methods of the present invention is ‘ribosome display’, as described for example in “Ribosome Display and Related Technologies”, edited by Douthwaite & Jackson, 2012, Methods in Molecular Biology, Volume 805, Springer Press, Mattheakis et al., (1994) PNAS, 91, 9022-9026; Hanes and Pluckthun (1997) PNAS, 94, 4937-4942; He and Taussig (1997) NAR, 25, 5132-5134; Nemoto et al., (1997) FEBS Lett. 414, 405-408; Robers and Szostak, (1997) PNAS, 94, 12297-12302; Tawfik & Griffiths, (1998) Nat. Biotech., 16, 652-656; Odegrip et al., (2004) PNAS, 101, 2806-2810; Reiersen et al., (2005) NAR, 33 e10; Bertschinger et al., (2007) Protein. Eng. Des. Sel., 20, 57-68; and in patent applications WO1998/031700; WO1998/016636; WO1998/048008; WO1995/011922; W02011/0183863; and WO2004/022746 and as reviewed by Ullman et al., (2011) Brief Funct Genomics; 10, 125-134). An approach to link peptides on plasmids inside bacterial cells might also provide a suitable system and substrate for the performance of peptide binding studies—see e.g. Cull et al., (1992) Proc Natl. Acad. Sci. USA, 89:1865-9. The use of cross-linkers to stabilise peptide-DNA interactions might also be beneficial. Suitable cross-linking chemistries include primary amines covalently linked to an activated carboxylate group or succinimidyl ester, thiols covalently linked via an alkylating reagent such as maleimide.

### Immobilisation of Nucleic Acids and Arrays

The library of nucleic acid molecules for in situ sequencing and screening is suitably immobilised. Nucleic acids may be immobilised using any suitable system known to the person of skill in the art, and which is compatible with the chosen sequencing and screening protocols. For example, the immobilising may be a covalent or non-covalent attachment to a solid support. The term ‘immobilisation’ is used in its broadest sense to encompass all appropriate forms of capturing or attaching the nucleic acid to the support. The term ‘attachment’ is used herein interchangeably with terms such as ‘linked’, ‘bound’, ‘conjugated’ and ‘associated’, and such terms may also be used to describe suitable forms of immobilisation.

A wide range of covalent and non-covalent forms of conjugation are known to the person of skill in the art, and fall within the scope of the invention. For example, disulphide bonds, chemical linkages and peptide chains may all provide suitable forms of covalent linkages. Where a non-covalent means of conjugation is preferred, the means of attachment may be, for example, a biotin-(strept)avidin link or the like. Typically, one or more nucleic acid strands of the molecule to be immobilised is modified with a group that can be linked to a compatible moiety on a solid support. Suitable immobilisation chemistries include amine-modified nucleic acid molecules covalently linked to an activated carboxylate group or succinimidyl ester, thiol-modified nucleic acid molecules covalently linked via an alkylating reagent such as an iodoacetamide or maleimide; acrydite-modified nucleic acid molecules covalently linked through a thioether; and biotin-modified nucleic acid molecules captured by immobilised streptavidin. Surface immobilisation chemistries are well known in the art and include, for example, antibody (or antibody fragment)-antigen interactions that may also be suitably employed to immobilise a nucleic acid molecule. One suitable antibody-antigen pairing is the fluorescein-antifluorescein interaction.

Suitable substrates or solid supports for arrays should be non-reactive with reagents to be used in processing, washable (e.g. under stringent conditions), not interfere with nucleic acid hybridisation and sequencing, and not be subject to non-specific binding reactions etc., which might interfere with peptide selection procedures. They must also, of course, be amenable to covalent or non-covalent linking of oligonucleotides for immobilisation. Suitable support materials are well known in the art, and include, for example, treated glass, polymers of various kinds (e.g. polyamide, polystyrene and polyacrylmorpholide), polysaccharides (e.g. Sepharose, Sephadex and dextran), latex-coated substrates, silica chips and metal surfaces. Preferred solid supports are beads (e.g. latex beads) that may beneficially be paramagnetic in property, microtitre plates (e.g. in 96- or 384-well format), or micro/silica chips.

The type of solid support to be used will typically determine the way in which the array is manufactured. The appropriate methods for immobilisation of nucleic acids on different solid supports are well known in the art. For example, where the support is made of glass the surface may be coated with long aminoalkyl chains (e.g. Ghosh & Musso (1987), Nucleic Acids Res. 15, pp 5353-5372); other immobilisation surfaces include a polyacrylamide layer (e.g. Khrapko et al., (1989), FEBS Lett., 256, pp 118-1223); latex (Kremsky et al., (1987), Nucleic Acids Res., 15, pp 2891-29093); or various polymers (Markham et al., (1980), Nucleic Acids Res., 8, pp 5193-5205; Norris et al., (1980), Nucleic Acids Symp. Ser., 7, pp 233-241; Zhang et al., (1991), Nucleic Acids Res., 19, pp 3929-3933).

Double-stranded nucleic acid molecules can be directly immobilised onto the support, or alternatively a single-stranded oligonucleotide may be immobilised on the support followed by synthesis of the second strand to create a double-stranded molecule. Various methods of oligodeoxyribonucleotide synthesis directly on a solid support are known in the art. In some cases, synthesis may occurs in the 3′ to 5′ direction so that the oligonucleotides can possess free 5′ termini (e.g. Caruthers et al., (1987), Methods Enzymol., 154, pp 287-313; Horvath et al., (1987), Methods Enzymol., 154, pp 314-326); and other methods synthesise nucleotides in the 5′ to 3′ direction so that the oligonucleotides may possess free 3′ termini (e.g. Agalwal et al., (1972), Angew. Chem., 11, pp 451-459; Belagaje & Brush (1982), Nucleic Acids Res., 10, pp 6295-6303; Rosenthal et al., (1983), Tetrahedron Lett., 24, pp 1691-1694; Barone et al., (1984), Nucleic Acids Res., 12, pp 4051-4061).

Similarly, there are also various methods known in the art for the synthesis of oligoribonucleotides or mixed DNA/RNA oligonucleotides directly on a solid support (e.g. Scaringe et al., (1990), Nucleic Acids Res., 18, pp 5433-54413; Veniaminova et al., (1990), Bioorg. Khim. (Moscow), 16, pp 941-950; and Romanova et al., (1990), Bioorg. Khim. (Moscow), 16, pp 1348-1354).

Methods for the simultaneous synthesis of many different oligonucleotides is also known in the art (Frank et al., (1987), Methods Enzymol., 154, pp 221-249; Djurhuus et al., (1987), Methods Enzymol., 154, pp 250-287).

Depending on the type of array and the desired procedure, oligonucleotides may be synthesised on an array by washing over the array one or more nucleotide (G, A, T/U and C) for incorporation into the growing strand. In this way, each immobilised nucleotide in the array may be exposed simultaneously to the one or more nucleotides. Alternatively, one or more nucleotide may be delivered directly and specifically to one or more immobilised nucleotide. Arrays are particularly suitable for the automated delivery of different nucleotide precursors to precise locations, for example, using a computer-controlled device, such as a modified inkjet printer (drop-on-demand′ technology), or photolithography technique (Fodor et al., (1991), Science, 251, pp 767-773). Such techniques are also suitable for the production of the array and the delivery of oligonucleotides to defined positions on an array for immobilisation.

Depending on the technology employed and the library design/size, arrays can be made over a range of sizes (e.g. in the millimetre range) and densities (e.g. 256×256; 512×512 etc.), or these can be in the μm or sub μm range as described for the CMOS node (see e.g. Rothberg et al. (2011), Nature, 475, 348-352). Arrays can be made in any shape or arrangement, which may be determined by the robotic equipment used to construct the array, and the manner in which it is to be screened. Typically, an array is ordered (although random arrays are also suitable), and may be in the form of a square, rectangle, line, (concentric) circles, or spiral.

### Nucleic Acid (Next-Generation) Sequencing

In accordance with the invention, any form of sequencing procedure suitable for use on immobilised (e.g. arrayed) oligonucleotide templates may be used. Most suitable sequencing techniques are, therefore, the second- or next-generation sequencing techniques, since these are particularly adapted for use with immobilised or arrayed templates. Exemplary next-generation sequencing procedures are outlined below and these are particularly preferred for use in the present invention.

Since sequencing techniques generally involve filling in/extension of the second complementary strand of a single-stranded template, it can be convenient to sequence the oligonucleotide library members before synthesis of a double-stranded oligonucleotide for use in transcription and translation. Thus, in one embodiment the immobilised oligonucleotides are sequenced in situ prior to expression and screening of their corresponding peptides. For this purpose, therefore, in some embodiments it is beneficial to immobilise single-stranded or only partially double-stranded oligonucleotides for sequencing. After sequencing, a double-stranded oligonucleotide may be present that can be used directly for transcription and/or translation. However, it may be efficient to only sequence a portion of the oligonucleotides in the library (e.g. the region of randomisation or diversification). This is particularly beneficial for use in conjunction with some next-generation sequencing procedures, which may have relatively short read lengths of e.g. less than 200 bases. In such embodiments, before expression of the peptide library, double-stranded oligonucleotide synthesis may be completed or carried out de novo by a suitable technique, such as by primer extension. Alternatively, the short double-stranded template encoding at least the peptide library portion of the protein to be expressed may be joined (e.g. by restriction digestion and ligation) to a double-stranded portion encoding a constant portion of the protein to be expressed as a fusion with the peptide library portion. For example, it is particularly convenient for the portion of the nucleic acid encoding a cis-binding protein, antibody (fragment), tag sequence or similar, which is constant in all members of the nucleic acid and peptide library to be appended to the library portion after sequencing.

### Pyrosequencing

The 454 pyrosequencing method differs from Sanger sequencing, in that it relies on the detection of pyrophosphate release on nucleotide incorporation, rather than chain termination with dideoxynucleotides. A single-stranded DNA strand is sequenced by synthesising its complementary strand enzymatically, one base pair at a time, and detecting which base was actually added at each step. The method is broadly based on the detection of DNA polymerase activity with another chemiluminescent enzyme, and light is produced only when a nucleotide is correctly added to the growing strand. These chemiluminescent signals are used to elucidate the template sequence.

First, template DNA molecules are immobilised and a sequencing primer than hybridises to an appropriate point 5′ of the region to be sequenced is annealed to the template. The immobilised oligonucleotides are then incubated with the enzymes DNA polymerase, ATP sulfurylase, luciferase and apyrase, and with the substrates adenosine 5′ phosphosulfate (APS) and luciferin. Solutions of A (generally dATPαS, which is not a substrate for a luciferase, is added instead of dATP), C, G, and T nucleotides are sequentially added and removed from the reaction to extend the sequencing primer. DNA polymerase incorporates the correct, complementary dNTPs onto the template and causes the release of stoichiometric amounts of pyrophosphate (PPi). The released PPi is then converted into ATP by ATP sulfurylase in the presence of adenosine 5′ phosphosulfate. The produced ATP then enables luciferase-mediated conversion of luciferin to oxyluciferin, in a process that generates visible light in amounts that are proportional to the amount of ATP. The light produced in the luciferase-catalysed reaction can be detected by a camera and analysed by appropriate computer software to determine the location of the signal. After the addition of each nucleotide unincorporated nucleotides and ATP are degraded by apyrase, so that the reaction can be restarted with another nucleotide.

The templates for pyrosequencing can be made both by solid phase template preparation (e.g. streptavidin-coated magnetic beads) or enzymatic template preparation (apyrase and exonuclease).

One suitable pyrosequencing procedure is the 454 pyrosequencing technique (454 Life Sciences, Roche Diagnostics).

In some embodiments, the pyrosequencing technique makes use of emulsion-PCR.

By way of example, a polyclonal mixture of DNA fragments may be separated and clonally amplified through the capture of a DNA molecule onto the surface of a 28 μm bead, which is then trapped within a droplet of a water-in-oil emulsion and amplified through PCR. This can result in each bead carrying in the region of 10,000,000 copies of the same DNA template. The beads can then be released from the emulsions, washed, treated with Bacillus stearothermophilus (Bst) polymerase and a single-stranded binding protein and passed over an array of picoliter sized wells. These are large enough (44 μm diameter by 50 μm deep) to capture a single bead (and hence a single library sequence) in each well.

The sequencing reactions flow over the surface of the array in a 300 μm high channel and the base of the array is connected to a charge-coupled device which captures the emitted photons from the bottom of each well. Primers and smaller beads carrying immobilised enzymes are added to the wells to perform the sequencing process generally as described above. Cyclically delivered reagents flow perpendicularly into the wells, and where an unlabelled nucleotide is incorporated into the DNA, pyrophosphate is released which is acted upon by ATP sulfurylase and luciferase, using adenosine 5′-phosphosulphate and luciferin as substrates, to generate a photon of light that is detected by the CCD and correlated to the location of the well. An apyrase enzyme wash then removes unincorporated bases. Thus with iterative cycles of base addition, the sequence of the DNA immobilised on the surface of the beads can be recorded (see e.g. Margulies et al., (2005), Nature, 435, pp 376-380; and Shendure and Ji (2008), Nature Biotechnol., 26, pp 1135-1145; Rothberg and Leamon (2008) Nature Biotechnol., 26, pp 1117-1124; Mardis (2008), Annu. Rev. Genomics. Hum. Genet., 9, 387-402; and Gupta (2008) Trends Biotechnol., 26, 602-611).

### SOLiD™ Sequencing

For use in the Applied Biosystems (AB) SOLiD™ system a library of DNA fragments is prepared and used to create clonal bead populations (e.g. by emulsion-PCR) such that only one species of oligonucleotide is present on the surface of each magnetic bead. Beneficially, a universal adapter sequence (e.g. universal P1 adapter sequence) is attached to each of the immobilised nucleic acids to be sequenced so that the starting sequence of every fragment is known and identical. The beads are then immobilised on a planar substrate (e.g. a glass slide) to form an array (Shendure & Ji (2008), Nature Biotechnol., 26, 1135-1145; Mardis (2008), Annu. Rev. Genomics. Hum. Genet., 9, 387-402).

To begin the sequencing reaction, primers are hybridised to the P1 adapter sequence within the library template. The sequencing reaction is driven by ligation of oligonucleotides that hybridise to the single-stranded region adjacent to the adapter using DNA ligase. In one embodiment, the oligonucleotides are octamers that are fluorescently labelled in their fourth and fifth positions, which provides a readout for these positions of the template. The hybridised oligonucleotide is then cleaved and the process repeated. Multiple cycles of ligation, detection and cleavage are performed, with the number of cycles determining the eventual read (sequencing) length, thus generating sequences for the 4^{th}, 5^{th}, 9^{th}, 10^{th}, 13^{th} and 14^{th} positions and so on. Once the entire sequence has been read in this fashion, the process is repeated with shorter oligonucleotides to read first the 3^{rd}, 4^{th}, 8^{th}, 9^{th}, 13^{th} and 14^{th} positions; and sequentially then positions 2, 3, 7, 8, 12 and 13; and finally positions 1, 2, 6, 7, 11 and 12, to generate a complete sequence. Through this process, each base position is interrogated in two independent ligation reactions by two different primers.

In an alternative embodiment of the emulsion PCR process, the emulsions may be ruptured and the beads are separated into picowells on the surface of an electrochemical sensor (as described in relation to pyrosequencing). On incorporation of a base, a hydrogen ion is released that then creates a minute change in pH that can be detected by an electrochemical detector, such as an ion-sensitive field effect transistor (ISFET) (e.g. as used in the Ion Torrent sequencing method).

### Ion Torrent Sequencing

Ion Torrent sequencing (also known as ion semiconductor sequencing) is a method for DNA sequencing that is based on the detection of hydrogen ions that are released during the polymerisation of DNA. This technology differs from other sequencing technologies in that no modified nucleotides or optics are used and nucleotide incorporation is detected by the release of pyrophosphate and a positively charged hydrogen ion following the formation of a covalent bond between adjacent deoxyribonucleotides. This causes a small change in the pH of the environment which is only produced when a nucleotide extension occurs. The signal also is proportional to the number of hydrogen ions released so that homopolymer stretches can be correctly interpreted. The electrical signal that is generated can be converted to a DNA sequence. Signal processing and DNA assembly can then be carried out using the appropriate software (see e.g. Rothberg et al., 2011, Nature 475, 348-352; US2010/0282617; US2011/0287945).

### Illumina/Solexa Sequencing

Illumina (Solexa) technology operates on a planar surface using ‘bridge-PCR’ to generate thousands of clonal copies of a DNA fragment (or oligonucleotide) for sequencing (see e.g. Mardis (2008), Annu. Rev. Genomics Hum. Genet. 9, pp 387-402; Bentley et al., (2008), Nature, 456, 53-59; and U.S. Pat. No. 7,232,656).

In brief, DNA oligonucleotides are ‘end-labelled’ with appropriate adapter sequences suitable for hybridisation to primers for PCR. The oligonucleotides are then denatured (if double-stranded) to generate a single-stranded molecule with known end sequences, and hybridised to a support/surface onto which a large number of forward and reverse primer adapters have already been attached via a flexible linker. The single-stranded oligonucleotide is immobilised at one end and its free end is thus able to flex in order to find and pair with the immobilised primer that is complementary to that end. Multiple cycles of PCR amplification (bridge PCR′) are carried out to generate e.g. approximately 1,000 copies of each template clustered in close proximity to each other on the surface. Millions of such clonal clusters (each potentially having a different sequence) can be accommodated in a single array. After each cycle in DNA amplification (e.g. using Bst polymerase), formamide denaturation of the double-stranded products may be used to generate single stranded templates for the next round of amplification.

For sequencing, a different primer may be used to amplify the region of interest, and a modified polymerase and four differently labelled fluorescent terminator bases can be added to e.g. the flow cell, so that the bases that are incorporated can be specifically detected. After each cycle of sequencing, the fluorescent moiety and the 3′ hydroxyl block are then chemically removed so that the cycle can be repeated through addition of the next labelled nucleotide.

### HeliScope™ Sequencing

The HeliScope™ approach does not require clonal amplification and is able to determine the sequence of single DNA molecules using a highly sensitive fluorescence detection system known generally as single-molecule fluorescent sequencing.

First, DNA oligonucleotides are prepared and immobilised on a planar surface. Typically, this is carried out by poly-A tailing of the oligonucleotide so that it can be immobilised onto the surface (e.g. of a flow cell) using previously immobilised poly-T oligonucleotide anchors, to yield a randomly distributed array of hybridised DNA templates for sequencing. The polymerase and a single species of fluorescently labelled nucleotide are then added, and single base incorporation can be detected by exciting the fluorophore with a laser and detecting the release of photons. After any incorporated nucleotides have been detected the fluorescent label can be cleaved from the oligonucleotide and removed by washing, so that a new polymerase and different fluorescently-labelled nucleotide can be added. Conveniently, the fluorophore may be conjugated to the nucleotide via a disulfide bridge which can be readily cleaved to remove the fluorescent group. This procedure is then repeated until all four fluorescently-labelled bases have been added in turn; and multiple cycles of the procedure thus allow the sequencing of the template (see for example, http://helicosbio.com/Portals/O/Documents/Helicos%20tSMS%20Technology%20Primer. pdf; Gupta, (2008), Trends Biotechnol., 26, 602-611).

### Proteins, Peptide Libraries and Expression

The present invention is suitable for the expression and screening/selection of any protein or peptide sequence for any desirable properties, such as binding affinity to a chosen target ligand.

Suitably, the protein, protein fragment or domain, or peptide to be screened for a particular activity contains up to about 100 amino acids, such as up to 50 amino acids. However, longer or shorter members of a peptide library may of course be expressed. In addition, the protein, protein fragment or domain, or peptide to be screened is advantageously conjugated (e.g. fused) to a cis-binding agent (e.g. a protein or protein fragment or domain) or other protein tag/binding agent, which is suitable for cis-binding to its encoding nucleic acid sequence. The encoding nucleic acid sequence being comprised in an immobilised oligonucleotide, which in some embodiments includes a nucleic acid (anchoring′) sequence that can be recognised and bound by the cis-binding protein. In this way, the expressed protein or peptide to be screened is linked (immobilised) via the cis-binding agent to its encoding nucleic acid molecule, so that the peptide to be screened is immobilised in the same location as its encoding DNA.

Convenient cis-binding agents include cis-acting proteins (CAPs; see e.g. Lindqvist, WO98/37186; and Odegrip, WO2004/022746). Two suitable such proteins are the A protein from P2 phage (P2A), and the RepA replication initiator protein from the R1/R100 plasmid. A preferred cis-element is a binding site for a nucleic acid-binding domain and, thus, may conveniently be formed by a sequence within the library oligonucleotide. It may be located 5′ or 3′ of the gene-encoding sequence. However, other alternative cis-binding agents may be used, as known in the art, such as (strept)avidin, which can bind to a biotin moiety (e.g. attached to the encoding nucleic acid); or suitable antibodies or antibody fragments or domains, which may recognise epitopes or small molecules conjugated (e.g. by chemical linkers) to the nucleic acid molecule.

Advantageously, where the expressed peptides comprise cis-binding proteins, fragments or domains, the nucleic acid library sequence may further comprise a stalling sequence, which stalls (or pauses) an RNA polymerase transcribing the DNA sequence. In this way, the transcription complex comprising DNA, RNA polymerase, RNA, ribosome and nascent peptide is (temporarily) locked. Thus, the nascent peptide has enough time to correctly fold, and recognise and bind to its nearest binding sequence, such as an on (origin of replication) sequence, which is generally on its encoding DNA molecule. One preferred stalling sequence is a cis-element that contains a transcription termination sequence (C/S sequence), although alternative sequences may be used.

A preferred in vitro protein expression and screening system for use in the present invention is a CIS in vitro display system, such as described in Odegrip et al., (2004, PNAS, 101, 2806-2810) and e.g. WO2004/022746, which are incorporated herein by reference.

Alternative systems that operate acellularly are based upon stalling of the ribosome on the mRNA template (ribosome or polysome display′) so that the nascent peptide remains in a complex, which could then be disrupted by EDTA, for example. The released RNA can be subsequently amplified by an RT-PCR step. Both bacterial and eukaryotic systems have been developed (Hanes 1998, 1999; He & Taussig 2002 supra). The absence of a stop codon to stall the ribosomes and a C-terminal peptide spacer to try to ensure that the folding of the displayed polypeptide is not sterically hindered by the ribosomal tunnel are generally important features of this technology.

A related technique, mRNA (or in vitro virus) display differentiates itself from ribosome display by the formation of a covalent link between the template and the expressed protein, e.g. via puromycin. Puromycin is carried on a DNA primer appended to the mRNA template and mimics amino-acyl tRNA, thus binding covalently to the nascent peptide as a result of the peptidyl transferase activity of the ribosome. The DNA primer is then used in a reverse transcription step to stabilise the RNA template in a RNA/DNA hybrid (e.g. as reviewed by Takahashi 2003, Trends in Biochemical Sciences, 28, 159-165; Millward et al., 2007, ACS Chemical Biology, 2, 625-634; and Wilson et al. 2001, PNAS, 98, 3750-3755). A variant of mRNA display which replaces the RNA with a double stranded DNA molecule using modified linkers has also been described and may find utility in an alternative embodiment of the invention (see review by Douthwaite & Jackson, “Ribosome Display and Related Technologies”, edited by Douthwaite & Jackson, 2012, Methods in Molecular Biology, Volume 805, Springer Press; and Ullman et al., (2011), Briefings in Functional Genomics, 10, 125-134; and as described in W02011/0183863).

The amino acid residues at each of the mutated positions in the library may be non-selectively randomised, e.g. by incorporating any of the 20 naturally occurring amino acids. When the library is based on a known protein, a non-selective randomisation implies replacing each of the specified amino acids with any one of the other 19 naturally occurring amino acids. Alternatively, the diversified positions may be selectively randomised, by incorporating any one from a defined sub-group of amino acids at the appropriate position. The mutations and diversifications may also encompass non-natural amino acids.

It will be appreciated that one convenient way of creating a library of mutant peptides with randomised amino acids at each selected location, is to randomise the nucleic acid codon of the corresponding nucleic acid sequence that encodes the selected amino acid. In this case, in any individual peptide expressed from the library, any of the 20 naturally occurring amino acids may be incorporated at the randomised position. Therefore, when the library is derived from a wild-type protein sequence, in some instances (e.g. approximately 5%), the wild-type amino acid residue may be ‘randomly’ incorporated by chance. By contrast, by substituting a selected amino acid of a wild-type sequence with one from a defined sub-group of amino acids (e.g. by intelligent/selective codon randomisation), it can be pre-determined whether or not any of the library members might incorporate a wild-type residue at the selected location by chance. Likewise, it can be determined which amino acids have the chance of being incorporated in a particular position. Beneficially, randomisation codons can be selected that avoid incorporation of STOP codons (so as to avoid producing truncated peptides), or to avoid certain undesirable amino acids at a particular position, as is known in the art. A most suitable method of generating a peptide sequence with a desired randomisation pattern is by synthesising the encoding nucleic acid using trinucleotide building blocks, e.g. using MAX codon synthesis methods.

Alternatively precharged tRNAs may be used to introduce non-natural amino acids at any one or more of the amino acid positions to be mutated. Other methods of tRNA aminoacylation with non-natural amino acids include the use of ribozymes or mutated aminoacyl-tRNA synthetases (AARS) which may have specific four base codons (Ullman et al., (2011), Briefings in Functional Genomics, 10, pp 125-134).

Where the expression and screening system involves a CAP, the library peptide may be beneficially expressed as a fusion protein with the CAP, domain or fragment. This provides for convenient expression, screening and selection of desirable peptides. In one embodiment, library peptides include a suitable amino acid linker (e.g. GSGSS; SEQ ID NO: 61) at the C-terminus or N-terminus for fusion to the CAP sequence, and the encoding nucleic acid library sequence thus includes a corresponding nucleic acid linker sequence. Such a linker is convenient for fusing library peptides for use in accordance with the invention to the RepA protein for expression and selection in a CIS in vitro display system. In another embodiment the library may be encoded within a loop of the CAP.

### Characterisation of Peptides

Where it is desired to identify peptides from a library that have binding affinity (or improved binding affinity) for a defined target epitope or molecule, the peptide(s) selected can be subsequently characterised by measuring binding affinity of the isolated peptide to the target molecule.

The binding affinity of a selected peptide for the target ligand can be measured using techniques known to the person of skill in the art, such as tryptophan fluorescence emission spectroscopy, isothermal calorimetry, surface plasmon resonance, or biolayer interferometry. Biosensor approaches are reviewed by Rich et al. (2009), “A global benchmark study using affinity-based biosensors”, Anal. Biochem., 386, 194-216. Alternatively, real-time binding assays between the peptide and ligand may be performed using biolayer interferometry with an Octet Red system (Fortebio, Menlo Park, Calif.).

Alternatively, the desired property of the peptide may be an activity, such as an enzymatic activity, which may be measured using an appropriate enzymatic assay.

As described throughout, the system of the invention is particularly adapted for convenient characterisation of peptides by determination of their amino acid sequence via nucleic acid sequencing in situ, i.e. on the same platform used for screening. Illumina methods for affinity determination are described by Nutiu et al., 2011, Nature Biotechnology, 29, 659-664.

Screening and Selection of Peptides from Libraries

The present invention represents a significant advance in the art for the generation and selection of peptides having desirable properties from libraries (e.g. naïve libraries), and also in drug development, inter alia by allowing screening of peptide libraries for desirable pharmaceutical properties at the same time as characterising the peptides by identification of their nucleic acid sequence that codes for their amino acid sequence.

In accordance with one embodiment of the invention, therefore, in vitro generated nucleic acid libraries encoding a plurality of peptides are synthesised and initially selected for their ability to bind a desired target ligand. In a particularly advantageous method the peptides are synthesised in a CIS in vitro display system, in which each peptide is expressed as a fusion protein to RepA, which binds a target sequence in the nucleic acid (DNA) molecule that encodes the fusion protein, thus forming a complex. In this way, the peptide is linked to the nucleic acid that encoded it (i.e. genotype and phenotype are linked), as a peptide-nucleic acid complex.

The ligand may be a naturally or non-naturally occurring molecule, such as an organic or inorganic small molecule, a carbohydrate, a peptide or a protein sequence. It may be a whole molecule or a part of a larger molecule (e.g. a domain, fragment or epitope of a protein), and may be an intracellular or an extracellular target molecule. In a beneficial embodiment the target is an extracellular ligand, which may be more readily targeted for therapeutic uses.

For in situ sequencing and correlation of genotype (nucleic acid and amino acid sequence) and phenotype (peptide properties), the encoding nucleic acid molecules are immobilised on (associated with or otherwise attached to) a solid support. By way of example, the solid support may be the surface of a glass slide, plate, tube or well; alternatively the solid support may be a bead, such as a magnetic or agarose bead.

The expressed peptide libraries, once generated, are typically incubated with the desired ligand or substrate in order to allow an interaction or reaction to occur, as desired. After a suitable incubation time, unbound ligands and non-associated complexes which remain in free solution/suspension may be removed by aspiration and/or using one or more washing steps with suitable buffers and/or detergents; or by any other means known to the person of skill in the art. A convenient buffer is phosphate-buffered saline (PBS), but other suitable buffers known in the art may also be used.

A particular advantage of the invention, which results from using immobilised library members and related platforms and technology, is that, in contrast to other library screening/selection technologies, only one round of peptide expression and screening/selection may be suitable for identifying library peptides having the desirable properties. For example, where the desired property is a binding affinity for a particular target molecule, a labelled target molecule may be used and allow immediate, localised identification of the useful library member(s).

Any suitable ligand labelling system may be used in accordance with the invention, such as fluorophores, chemiluminescent moieties, radiolabels, antibodies and enzymatic moieties, provided that they may be directly or indirectly detected once bound by the peptide. A suitable labelling moiety may produce an amplified signal (e.g. by catalytic reaction) to allow detection of only a small number of initial positive binding reactions—such systems are particularly useful when the library members are immobilised in a well format that helps to contain/isolate the signalling components. Preferred labels include fluorescent proteins (see e.g. Shaner, (2005), Nature Methods, 2, 905-909).

The invention also encompasses the selection of peptides (or nucleic acids) from a library having more than one desirable property. In this case, more than one round of selection and screening may be conducted sequentially, using different ligands for example.

### Characterisation of Peptides—Binding Affinity

In some embodiments, the desired phenotype to be detected in the screening protocol is binding to a target molecule. Such a desirable interaction can be identified by detecting a binding event and, in some cases, by measuring the binding affinity of the peptide library member for the target molecule.

The selection and screening methods of the invention can thus be applied to the selection of peptides for binding to a desired target ligand. Suitable ligands may include growth factors, receptors, channels, abundant serum proteins, hormones, microbial antigens. Specific examples of potential target ligands include MHC antigens, viral epitopes such as influenza virus, epitopes from parasites such as malaria, or tumour specific antigens.

Binding reactions can be detected and/or affinity measurements can be made using any of the sequencing system instruments described herein or known to the person of skill in the art. The affinity measurement can be made either with or without modification to the analysis instrument, as further described in the non-limiting Examples below.

By way of example, affinity measurements can be taken on a planar surface as used for the Illumina platform. In this regard, the optics of the Illumina systems are based upon the internal reflection illumination of the fluorophores, which excites only fluorophores situated within approximately 100 nm of the flow cell surface. This distance limitation allows the instrument to readily discriminate between fluorophores that are attached (bound/immobilised) to the surface as part of a binding reaction from those that remain free in solution (typically outside of the 100 nm range limit).

Typically, the DNA-protein complexes used for expressing peptide libraries in accordance with the invention have a length of significantly less than 100 nm and so are within the detection range limit of the Illumina assay instrumentation. By way of example, a DNA strand of approximately 1 kb has a length of approximately 3.4 nM. Therefore, bound complexes comprising desired peptide-target molecule binding events will be readily detected (e.g. by way of an appropriate label), whereas target molecules/labels that remain in free solution and generally over 100 nm from the flow cell surface are not detected because they are outside of the detection range.

An advantage of this arrangement is, therefore, that in some embodiments a wash step after performing the screening and/or selection step may not be necessary. In this way the ease and speed of the protocol may be further enhanced. Of course, however, should the background signal be undesirably high at this stage, a wash step may optionally be included to remove unbound signalling molecules as described by Nutiu et al., 2011, Nature Biotechnology, 29, 659-664.

### Nucleic Acids and Peptides

Isolated peptides according to the invention and, where appropriate, the modified or derivatised peptides may be produced by recombinant DNA technology and standard protein expression and purification procedures. Thus, the invention further provides nucleic acid molecules that encode the peptides of the invention as well as their derivatives, and nucleic acid constructs, such as expression vectors that comprise nucleic acids encoding peptides and derivatives according to the invention.

For instance, the DNA encoding the relevant peptide can be inserted into a suitable expression vector (e.g. pGEM®, Promega Corp., USA), where it is operably linked to appropriate expression sequences, and transformed into a suitable host cell for protein expression according to conventional techniques (Sambrook J. et al., Molecular Cloning: a Laboratory Manual, Cold Spring Harbor Press, Cold Spring Harbor, N.Y.). Suitable host cells are those that can be grown in culture and are amenable to transformation with exogenous DNA, including bacteria, fungal cells and cells of higher eukaryotic origin, preferably mammalian cells.

To aid in purifying the peptides of the invention, the peptide (and corresponding nucleic acid) of the invention may include a purification sequence, such as a His-tag. In addition, or alternatively, the peptides may, for example, be grown in fusion with another protein and purified as insoluble inclusion bodies from bacterial cells. This is particularly convenient when the peptide to be synthesised may be toxic to the host cell in which it is to be expressed. Alternatively, peptides may be synthesised in vitro using a suitable in vitro (transcription and) translation system (e.g. the E. coli S30 extract system, Promega corp., USA). By ‘isolated’ as used herein, it does not necessarily mean that the peptide or nucleic acid is ‘pure’; although all levels of purity are encompassed, such as 50% or more, 60% or more, 70% or more, 80% or more, 90% or more, 95% or more and 99% or more.

The term ‘operably linked’, when applied to DNA sequences, for example in an expression vector or construct, indicates that the sequences are arranged so that they function cooperatively in order to achieve their intended purposes, i.e. a promoter sequence allows for initiation of transcription that proceeds through a linked coding sequence as far as the termination sequence.

Having selected and isolated a desired peptide, an additional functional group, such as a therapeutic agent or molecule or label, may then be attached to the peptide by any suitable means. For example, a peptide of the invention may be conjugated to any suitable form of further therapeutic molecule, such has an antibody, enzyme or small chemical compound. This can be particularly useful in applications where the peptide of the invention is capable of targeting or associating with a particular cell or organism, and where the target cell or organism can be treated by that additional conjugated moiety. Peptides of the invention may also be conjugated to a molecule that recruits immune cells of the host, and such conjugates fall within the scope of the invention. Such conjugated peptides may be particularly useful for use as cancer therapeutics.

In another embodiment, the peptide of the invention may be conjugated to an antibody molecule, an antibody fragment (e.g. Fab, F(ab)₂, scFv etc.) or other suitable targeting agent, so that the peptide or its derivative and any further conjugated moieties are targeted to the specific cell population required for a desired treatment or diagnosis.

### Therapeutic and Diagnostic Compositions

A peptide of the invention may be incorporated into a pharmaceutical composition for use in treating an animal, such as a human. A therapeutic peptide of the invention (or derivative thereof) may be used to treat one or more diseases or infections, depending on the target molecule or ligand that was first used to select the particular peptide from the peptide library. Alternatively, a nucleic acid encoding the therapeutic peptide may be inserted into an expression construct and incorporated into pharmaceutical formulations/medicaments for the same purpose.

The therapeutic peptides of the invention may be particularly suitable for the treatment of diseases, conditions and/or infections that can be targeted (and treated) extracellularly, for example, in the circulating blood or lymph of an animal; and also for in vitro and ex vivo applications. Therapeutic nucleic acids of the invention may be particularly suitable for the treatment of diseases, conditions and/or infections that are more preferably targeted (and treated) intracellularly, as well as in vitro and ex vivo applications. As used herein, the terms ‘therapeutic agent’ and ‘active agent’ encompass both peptides and the nucleic acids that encode a therapeutic peptide of the invention.

Therapeutic uses and applications for the peptides and nucleic acids of the invention include: binding partners that prevent protein-protein interactions such as a growth factor binding to a receptor or enzyme or growth factor or cytokine or channel, for example VEGFA binding to its receptor VEGFR2; or indeed binding partners that may agonise a receptor or pathway, such as agonising a GPCR either directly in its peptide binding site or allosterically. Other therapeutic uses for the molecules and compositions of the invention include the treatment of microbial infections and associated conditions, for example, bacterial, viral, fungal or parasitic infection.

In accordance with the invention, the therapeutic peptide or nucleic acid may be manufactured into medicaments or may be formulated into pharmaceutical compositions.

When administered to a subject, a therapeutic agent is suitably administered as a component of a composition that comprises a pharmaceutically acceptable vehicle.

One or more additional pharmaceutically acceptable carrier (such as diluents, adjuvants, excipients or vehicles) may be combined with the therapeutic peptide of the invention in a pharmaceutical composition. Suitable pharmaceutical carriers are described in “Remington's Pharmaceutical Sciences” by E. W. Martin.

Pharmaceutical formulations and compositions of the invention are formulated to conform to regulatory standards and can be administered orally, intravenously, topically, or via other standard routes. The molecules, compounds and compositions of the invention may be administered by any convenient route known in the art.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the invention are formulated in accordance with routine procedures as a pharmaceutical composition adapted for oral administration (more suitably for human beings). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

When the composition is in the form of a tablet or pill, the compositions may be coated to delay disintegration and absorption in the gastrointestinal tract, so as to provide a sustained release of active agent over an extended period of time. Any suitable release formulation known in the art is envisaged.

Additives may be included in the compositions, formulations or medicaments of the invention to enhance cellular uptake of the therapeutic peptide (or derivative) or nucleic acid of the invention, such as the fatty acids oleic acid, linoleic acid and linolenic acid, as is known in the art.

Peptides and nucleic acids of the invention may also be useful in non-pharmaceutical applications, such as in diagnostic tests, imaging, as affinity reagents for purification and as delivery vehicles.

By way of example, peptides of the invention may have utility in various diagnostic applications, such as detection agents for infectious diseases, identification of tumour markers, autoimmune antibodies and biomarkers for therapeutic drug studies.

The invention will now be further illustrated by way of the following non-limiting examples.

### EXAMPLES

Unless otherwise indicated, commercially available reagents and standard techniques in molecular biology and biochemistry were used.

### Materials and Methods

Some of the following procedures used by the Applicant are described in Sambrook, J. et al., 1989 supra.: analysis of restriction enzyme digestion products on agarose gels and preparation of phosphate buffered saline. General purpose reagents were purchased from Sigma-Aldrich Ltd (Poole, Dorset, UK). Oligonucleotides were obtained from Sigma Genosys Ltd (Haverhill, Suffolk, UK) or Genelink Inc., (Hawthorne, N.Y., USA). Amino acids, and S30 extracts were obtained from Promega Ltd (Southampton, Hampshire, UK) or produced according to the methods of Lesley et al. (1991), Journal of Biological Chemistry, 266, 2632-2638. Enzymes and polymerases were obtained from New England Biolabs (NEB) (Hitchin, UK). Sequencing procedures were performed as described in Gupta (2008), Trends Biotechnol., 26(11), 602-611; Shendure & Li (2008), Nature Biotechnol., 26(10), 1135-1145; Rothberg et al., 2011, Nature 475, 348-352; Mardis (2008), Annu. Rev. Genomics Hum. Genet. 9, pp 387-402; Bentley et al., (2008), Nature, 456, 53-59; and Pettersson et al., (2009), Genomics, 93, 105-111; and using the 454 pyrosequencing technique (454 Life Sciences, Roche Diagnostics), the Applied Biosystems (AB) SOLiD™ system, the Ion Torrent sequencing system, the HeliScope™ system, and the Illumina™ system.

Primer, template, peptide and expression construct sequences are shown in Table 1 at the end of the Examples.

### Example 1

### Transcription/Translation on a DNA Template Immobilised Via its 3′ End

In order to demonstrate that proteins can be made on an immobilised template, tac-Cκ-repA-CIS-ori DNA (SEQ ID NO: 1) was amplified by PCR using primers S-R1RecFor and ThioBioXho85 so as to introduce a biotin moiety at its 3′ terminus. The tac-Cκ-repA-CIS-ori DNA template encoded: (i) a tac promoter; (ii) the antibody fragment CK; (iii) the coding region for RepA; (iv) 3′ untranslated control regions, C/S and on (that contain the transcription termination signal and the binding region for RepA).

The PCR conditions to generate the biotinylated DNA construct tac-Cκ-RepA-CIS-ori-bio (SEQ ID NO: 4) were as follows for 8× 50 μl volume PCR reactions:

| | | |
|---|---|---|
| tac-C_{κ}-repA-CIS-ori (200 ng/μl) | 1 | μl |
| ThermoPol buffer (10x) | 40 | μl |
| dNTPs (10 mM) | 8 | μl |
| S-R1RecFor (#583) (SEQ ID NO. 2) (10 μM) | 8 | μl |
| ThioBioXho85 (#514) (SEQ ID NO. 3) (10 μM) | 8 | μl |
| Taq polymerase (NEB) (5 u/μl) | 4 | μl |
| H₂O | 331 | μl |

The PCR conditions used were 95° C. for 2 minutes followed by 30 cycles at 95° C. for 30 seconds, 60° C. for 30 seconds and 72° C. for 1 minute in a Techne TC3000 PCR machine. The resulting biotinylated DNA was then purified using Promega Wizard columns and eluted in 50 μl Elution Buffer (EB; Qiagen, Crawley, West Sussex, UK). The concentration of the DNA was measured by UV spectroscopy and 2 μg tac-Cκ-repA-C/S-ori-bio DNA was then subjected to a transcription-translation reaction as described below (without washing of beads for the ‘In Solution’ procedure).

For comparative purposes the transcription and translation procedure was performed both in ‘Solid Phase’ and ‘In Solution’. For the ‘Solid Phase’ procedure the template DNA was first immobilised onto 100 μl streptavidin microbeads (M280, Invitrogen) before carrying out the transcription and translation; whereas the ‘In Solution’ procedure was performed on free template DNA (in the absence of beads). Following the transcription and translation procedure the ‘In Solution’ reaction mixture was also then captured on beads to immobilise the nucleic acid template. Thereafter, both ‘Solid Phase’ and ‘In Solution’ samples were treated in the same manner.

Immobilisation of template DNA on beads was performed by incubation of the biotinylated tac-Cκ-repA-CIS-ori-bio template with 100 μl streptavidin microbeads for 10 minutes in PBS whilst rotating of the beads. Following the incubation, the beads were captured against the side of the tube using a magnet. The beads were washed three times with 1 ml PBS containing 0.1% Tween-20 (polysorbate 20; PBST) and washed twice further with 1 ml PBS.

For the Solid Phase procedure the beads were then resuspended in 10 μl H₂O and 40 μl of an in vitro transcription/translation (ITT) mixture was added. The ITT mixture contained 15 μl S30 lysate and 20 μl 2.5× buffer and 5 μl amino acid mixture (Lesley et al. 1991, Journal of Biological Chemistry, 266, 2632-2638; Zubay et al. 1973, Annual Review of Genetics 7, 267-287). The transcription/translation reaction was incubated for 1 hour at 30° C., following which 450 μl Block Buffer (PBST containing 2% bovine serum albumin (Sigma), 1 mg/ml heparin (Sigma), 100 μg/ml herring sperm DNA (Promega)) was added. The beads were washed three times with 1 ml PBST and twice with PBS before being resuspended in 200 μl goat anti-human Cκ-HRP (horseradish peroxidise; Serotec Ltd., Toronto, Canada), diluted 1:1,000 in Block Buffer, and incubated whilst rotating for 50 min. at room temperature. This was again washed with three washes with 1 ml PBST and two with 1 ml PBS. The last wash was removed and the beads were resuspended in the 75 μl HRP reagent tetramethyl benzidine (TMB; TrueBlue; Kirkegaard & Perry Laboratories, Inc, Gaithersburg, Md.), and the reaction terminated after a suitable time by the addition of 75 μl 0.5 M H₂SO₄.

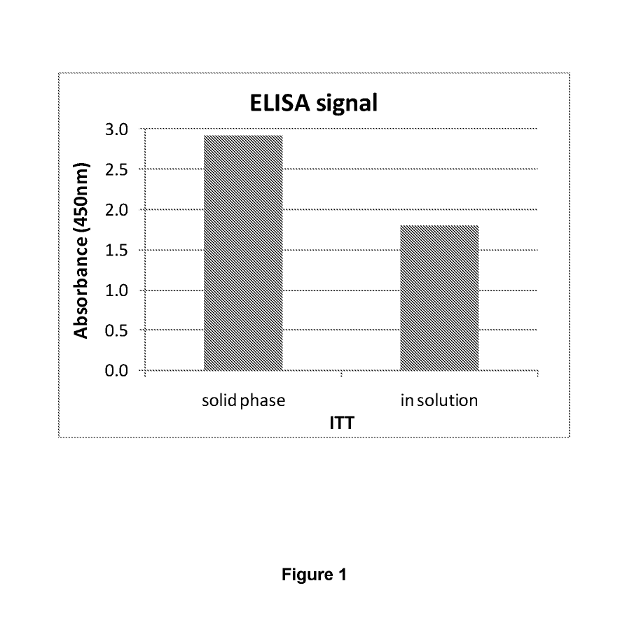

100 μl of each resultant solution was transferred to a flat-bottomed 96-well microtitre plate and the absorbance at 450 nm was measured in a plate reader to determine the amount of expressed protein that was immobilised on microbeads via conjugation of the encoding nucleic acid template. The results of the ELISA assay are shown in FIG. 1. This data illustrates that proteins are expressed and captured on beads via each of the ‘Solid Phase’ and ‘In Solution’ procedures. Although the ELISA signal from the ‘Solid Phase’ test is higher than that of the ‘In Solution’ experiment in this study, the general result may not be statistically relevant.

### Example 2

### Transcription/Translation on a DNA Template Immobilised Via its 5′ End

Other templates encoding a V5 peptide, were prepared by PCR similarly to that described in Example 1, except a tac-V5-repA-CIS-ori (SEQ ID NO: 5) template was used and amplified by 25 cycles of PCR using: primers #144-tach (SEQ ID NO: 8) and #514-ThioBioXho85 (SEQ ID NO: 3) to produce template tac-V5-repA-CIS-ori-bio (SEQ ID NO: 6) having a biotin moiety near its 3′ end; and with primers #472-R1 RecForbio (SEQ ID NO:9) and #85-Orirev (SEQ ID NO: 10) to produce template bio-tac-V5-repA-(SEQ ID NO: 7) having a biotin moiety attached at its terminus. The control tac-V5-repA-CIS-ori (SEQ ID NO: 5) was not biotinylated.

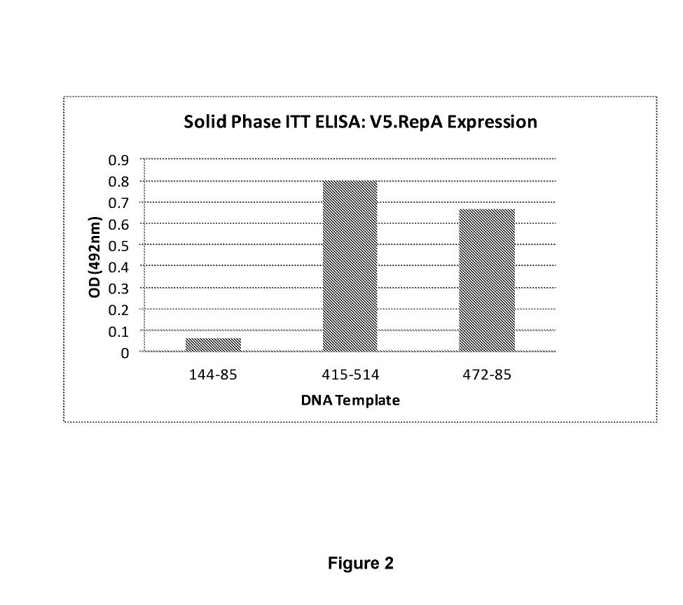

The amplified DNA was purified using QIAquick columns and the DNA eluted in 50 μl EB. 10 μg of tac-V5-repA-CIS-ori-bio (144-514; FIG. 2); tac-V5-repA-CIS-ori (V5.RepA 144-85; FIG. 2); bio-tac-V5-repA-CIS-ori (472-85; FIG. 2) made up to 400 μl with water were added to 100 μl M280 streptavidin beads (prewashed twice with 400 μl Invitrogen Binding Buffer; Invitrogen, Life Technologies, Paisley, UK) in 400 μl Invitrogen Binding Buffer (Invitrogen). The mixture was left rotating for 3 hours at room temperature, and the beads were then washed twice with 400 μl Invitrogen wash buffer and once with 400 μl H₂O. The beads were resuspended in 50 μl H₂O and then an ITT was performed as described above, but using 200 μl of bacterial buffer and lysate mix per 10 μg DNA sample. The lysate and buffer were prepared without any DTT. The mixture was incubated for 1 hour 37° C. in a waterbath and then incubated on ice for 40 mins. 450 μl Block Buffer was added and incubated for 20 min. on ice. The beads were then washed three times with 750 μl PBST and once with 750 μl PBS. The beads were then resuspended in 1 ml anti-V5-HRP (diluted 1:1000 in 2% BSA; Abcam, Cambridge, UK) and left rotating for 50 min. at room temperature. The beads were again washed three times with 750 μl PBST and once with 750 μl PBS and finally resuspended in 100 μl TMB. The reaction was terminated with 100 μl 0.5M H₂SO₄ and 150 μl of the solution transferred to a flat bottomed 96-well microtitre plate and read at 492 nm in a plate reader. The results are displayed in FIG. 2. As illustrated, the constructs that were capable of being immobilised on the solid support gave relatively high ELISA signals, indicating that the peptide was expressed and captured on the support via cis-binding back to its encoding DNA template. By contrast the control experiment in which template was missing a biotin moiety and so was unable to be immobilised on the solid support did not produce a notable ELISA signal, indicating that V5 peptide was not captured on the plate of this sample. Imobilisation via the 3′ end of the template resulted in a slightly higher ELISA signal, but it is not known whether this is statistically significant.

### Example 3

### CIS Display of Template DNA Immobilised on a Planar Surface

Both tac-Cκ-repA-CIS-ori-bio (SEQ ID NO: 4) and tac-V5-repA-CIS-ori-bio (SEQ ID NO: 6) were prepared by PCR as described above. 2 μg each template DNA was added separately to 50 μl ITT reactions to create Cκ-RepA protein-DNA and V5-RepA protein-DNA nucleic acid-peptide fusions. Two 25 μl aliquots of each mixture was then added to wells of a streptavidin coated microtitre plate that had been previously blocked for 1 hour with 250 μl Block Buffer and washed twice with 200 μl PBS. After addition of the ITT mixture the plates was incubated for 10 min., washed three times with 200 μl PBST, and then washed twice further with 200 μl PBS.

100 μl anti-Cκ-HRP or anti-VS-HRP (1:1,000 in PBS containing 2% BSA) was added to each sample and incubated at room temperature, followed by three washes of 200 μl PBST and two washes with 200 μl PBS. After removal of the last wash volume, 50 μl of BM Chemilluminescence ELISA substrate (Roche, Burgess Hill, UK) was added according to manufacturer's instructions, using 100 parts of Substrate Reagent A buffered solution that contains luminol/4-iodophenol to 1 part of Substrate Reagent B (buffered solution that contains a stabilised form of H₂O₂). The signal was detected using a Perkin Elmer Envision plate reader. The results, not shown, demonstrate that Cκ-HRP and V5-HRP are expressed from immobilised template DNA and fold sufficiently to be recognised by the anti-Cκ-HRP and anti-VS-HRP antibodies respectively.

### Example 4

### Bridge Amplification and Sequencing

### Preparation of DNA

The following procedures were performed to produce a DNA template for bridge amplification and sequencing as described in U.S. Pat. No. 7,232,656, Bentley et al., 2008, Nature. 456, 53-59. A degenerate codon library was designed that could be displayed in fusion with RepA and detected using a conjugated anti-FLAG antibody such as anti-FLAG-M2 Cy3 (Sigma Aldrich) or DYKDDDDK Tag Alexa Fluor® 647 conjugated antibody (New England Biolabs, NEB).

PCR Reactions were Set Up as Follows:

| 10 × 50 μl reactions | | |
|---|---|---|
| 1steprepA template (SEQ ID NO. 11) (200 ng/μl) | 100 | ng |
| Standard buffer (10x) | 75 | μl |
| dNTPs (10 mM) | 10 | μl |
| flag-libfor (SEQ ID NO. 12) (10 μM) | 10 | μl |
| #85-Orirev (SEQ ID NO. 10) (10 μM) | 10 | μl |
| Taq polymerase (NEB) (5 u/μl) | 5 | μl |

| | |
|---|---|
| H₂O | up to 500 μl |

The resulting flaglib-repA-CIS-ori DNA (SEQ ID NO: 13) was amplified in a thermocycler using primers 131-mer (SEQ ID NO: 14) and #85-Orirev (SEQ ID NO: 10) using the following protocol: 95° C. for 2 minutes, and then 25 cycles at 95° C. for 30 seconds, 55° C. for 30 seconds, 68° C. for 1 minute, followed by a final extension reaction at 68° C. for 5 minutes; to produce the product tac-flaglib-repA-CIS-ori (SEQ ID NO: 15) in 20× 50 μl reactions (see below). The DNA was then purified using a QIAquick PCR cleanup kit (Qiagen, Crawley, West Sussex, UK) according to the manufacturer's instructions.

| | | |
|---|---|---|
| flaglib-repA-CIS-ori | 5 | μg |
| Standard buffer (10x) | 150 | μl |
| dNTPs (10 mM) | 20 | μl |
| 131-mer (10 μM) | 20 | μl |
| #85-Orirev (10 μM) | 20 | μl |
| Taq polymerase (NEB) (5 u/μl) | 10 | μl |

| | |
|---|---|
| H₂O | up to 1000 μl |

Purified DNA was then amplified with 6 to 18 cycles of PCR using the Phusion High-Fidelity system (New England Biolabs) and primers C (SEQ ID NO: 18) and D (SEQ ID NO: 19) to produce a template tac-flaglib-illmunadapt (SEQ ID NO: 38) suitable for ‘paired-reads’. However, alternatively, primers for single reads A (SEQ ID NO: 16) and B (SEQ ID NO: 17) could be used. Samples were diluted to a concentration of 10 nM in 10 mM Tris pH 8.5 and 0.1% Tween 20 prior to cluster formation (as described below).

### Preparation of Flowcells

Glass 8-channel flow cells (Silex Microsystems, Sweden) were thoroughly washed and then coated for 90 min at 20° C. with 2% acrylamide containing approximately 3.9 mg/ml N-(5-bromoacetamidylpentyl) acrylamide, 0.85 mg/ml tetramethylethylenediamine (TEMED) and 0.48 mg/ml potassium persulfate (K₂S₂O₈). Flow cell channels were rinsed thoroughly before further use. The coated surface was then functionalised by reaction for 1 hour at 50° C. with a mixture containing 0.5 μM each of two priming oligonucleotides (oligos C′ and D′, SEQ ID NO: 20 and SEQ ID NO: 21, respectively) in 10 mM potassium phosphate buffer pH 7. Flowcells contained the two oligonucleotides immobilised on the surface in a ratio C′:D′ of 1:1. Grafted flow cells were stored in 5×SSC until required.

### Cluster Creation

Cluster creation was carried out using an Illumina Cluster Station. To obtain single stranded templates, DNA was first denatured in NaOH (to a final concentration of 0.1 M) and subsequently diluted in cold (4° C.) hybridisation buffer (5×SSC+0.05% Tween 20) to working concentrations of 2 to 4 μM, depending on the desired cluster density/tile.

85 μl of each sample was primed through each lane of a flowcell at 96° C. (60 μl/min). The temperature was then slowly decreased to 40° C. at a rate of 0.05° C./sec to enable annealing of tac-flaglib-illumadapt DNA to complementary oligonucleotides (C′ and D′) immobilised on the flowcell surface. Oligos hybridised to template strands were extended using Taq polymerase to generate a surface-bound complement of the template strand. The samples were then denatured using formamide to remove the initial seeded template. The remaining immobilised single stranded copy was the starting point for cluster creation—it being able to anneal to a close-by complementary immobilised oligo (the other of C′ or D′, respectively) for amplification of the extended template.

Clusters were created/amplified under isothermal conditions at 60° C. for 35 cycles using Bst polymerase for extension and formamide for denaturation during each cycle. Clusters were washed with storage buffer (5×SSC) and either stored at 4° C. or used directly.

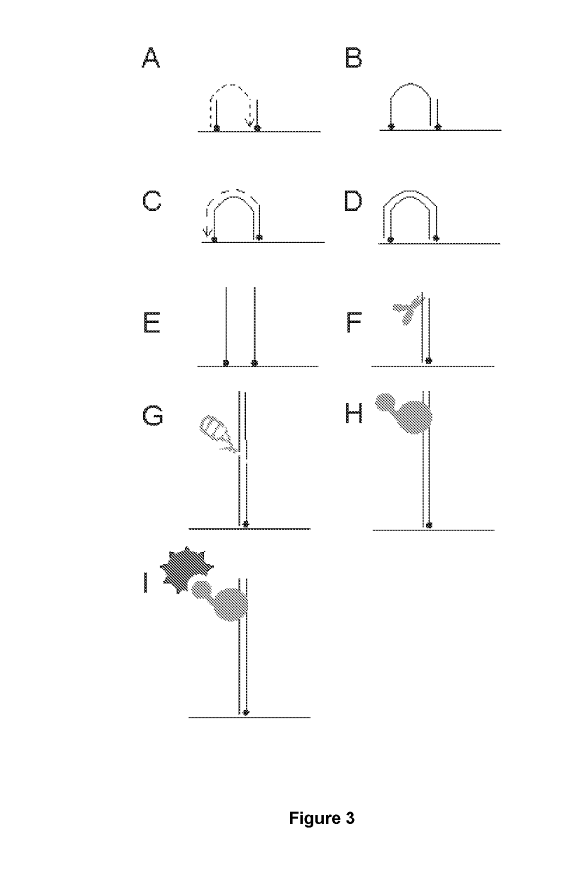

FIG. 3 (A to E) illustrates an exemplary procedure for cluster creation and sequencing.

### Processing of Clusters for Sequencing Experiments

Linearisation of surface immobilised oligo C′ to retain strand ‘1’ of each cluster was achieved by incubation with USER enzyme mixture (Illumina) to treat the deoxyuridine-containing oligonucleotide. After blocking, clusters were denatured with 0.1 M NaOH prior to hybridisation of the Read 1 Specific Sequencing Primer (5′-ACACTCTTTCCCTACACGACGCTCTTCCGATCT-3′; SEQ ID NO: 22). Processed flowcells were transferred to the Illumina Genome Analyser for sequencing.

### Sequencing on the Genome Analyser.

All sequencing runs were performed as described in the Illumina Genome Analyser operating manual. Flowcells were sequenced using standard recipes (see User Guide) in order to generate 25 and 35 base single and paired reads.

### Example 5

### CIS Display In Situ in the Flow Cell

Cleavage of DNA fragment and ligation of repA-CIS-ori DNA Following the successful completion of the sequencing on the Genome Analyser, flowcells, clusters were denatured with 0.1 M NaOH to remove the products of Read 1. Clusters were then 3′-dephosphorylated using T4 polynucleotide kinase, and the strand that had been linearised as part of the sequencing read was re-synthesised isothermally as previously described for cluster creation (FIG. 3E).

The dsDNA was next treated with BsaI-HF enzyme in 1× NEBuffer 4, supplemented with 100 μg/ml BSA (NEB) by flowing the enzyme into the cell and incubating at 37° C. for 1 hour to create a sticky-end single stranded overhang. The flow cell was then washed with 1× SSC containing 0.05% Tween-20 (FIG. 3F).

1steprepA (SEQ ID NO: 11) DNA was amplified with Bsa-repfor (5′-aaaGGTCTCccaactgatcttcaccaaacgtattacc-3′; SEQ ID NO: 23) and #85-Orirev, as described above using PCR, to create a BsaI site at the 5′ end of the repA sequence bsarepA-CIS-ori (SEQ ID NO: 39). Following column purification, 10 μg of pure bsarepA-CIS-ori were digested with BsaI-HF enzyme (NEB) in 1× NEBuffer 4 (NEB), supplemented with 100 μg/ml BSA (NEB) for 1 hour at 37° C. The DNA was subsequently purified through agarose in order to remove the small 5′ fragment and retain the digested bsarepA-CIS-ori region.

Ligation of Cleaved bsarepA-CIS-Ori

5 pmol of BsaI digested bsarepA-CIS-ori was diluted into a ligase mix containing 4,000U T4 DNA ligase (NEB), 1× T4 DNA Ligase Reaction Buffer (NEB) and flowed into the flow cell and incubated for 1 hour at 30° C. This ligates the repA sequence containing a complementary single stranded overhang to the DNA attached to the surface of the flow cell. The flow cell was then rinsed with 1× SSC containing 0.05% Tween-20 followed by a wash with 10 mM Tris pH 7.5 in preparation for transcription and translation (see FIG. 3G)

ITT In Situ within the Flow Cell

An ITT mixture was prepared as described in Example 1 above and passed onto the flow cell. The cell was incubated for 1 hour at 30° C. before being washed with PBST and then further with PBS. This enabled the peptide-RepA fusions to be expressed and bind to their own DNA template on the surface of the array (FIG. 3H). The surface was then blocked with Block Buffer and incubated for 20 min. at room temperature and washed with PBST and then with PBS. A solution of anti-DYKDDDDK Tag Alexa Fluor® 647 conjugated antibody (NEB; 1:500 or 1:1000 in PBS containing 2% BSA) was added and incubated at room temperature for 1 hour. This was again washed with PBST and then with PBS (FIG. 3I).

The fluorescent signal corresponding to binding of the antibody to the FLAG epitope present in library peptides immobilised on the flow cell was measured by laser excitation at 630 nm or 650 nm with monitoring the emission at 668 nm.

### Example 6

### Alternative Cluster Creation Method

An alternative to the Cluster Creation method described in Example 4 is anticipated so that full-length DNA templates can be used without digestion and ligation of a universal sequence portion (e.g. containing the cis-binding agent, repA) onto the tac-flaglib-illumadapter fragments. In this Example, cluster creation was carried out using an Illumina Cluster Station.

To obtain single stranded templates, adapted full length DNA (tac-flaglib-repA-CIS-on) was amplified using oligonucleotides Primer D and Primer E

SEQ ID NO: 24)
(5′-
AATGATACGGCGACCACCGAGATCTACACTCTTTCCCTACACGACGCTCT
TCCGATCTCtgcatatctgtctgtccacagg-3′;

using the conditions described above for PCR with primers C and D, with Primer E replacing Primer C to create tac-flaglib-repA-CIS-ori-illumadapt (SEQ ID NO: 40) over 25 cycles of amplification.

The DNA was purified and eluted in 10 mM Tris-CI, pH 8.5 followed by denaturation in NaOH (to a final concentration of 0.1 M) and subsequent dilution in cold (4° C.) hybridisation buffer (5×SSC+0.05% Tween 20) to working concentrations of 0.2 to 4 μM, depending on the desired cluster density/tile. A greater dilution of the template concentration would allow the longer DNA template to form discreet clusters following amplification.

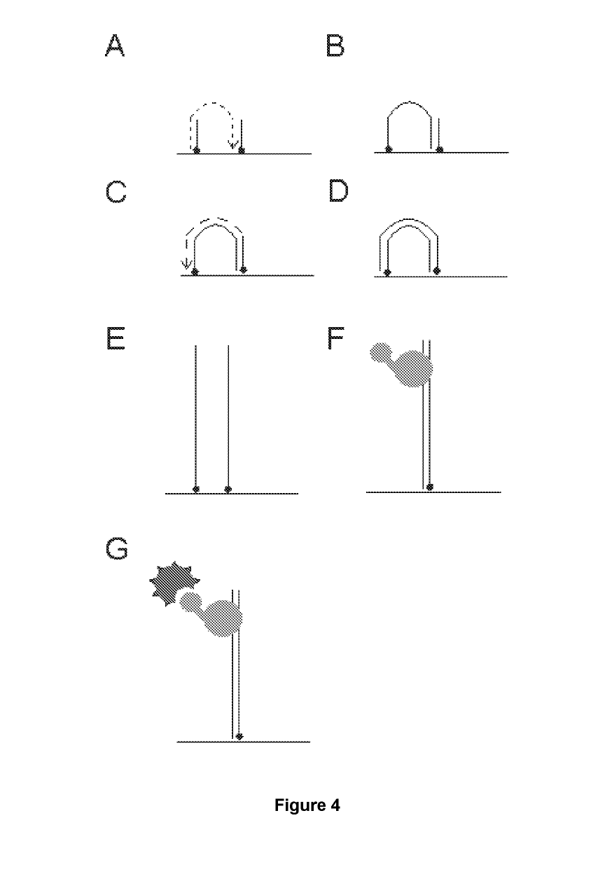

Sequencing was as described above using primer D and cleavage of DNA fragments with BsaI and ligation of repA-CIS-ori DNA were not necessary. The ITT process was carried out as described above. However, treatment of the DNA template to reconstitute the double-stranded nature of the DNA template with Bst polymerase was still required prior to ITT. This exemplary method is illustrated schematically in FIG. 4.

### Example 7

### DNA Capture on Microparticles, Emulsion PCR, Sequencing and CIS Display

A comparable procedure was carried out to that described in Example 5 above, but using the Roche 454 sequencing system approach as described in detail in Margulies et al., (2005), Nature, 437(15), 376-380 and accompanying supplemental materials.

### Emulsion PCR Methods

PCR products from a polyclonal mixture of DNA templates from a tac-flaglib-RepA-CIS-ori template were generated by PCR amplification with primers containing the sequences for the standard 454 adapter sequences. The forward primer Adapter A (SEQ ID NO: 25) anneals to the tac promoter sequence, and the reverse primer Adapter B (SEQ ID NO: 26) anneals at the 3′ end of ori.

These sequences contained a four base, non-palindromic sequencing ‘key’ comprised of one of each deoxyribonucleotide (e.g. TCAG). The tac-flaglib-repA-CIS-ori-454adapt DNA product (SEQ ID NO: 27) was purified through QIAquick columns and eluted into 50 μl EB Buffer.

100 μl of stock M-270 streptavidin beads (Dynal, Oslo, Norway) were washed twice in a 1.5 ml microcentrifuge tube with 200 μl of 1× B&W Buffer (5 mM Tris-HCl, pH 7.5, 0.5 mM EDTA, 1 M NaCl) by vortexing the beads in the wash solution, immobilising the beads with the Magnetic Particle Concentrator (MPC; Dynal), drawing the solution off from the immobilised beads and repeating. After the second wash, the beads were resuspended in 100 μl of 2× Binding and Wash (B&W) Buffer (10 mM Tris-HCl, pH 7.5, 1 mM EDTA, 2 M NaCl), to which the entire 80 μl of the amplified tac-flaglib-repA-CIS-ori-454adapt and 20 μl of Molecular Biology Grade water were then added. The sample was then mixed by vortexing and placed on a horizontal tube rotator for 20 minutes at room temperature. The bead mixture was then washed twice with 200 μl of 1× B&W Buffer, then twice with 200 μl of Molecular Biology Grade water.

### Preparation of Single Stranded DNA

The final water wash was removed from the bead pack using the MPC, and 250 μl of Melt Solution (100 mM NaCl, 125 mM NaOH) was added. The beads were re-suspended with thorough mixing in the melt solution and the bead suspension incubated for 10 minutes at room temperature on a tube rotator.

In a separate 1.5 ml centrifuge tube, 1,250 μl of buffer PB (from the QiaQuick PCR Purification Kit) was neutralised by addition of 9 μl 20% aqueous acetic acid. Using the Dynal MPC, the beads in the melt solution were pelleted; the 250 μl of supernatant (containing the now single-stranded library) was carefully decanted and then transferred to the tube of freshly-prepared neutralised buffer PB.

The 1.5 ml of neutralised, single-stranded library was concentrated over a single column from a MinElute PCR Purification Kit (Qiagen, Crawley, West Sussex, UK), and warmed to room temperature prior to use. The sample was loaded and concentrated in two 750 μl aliquots. Concentration of each aliquot was conducted according to the manufacturer's instructions for spin columns using a microcentrifuge, with the following modifications: the dry spin after the Buffer PE spin was extended to 2 minutes (rather than 1 minute) to ensure complete removal of the ethanol, and the single-stranded library sample was eluted in 15 μl of Buffer EB (Qiagen) at 55° C.

The quantity and quality of the resultant single-stranded DNA library was assessed with the Agilent 2100 and a fluorescent plate reader. As the library consisted of single-stranded DNA, an RNA Pico 6000 Lab-Chip for the Agilent 2100 was used and prepared according to the manufacturer's guidelines. Triplicate 1 μl aliquots were analysed, and the mean value reported by the Agilent analysis software was used to estimate the DNA concentration. The final library concentration was typically in excess of 10e8 molecules/μl. The library samples were stored in concentrated form at −20° C. until needed.

### Preparation of DNA Capture Beads

Packed beads from a 1 ml N-hydroxysuccinimide ester (NHS)-activated Sepharose HP affinity column (Amersham Biosciences, Piscataway, N.J.) were removed from the column and activated as described in the product literature (Amersham Pharmacia Protocol #71700600AP). 25 μl of a 1 mM amine-labelled HEG capture primer (5′-Amine-3 sequential 18-atom hexaethyleneglycol spacers CCTATCCCCTGTGTGCCTTG-3′; SEQ ID NO: 28; IDT Technologies, Coralville, Iowa, USA) in 20 mM phosphate buffer, pH 8.0, was bound to the beads, after which beads having a diameter in the range of approximately 25 to 36 μm were selected by serial passage through 36 and 25 μm pore filter mesh sections (Sefar America, Depew, N.Y., USA). DNA capture beads that passed through the first filter, but were retained by the second were collected in bead storage buffer (50 mM Tris, 0.02% Tween, 0.02% sodium azide, pH 8), quantitated with a Multisizer 3 Coulter Counter (Beckman Coulter, Fullerton, Calif., USA) and stored at 4° C. until needed.

### Binding Template Species to DNA Capture Beads

Template molecules were annealed to complementary primers on the DNA Capture beads in a UV-treated hood. 1,500,000 DNA capture beads suspended in bead storage buffer were transferred to a 200 μl PCR tube, centrifuged in a microfuge for 10 seconds, and the tube was then rotated 180° and spun for an additional 10 seconds to ensure even pellet formation. The supernatant was removed, and the beads washed with 200 μl of Annealing Buffer (20 mM Tris, pH 7.5 and 5 mM magnesium acetate), vortexed for 5 seconds to resuspend the beads, and pelleted as above. All but approximately 10 μl of the supernatant above the beads was removed, and an additional 200 μl of Annealing Buffer was added. The beads were vortexed again for 5 seconds, allowed to sit for 1 minute, then pelleted as above. This time, all but about 10 μl of supernatant was discarded, and 1.2 μl of 2× 10e7 molecules per μl template library was added to the beads. The tube was vortexed for 5 seconds to mix the contents, after which the templates were annealed to the beads in a controlled denaturation/annealing program performed in an MJ thermocycler (5 minutes at 80° C., followed by a decrease by 0.1° C./sec to 70° C.; 1 minute at 70° C., followed by a decrease by 0.1° C./sec to 60° C.; hold at 60° C. for 1 minute, followed by a decrease by 0.1° C./sec to 50° C.; hold at 50° C. for 1 minute, followed by a decrease by 0.1° C./sec to 20° C.; hold at 20° C.). Upon completion of the annealing process the beads were stored on ice until needed.

### PCR Reaction Mix Preparation and Formulation

The PCR reaction mix was prepared in a UV-treated hood located in a PCR clean room. For each 1,500,000 bead emulsion PCR reaction, 225 μl of reaction mix containing 1× Platinum HiFi Buffer (Invitrogen), 1 mM dNTPs (Pierce), 2.5 mM MgSO₄ (Invitrogen), 0.1% acetylated, molecular biology grade BSA (Sigma, St. Louis, Mo.), 0.01% Tween-80 (Acros Organics, Morris Plains, N.J.), 0.003 U/μl thermostable pyrophosphatase (NEB), 0.625 μM 454 Seq Forward (5′-CCATCTCATCCCTGCGTGTC-3′; SEQ ID NO: 29) and 0.039 μM 454 Seq Reverse primers (5′-CCTATCCCCTGTGTGCCTTG-3′; SEQ ID NO: 30; IDT Technologies) and 0.15 U/μl Platinum Hi-Fi Taq Polymerase (Invitrogen), was prepared in a 1.5 ml tube.

25 μl of the reaction mix was removed and stored in an individual 200 μl PCR tube for use as a negative control. Both the reaction mix and negative controls were stored on ice until needed. Additionally, 240 μl of mock amplification mix containing 1× Platinum HiFi Buffer (Invitrogen), 2.5 mM MgSO₄ (Invitrogen), and 0.1% BSA, 0.01% Tween for every emulsion was prepared in a 1.5 ml tube, and similarly stored at room temperature until needed.

### Emulsification and Amplification

The emulsification process creates a heat-stable water-in-oil emulsion with approximately 1,000 discrete PCR microreactors per microliter, which serve as a matrix for single molecule, clonal amplification of the individual molecules of the target library.

The reaction mixture and DNA capture beads for a single reaction were emulsified in the following manner: in a UV-treated hood, 160 μl of PCR solution was added to the tube containing the 1,500,000 DNA capture beads. The beads were resuspended through repeated pipette action, after which the PCR-bead mixture was permitted to sit at room temperature for at least 2 minutes, allowing the beads to equilibrate with the PCR solution. Meanwhile, 400 μl of Emulsion Oil containing 40% w/w DC 5225C Formulation Aid (Dow Chemical Co., Midland, Mich.), 30% w/w DC 749 Fluid (Dow Chemical Co.), and 30% w/w Ar20 Silicone Oil (Sigma), was aliquoted into a flat-topped 2 ml centrifuge tube (Dot Scientific, Burton, Mich.). The 240 μl of mock amplification mix was then added to 400 μl of emulsion oil, and the tube capped securely and placed in a 24 well TissueLyser Adaptor (Qiagen) of a TissueLyser MM300 (Retsch GmbH & Co. KG, Haan, Germany). The emulsion was homogenised for 5 minutes at 25 oscillations/sec to generate the extremely small emulsions, or ‘microfines’, that confer additional stability to the reaction.

The combined beads and PCR reaction mix were briefly vortexed and allowed to equilibrate for 2 minutes. After the microfines had been formed, the amplification mix, templates and DNA capture beads were added to the emulsified material. The Tissue-Lyser speed was reduced to 15 oscillations/sec and the reaction mix homogenised for 5 minutes. The lower homogenisation speed created water droplets in the oil mix with an average diameter of 100 to 150 μm, sufficiently large to contain DNA capture beads and amplification mix.

The total volume of the emulsion (approximately 800 μl) was contained in one 2 ml flat-topped centrifuge tube. Next, the emulsion was aliquoted into 7 or 8 separate PCR tubes each containing roughly 100 μl. The tubes were sealed and placed in a MJ thermocycler along with the 25 μl negative control made previously. The following PCR cycle times were used: 1× 4 minutes at 94° C. (Hotstart Initiation); 40× 30 seconds at 94° C., 60 seconds at 58° C., 90 seconds at 68° C. (Amplification); 13× 30 seconds at 94° C., 360 seconds at 58° C. (Hybridization Extension). After completion of the PCR program, the reactions were removed and the emulsions either broken immediately (as described below) or the reactions stored at 10° C. for up to 16 hours prior to initiating the breaking process.

### Breaking the Emulsion and Recovery of Beads

50 μl of isopropyl alcohol (Fisher) was added to each PCR tube containing the emulsion of amplified material, and vortexed for 10 seconds to lower the viscosity of the emulsion. The tubes were centrifuged for several seconds in a microcentrifuge to remove any emulsified material trapped in the tube cap. The emulsion-isopropyl alcohol mix was withdrawn from each tube into a 10 ml BD Disposable Syringe (Fisher Scientific) fitted with a blunt 16 gauge blunt needle (Brico Medical Supplies, Metuchen, N.J.). An additional 50 μl of isopropyl alcohol were added to each PCR tube, vortexed, centrifuged as before, and added to the contents of the syringe. The volume inside the syringe was increased to 9 ml with isopropyl alcohol, after which the syringe was inverted and 1 ml of air was drawn into the syringe to facilitate mixing the isopropanol and emulsion.

The blunt needle was then removed, and a 25 mm Swinlock filter holder (Whatman, Middlesex, United Kingdom) containing 15 μm pore Nitex Sieving Fabric (Sefar America, Depew, N.Y., USA) attached to the syringe luer, and the blunt needle affixed to the opposite side of the Swinlock unit. The contents of the syringe were gently but completely expelled through the Swinlock filter unit and needle into a waste container containing bleach. 6 ml of fresh isopropyl alcohol was drawn back into the syringe through the blunt needle and Swinlock filter unit, and the syringe inverted 10 times to mix the isopropyl alcohol, beads and remaining emulsion components. The contents of the syringe were again expelled into a waste container, and the wash process repeated twice with 6 ml of additional isopropyl alcohol in each wash. The wash step was repeated with 6 ml 80% Ethanol/1× Annealing Buffer (80% Ethanol, 20 mM Tris-HCl, pH 7.6, 5 mM magnesium acetate). The beads were then washed with 6 ml 1× Annealing Buffer with 0.1% Tween (0.1% Tween-20, 20 mM Tris-HCl, pH 7.6, 5 mM Magnesium Acetate), followed by a 6 ml wash with molecular biology grade pure water.

After expelling the final wash into the waste container, 1.5 ml of 1 mM EDTA was drawn into the syringe, and the Swinlock filter unit removed and set aside. The contents of the syringe were serially transferred into a 1.5 ml centrifuge tube. The tube was periodically centrifuged for 20 seconds in a minifuge to pellet the beads and the supernatant removed, after which the remaining contents of the syringe were added to the centrifuge tube. The Swinlock unit was reattached to the filter and 1.5 ml of EDTA drawn into the syringe. The Swinlock filter was removed for the final time, and the beads and EDTA added to the centrifuge tube, pelleting the beads and removing the supernatant as necessary.

### Second-Strand Removal

Amplified DNA, immobilised on the capture beads, was rendered single stranded by removal of the secondary strand through incubation in a basic ‘melt’ solution. 1 ml of freshly prepared Melting Solution (0.125 M NaOH, 0.2 M NaCl) was added to the beads, the pellet resuspended by vortexing at a medium setting for 2 seconds, and the tube placed in a Thermolyne LabQuake tube roller for 3 minutes. The beads were then pelleted as above, and the supernatant carefully removed and discarded. The residual melt solution was then diluted by the addition of 1 ml Annealing Buffer (20 mM Tris-Acetate, pH 7.6, 5 mM magnesium acetate), after which the beads were vortexed at medium speed for 2 seconds, and the beads pelleted, and supernatant removed as before. The Annealing Buffer wash was repeated, except that only 800 μl of the Annealing Buffer was removed after centrifugation. The beads and remaining Annealing Buffer were transferred to a 0.2 ml PCR tube, and either used immediately or stored at 4° C. for up to 48 hours before continuing with the subsequent enrichment process.

### Enrichment of Beads

Up to this point the bead mass was comprised of both beads with amplified, immobilised DNA strands, and null beads with no amplified product. Therefore, an enrichment process was utilised to selectively capture beads with sequenceable amounts of template DNA while rejecting the null beads.

The beads having single-stranded DNA from the previous step were pelleted by 10 second centrifugation in a bench-top mini centrifuge, after which the tube was rotated 180° and spun for an additional 10 seconds to ensure even pellet formation. As much supernatant as possible was then removed without disturbing the beads. 15 μl of Annealing Buffer was added to the beads, followed by 2 μl of 100 μM biotinylated, 40 base HEG enrichment primer (5′ Biotin—18-atom hexa-ethyleneglycol spacer (C₁₂H₂₆O₇)-

SEQ ID NO: 31
CCATCTCATCCCTGCGTGTCCCATCTGTTCCCTCCCTGTC-3′;;

(IDT Technologies), complementary to the combined amplification and sequencing sites (each 20 bases in length) on the 3′-end of the bead-immobilised template. The solution was mixed by vortexing at a medium setting for 2 seconds, and the enrichment primers annealed to the immobilised DNA strands using a controlled denaturation/annealing program in an MJ thermocycler. (30 seconds at 65° C., decrease by 0.1° C./sec to 58° C., 90 seconds at 58° C., and a 10° C. hold).

While the primers were annealing, a stock solution of SeraMag-30 magnetic streptavidin beads (Seradyn, Indianapolis, Ind., USA) was resuspended by gentle swirling, and 20 μl of SeraMag beads was added to a 1.5 ml microcentrifuge tube containing 1 ml of Enhancing Fluid (2 M NaCl, 10 mM Tris-HCl, 1 mM EDTA, pH 7.5). The SeraMag bead mix was vortexed for 5 seconds, and the tube placed in a Dynal MPC-S magnet, pelleting the paramagnetic beads against the side of the microcentrifuge tube. The supernatant was carefully removed and discarded without disturbing the SeraMag beads, the tube removed from the magnet, and 100 μl of enhancing fluid was added. The tube was vortexed for 3 seconds to resuspend the beads, and the tube stored on ice until needed.

Upon completion of the annealing program, 100 μl of Annealing Buffer was added to the PCR tube containing the DNA capture beads and enrichment primer, the tube vortexed for 5 seconds, and the contents transferred to a fresh 1.5 ml microcentrifuge tube. The PCR tube in which the enrichment primer was annealed to the capture beads was washed once with 200 μl of annealing buffer, and the wash solution added to the 1.5 ml tube. The beads were washed three times with 1 ml of annealing buffer, vortexed for 2 seconds, pelleted as before, and the supernatant carefully removed. After the third wash, the beads were washed twice with 1 ml of ice cold enhancing fluid, vortexed, pelleted, and the supernatant removed as before. The beads were then resuspended in 150 μl ice cold enhancing fluid and the bead solution added to the washed SeraMag beads.

The bead mixture was vortexed for 3 seconds and incubated at room temperature for 3 minutes on a LabQuake tube roller, while the streptavidin-coated SeraMag beads bound to the biotinylated enrichment primers annealed to immobilised templates on the DNA capture beads. The beads were then centrifuged at 2,000 rpm for 3 minutes, after which the beads were gently ‘flicked’ until the beads were resuspended. The resuspended beads were then placed on ice for 5 minutes. Following the incubation on ice, cold Enhancing Fluid was added to the beads to a final volume of 1.5 ml. The tube inserted into a Dynal MPC-S magnet, and the beads were left undisturbed for 120 seconds to allow the beads to pellet against the magnet, after which the supernatant (containing excess SeraMag and null DNA capture beads) was carefully removed and discarded.

The tube was removed from the MPC-S magnet, 1 ml of cold enhancing fluid added to the beads, and the beads resuspended with gentle flicking. It is preferred not to vortex the beads, as vortexing may break the link between the SeraMag and DNA capture beads. The beads were returned to the magnet, and the supernatant removed. This wash was repeated three additional times to ensure removal of all null capture beads.

To remove the annealed enrichment primers and SeraMag beads from the DNA capture beads, the beads were resuspended in 1 ml of melting solution, vortexed for 5 seconds, and pelleted with the magnet. The supernatant, containing the enriched beads, was transferred to a separate 1.5 ml microcentrifuge tube, the beads pelleted and the supernatant discarded. The enriched beads were then resuspended in 1× Annealing Buffer with 0.1% Tween-20. The beads were pelleted on the MPC again, and the supernatant transferred to a fresh 1.5 ml tube, ensuring maximal removal of remaining SeraMag beads. The beads were then centrifuged, after which the supernatant was removed, and the beads washed 3 times with 1 ml of 1× Annealing Buffer. After the third wash, 800 μl of the supernatant was removed, and the remaining beads and solution transferred to a 0.2 ml PCR tube. The average yield for the enrichment process was 30% of the original beads added to the emulsion, or approximately 450,000 enriched beads per emulsified reaction. As a 60× 60 mm² slide requires 900,000 enriched beads, two 1,500,000 bead emulsions were processed as described above.

### Sequencing Primer Annealing

The enriched beads were centrifuged at 2,000 rpm for 3 minutes and the supernatant decanted, after which 15 μl of annealing buffer and 3 μl of 100 mM 454 Seq Forward primer (5′-CCATCTGTTCCCTCCCTGTC-3′; SEQ ID NO: 29; IDT Technologies), were added. The tube was then vortexed for 5 seconds, and placed in an MJ thermocycler for the following 4 stage annealing program: 5 minutes at 65° C., decrease by 0.1° C./sec to 50° C., 1 minute at 50° C., decrease by 0.1° C./sec to 40° C., hold at 40° C. for 1 minute, decrease by 0.1° C./sec to 15° C., hold at 15° C.

Upon completion of the annealing program, the beads were removed from the thermo-cycler and pelleted by centrifugation for 10 seconds, rotating the tube 180°, and spun for an additional 10 seconds. The supernatant was discarded, and 200 μl of annealing buffer was added. The beads were resuspended with a 5 second vortex, and the beads pelleted as before. The supernatant was removed, and the beads resuspended in 100 μl annealing buffer, at which point the beads were quantitated with a Multisizer 3 Coulter Counter. Beads were stored at 4° C. and were stable for at least one week.

Incubation of DNA Beads with Bst DNA Polymerase, Large Fragment and SSB Protein

Bead wash buffer (100 ml) was prepared by the addition of apyrase (Biotage, Uppsala Sweden; final activity 8.5 u/l) to 1× assay buffer containing 0.1% BSA. The fibre-optic slide was removed from picopure water and incubated in bead wash buffer. 900,000 of the previously prepared DNA beads were centrifuged and the supernatant was carefully removed. The beads were then incubated in 1,290 μl of bead wash buffer containing 0.4 mg/ml polyvinyl pyrrolidone (MW 360,000), 1 mM DTT, 175 μg of E. coli single strand binding protein (SSB; United States Biochemicals Cleveland, Ohio) and 7,000 units of Bst DNA polymerase, Large Fragment (New England Biolabs). The beads were incubated at room temperature on a rotator for 30 minutes.

### Preparation of Enzyme Beads and Microparticle Fillers

UltraGlow Luciferase (Promega Madison Wis.) and Bst ATP sulfurylase were prepared in-house as biotin carboxyl carrier protein (BCCP) fusions. The 87-amino acid BCCP region contains a lysine residue to which a biotin is covalently linked during the in vivo expression of the fusion proteins in E. coli. The biotinylated luciferase (1.2 mg) and sulfurylase (0.4 mg) were premixed and bound at 4° C. to 2.0 ml of Dynal M280 paramagnetic beads (10 mg/ml, Dynal SA) according to the manufacturer's instructions.

The enzyme bound beads were washed 3 times in 2,000 μl of bead wash buffer and resuspended in 2,000 μl of bead wash buffer.

Seradyn microparticles (Powerbind SA, 0.8 μm, 10 mg/ml; Seradyn Inc, Indianapolis, Ind.) were prepared as follows: 1,050 μl of the stock were washed with 1,000 μl of 1× assay buffer containing 0.1% BSA. The microparticles were centrifuged at 9,300 g for 10 minutes and the supernatant removed. The wash was repeated two more times and the microparticles were resuspended in 1,050 μl of 1× assay buffer containing 0.1% BSA. The beads and microparticles were stored on ice until use.

### Bead Deposition

The Dynal enzyme beads and Seradyn microparticles were vortexed for one minute and 1,000 μl of each were mixed in a fresh microcentrifuge tube, vortexed briefly and stored on ice. The enzyme/Seradyn beads (1,920 μl) were mixed with the DNA beads (1,300 μl) and the final volume was adjusted to 3,460 μl with bead wash buffer. Beads were deposited in ordered layers. The fibre-optic slide was removed from the bead wash buffer and ‘Layer 1’, a mix of DNA and enzyme/Seradyn beads, was deposited. After centrifuging, Layer 1 supernatant was aspirated off the fibre-optic slide and ‘Layer 2’, Dynal enzyme beads was deposited. This section describes in detail how the different layers were centrifuged.

Layer 1: a gasket that creates two 30× 60 mm² active areas over the surface of a 60× 60 mm² fibre-optic slide was carefully fitted to the assigned stainless steel dowels on the jig top. The fibre-optic slide was placed in the jig with the smooth non-etched side of the slide facing down and the jig top/gasket was fitted onto the etched side of the slide. The jig top was then properly secured with the screws provided, by tightening opposite ends such that they were finger tight. The DNA-enzyme bead mixture was loaded on the fibre-optic slide through two inlet ports provided on the jig top. Extreme care was taken to minimise bubbles during loading of the bead mixture. Each deposition was completed with one gentle continuous thrust of the pipette plunger. The entire assembly was centrifuged at 2,800 rpm in a Beckman Coulter Allegra 6 centrifuge with GH 3.8-A rotor for 10 minutes. After centrifugation the supernatant was removed with a pipette.

Layer 2: Dynal enzyme beads (920 μl) were mixed with 2,760 μl of bead wash buffer and 3,400 μl of enzyme-bead suspension was loaded on the fibre-optic slide as described previously. The slide assembly was centrifuged at 2,800 rpm for 10 min and the supernatant decanted. The fibre-optic slide was removed from the jig and stored in bead wash buffer until ready to be loaded on the instrument.

### Sequencing on the 454 Instrument

All flow reagents were prepared in 1× assay buffer with 0.4 mg/ml polyvinyl pyrrolidone (MW 360,000), 1 mM DTT and 0.1% Tween 20. Substrate (300 μM D-luciferin (Regis, Morton Grove, Ill.) and 2.5 μM adenosine phophosulfate (Sigma)) was prepared in 1× assay buffer with 0.4 mg/ml polyvinyl pyrrolidone (MW 360,000), 1 mM DTT and 0.1% Tween 20. Apyrase wash is prepared by the addition of apyrase to a final activity of 8.5 units per litre in 1× assay buffer with 0.4 mg/ml polyvinyl pyrrolidone (MW 360,000), 1 mM DTT and 0.1% Tween 20. Deoxynucleotides dCTP, dGTP and dTTP (GE Biosciences, Buckinghamshire, United Kingdom) were prepared to a final concentration of 6.5 μM, α-thio deoxyadenosine triphosphate (dATPαS, Biolog, Hayward, Calif.) and sodium pyrophosphate (Sigma) were prepared to a final concentration of 50 μM and 0.1 μM, respectively, in the substrate buffer.

The 454 sequencing instrument consists of three major assemblies: a fluidics subsystem, a fibre-optic slide cartridge/flow chamber, and an imaging subsystem. Reagent inlet lines, a multi-valve manifold, and a peristaltic pump form part of the fluidics subsystem. The individual reagents are connected to the appropriate reagent inlet lines, which allows for reagent delivery into the flow chamber, one reagent at a time, at a pre-programmed flow rate and duration. The fibre-optic slide cartridge/flow chamber has a 300 μm space between the slide's etched side and the flow chamber ceiling. The flow chamber also included means for temperature control of the reagents and fibre-optic slide, as well as a light-tight housing. The polished (non-etched) side of the slide was placed directly in contact with the imaging system.

The cyclical delivery of sequencing reagents into the fibre-optic slide wells and washing of the sequencing reaction by-products from the wells was achieved by a pre-programmed operation of the fluidics system. The program was written in the form of an Interface Control Language (ICL) script, specifying the reagent name (Wash, dATPαS, dCTP, dGTP, dTTP, and PPi standard), flow rate and duration of each script step. Flow rate was set at 4 ml/min for all reagents and the linear velocity within the flow chamber was approximately 1 cm/s. The flow order of the sequencing reagents were organised into kernels where the first kernel consisted of a PPi flow (21 seconds), followed by 14 seconds of substrate flow, 28 seconds of apyrase wash and 21 seconds of substrate flow. The first PPi flow was followed by 21 cycles of dNTP flows (dC-substrate-apyrase wash-substrate, dA-substrate-apyrase wash-substrate, dG-substrate-apyrase wash-substrate, dT-substrate-apyrase wash-substrate) where each dNTP round flow was composed of 4 individual kernels—one for each nucleotide. Each kernel is 84 seconds long (dNTP-21 seconds, substrate flow 14 seconds, apyrase wash-28 seconds, substrate flow-21 seconds); an image is captured after 21 seconds and after 63 seconds. After 21 cycles of dNTP flow, a PPi kernel is introduced, and then followed by another 21 cycles of dNTP flow. The end of the sequencing run is followed by a third PPi kernel. During the run, all reagents were kept at room temperature. The temperature of the flow chamber and flow chamber inlet tubing is controlled at 30° C. and all reagents entering the flow chamber are pre-heated to 30° C.

### In Vitro Transcription/Translation—CIS Display of Peptide Library

An ITT mixture was prepared as described in Example 1 and passed onto the flow cell. The cell was incubated for 1 hour at 25° C. or 30° C. before being washed with PBST and then with PBS. This enabled the peptide-RepA fusions to be expressed and bind to their own DNA template. The beads were blocked with Block Buffer and incubated for 20 min. at room temperature. The beads were then washed with PBST and then with PBS. A solution of DYKDDDDK Tag Alexa Fluor® 647 conjugated antibody (NEB; 1:500 or 1:1000 in PBS containing 2% BSA) was then added and incubated at room temperature for 1 hour. This was again washed with PBST and then with PBS.

The fluorescent signal corresponding to binding of the antibody to the FLAG epitope present in library peptides immobilised on the flow cell was measured by laser excitation at 630 nm or 650 nm with monitoring of the emission at 668 nm.

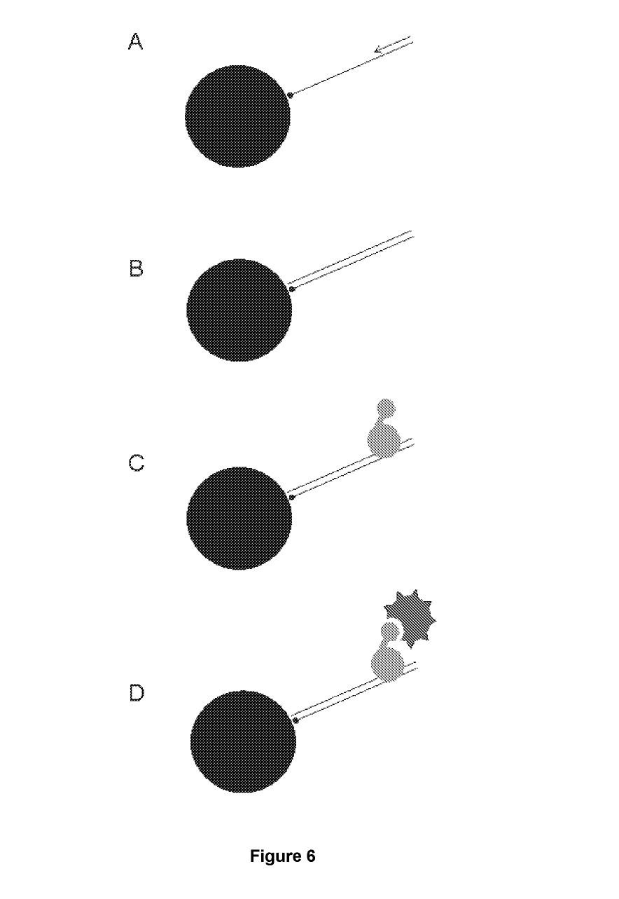

This example is shown schematically in FIG. 6. As described previously, the in situ sequencing and screening method of the invention is suitable for use with any second generation or next-generation sequencing procedure, providing the sequencing platform is compatible with immobilised nucleic acid molecules. Hence, the procedure with the 454 sequencing platform described in this Example can be replaced by any other appropriate sequencing platform, for example, as described below. Alternatively, sequencing can be performed in situ after peptide library expression.

The P2A may alternatively be used in the processes described in the Examples herein, with the A protein from P2 phage (P2A) replacing the RepA protein C/S and ori. By way of example, the template tacP2AHA (SEQ ID NO: 48) is made and amplified with primers LAMPB (SEQ ID NO: 49) and P2AAmpf (SEQ ID NO: 51) using the methods previously described (Reiersen et al., (2005), NAR, 33, e10). The amplified product is then purified using Qiagen columns and used as a template for further amplification with LAMPB and LinkP2Afor (SEQ ID NO: 50). Following purification, the product, Link-P2A (SEQ ID NO: 52), was then amplified with primers flaglib-p2afor (SEQ ID NO: 53) and LAMPB to form template flaglib-P2A (SEQ ID NO: 54). flaglib-P2A was purified and further amplified with primers 131-mer and LAMPB to append the tac promoter and form the template tacflaglib-P2A (SEQ ID NO: 55). Further PCR amplification, after purification, with Adapter A and Adapter C (SEQ ID NO: 56) was performed to produce the product tac-flaglib-P2A-454-adapted (SEQ ID NO: 57) which can be used in Roche 454 sequencing. Similarly modified constructs of P2A may be used for other sequencing methods (as described herein with respect to RepA templates), and for in vitro transcription and translation and peptide screening.

### Ion Torrent Sequencing

As an alternative to sequencing on the 454 instrument, Ion Torrent sequencing based on the chemically-sensitive field effect transistor (chemFET) approach may be used, as described, for example, in Rothberg et al., 2011, Nature, 475, 348-352 and supplementary materials, US2010/0282617, and US2011/0287945,

The dimensions and density of the ISFET array and the microfluidics positioned thereon may vary depending on the application.

For sequencing using the ISFET chip, the methods are very similar to those for the Roche 454 sequencing method. The template is prepared using a forward primer (Primer A-key; SEQ ID NO: 32), and a reverse primer (Primer P1-key; SEQ ID NO: 33) to produce tac-flaglib-repA-CIS-ori-ionadapt (SEQ ID NO: 41). The template is amplified through emulsion PCR captured though annealing of the Primer P1-key sequence to the capture beads, 5.91 μm diameter streptavidin-coated beads (Bangs Laboratories, Inc. Fishers, Ind.), and sequencing from the A-key primer or Ion Torrent sequencing adapters. These fragments are clonally amplified on the Ion Sphere™ particles by emulsion PCR. The Ion Sphere™ particles with the amplified template are then applied to the Ion Torrent chip and the chip is placed on the Ion PGM™. The sequencing run is set up on the Ion PGM™. Sequencing results are provided in standard file formats. Downstream data analysis can be performed using the DNA-Seq workflow of the Partek® Genomics Suite™.

Briefly, the reagents are flowed in a sequential manner across the chip surface, extending a single DNA base(s) at a time. The dNTPs are flowed sequentially, beginning with dTTP, then dATP, dCTP, and dGTP. Washes between nucleotide additions were conducted with 6.4 mM MgCl₂, 13 mM NaCl, 0.1% Triton X-100 at pH 7.5. The flow regime also ensures that the vast majority of nucleotide solution is washed away between applications. This involves rinsing the chip with buffer solution and apyrase solution following every nucleotide flow. The ISFET chip is activated for sensing chemical products of the DNA extension during nucleotide flow according to manufacturer's instructions, Ion Torrent user guide (Life Technologies) and Margulies et al., (2005), Nature, 437(15), 376-380 and accompanying supplemental materials.

### In Vitro Transcription/Translation—CIS Display of Peptide Library

Following sequencing through the library region, all 4 dNTPS are delivered together to completely fill-in the remainder of the RepA sequence thereby generating a double stranded DNA template using Bst polymerase as previously described. The fill-in reagents are then flushed from the system in assay buffer and ITT components are delivered according to the previous example, i.e. at a ratio of 40% 2.5× buffer, 20% water, 10% amino acid mix (1 mM) and 30% S30 lysate which has been centrifuged at 16,000 g for 10 min in a microfuge.

The ITT is incubated in the slide for 1 hour at 25° C. or 30° C. and then the flow chamber is flushed with PBST containing 2% BSA and then PBS. A solution of anti-FLAG HRP is then flowed though the chamber, followed by a wash with PBST, and finally a wash with phosphate buffer at pH 6.0. The bound anti-FLAG HRP was detected with o-phenilendiamine in a solution of the phosphate buffer pH 6.0, containing 0.25 mM o-phenilendiamine and of 0.125 mM H₂O₂ (Kergaravat et al., (2012), Talanta, 88, 468-476).

### SOLiD™ Sequencing

Yet another possible system for sequencing the immobilised nucleic acids is the SOLiD™ sequencing system (Applied Biosystem)

### Example 8

### Affinity Measurement

Affinity measurements may be made on any of the sequencing arrays described in the examples above following the formation of the protein-DNA complexes. The affinity measurement can be made either with or without modification to the instrument or platform.

First, we exemplify a procedure for affinity measurements on a planar surface as described above (Examples 6 and 7) for the Illumina platform without modification of the instrument. Following the expression from the tac-flaglib-repA-CIS-ori DNA sequence to form peptide-DNA complexes, peptides bound to the anti-FLAG antibody can be detected. A 2 minute wash with PBST containing 2% BSA was performed followed by a 2 minute PBST wash. Anti-DYKDDDDK Tag Alexa Fluor® 647 conjugated antibody (NEB) diluted 1 in 500 in PBST was added to the array. Alternatively, anti-FLAG Cy5.5 antibody can be used (www.proteinmods.com). Binding was noted by exciting the clusters on the array at 630 nm or 650 nm and reading the emission signal at 668 nm.

As previously described, the optics of the Illumina system are based upon internal reflection illumination of the fluorophores which excites only fluorophores situated <100 nm from the flow cell surface, which allows the system to discriminate between fluorophores attached to the surface and those free in solution. The length of the DNA-protein complex is will within this detection range (typically being less than 5 nm), and a wash step may not be necessary after addition of the DYKDDDDK Tag Alexa Fluor® 647. Having measured the signal without a wash step, if the background signal is found to be too high a wash step may be included (e.g. a suitable wash may comprise of a gentle flow of PBST over the array followed by PBS). The cluster size and the background fluorescence signals were normalised and the background fluorescence was subtracted from the averaged normalised signal for the FLAG epitope expressing clusters. The intensity of the signal above background versus the concentration of the anti-DYKDDDDK Tag Alexa Fluor® 647 antibody can be plotted and fitted to a Hill's equation in order to determine the dissociation constant (Kd).

### Example 9

### Multiplex Selectivity

The selectivity of the binding to the immobilised peptide can be tested by incubating the slide, either simultaneously or sequentially, with both anti-DYKDDDDK Tag Alexa Fluor® 647 antibody and other proteins such as anti-V5 antibody conjugated with Alexa Fluor® 488 which has different excitation and emission properties to the anti-DYKDDDDK Tag Alexa Fluor® 488 antibody. Those peptides that are cross reactive will have fluorescence at both 519 nm and 668 nm when excited at 488 nm and 630 nm or 650 nm respectively. The fluorescence will be seen from the cluster formed from a single DNA species. Those peptides that are specific to the FLAG paratope of the antibody will only emit fluorescence near 668 nm.

### Example 10

### Competition Experiment

The array can be used to assess the affinity of a molecule for a particular binding site displayed on the surface of the array attached to its coding nucleic acid. In this example, the bound anti-DYKDDDDK Tag Alexa Fluor® 647 antibody bound to the surface of the array is chased with a FLAG peptide of sequence DYKDDDDK at a concentration of 1 to 50 nM. Those sequences in the array that are weakly bound by the antibody will be eluted by competition with the solution phase FLAG peptide.

### Example 11

### Library Selection on a Planar Surface

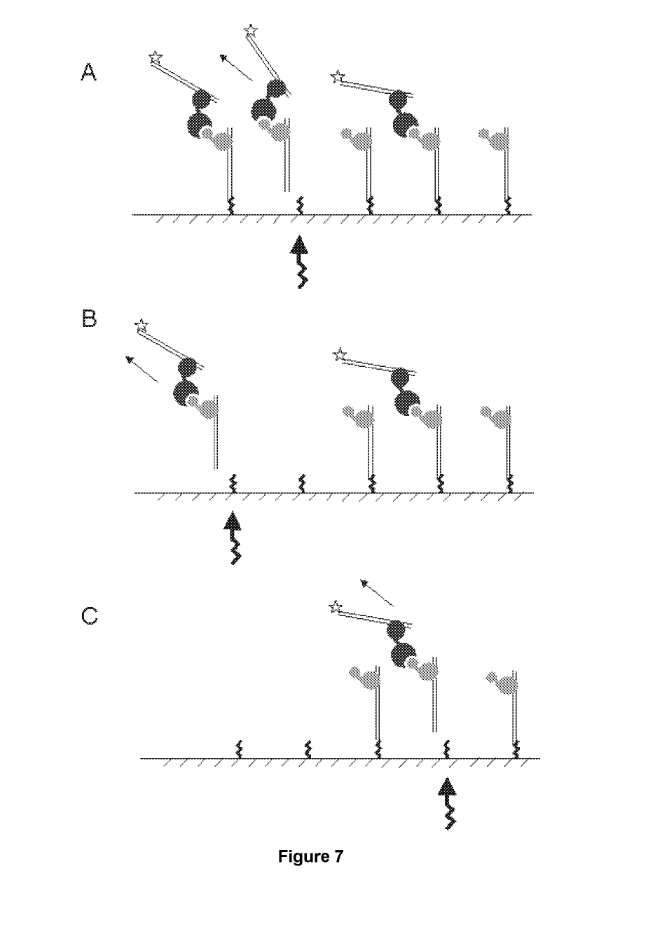

The array can be used to multiplex selections to different targets, as illustrated schematically in FIG. 7. A 6-mer peptide library was made by amplifying the 1steprepA template as described in Example 4 with a degenerate oligo 6mer-libfor (SEQ ID NO: 34) used in place of flag-libfor. The subsequent PCR with primers 131-mer and 85-Orirev was identical to that for flag-libfor, except that the resulting DNA product contained 6×NNS codons and was called tac-6merlib-repA-CIS-ori (“Library 1”; SEQ ID NO: 42) which was subsequently amplified by primers D and E as described in the example above to create tac-6merlib-repA-CIS-ori-illumadapt (SEQ ID NO: 43).

A second library was made based upon a VWV domain sequence as described in our co-pending patent application (PCT/GB2011/051500). This library was made using the same procedures as described for 6merlib and flaglib but using the Pinlibfor primer (SEQ ID NO: 35) from PCT/GB2011/051500 to create tac-pinlib-repA-CIS-ori (SEQ ID NO: 45).

The Illumina flow cell was treated as described above (Example 4); however, the surface was modified with an oligo containing a photocleavable linker, created by synthesis of the oligonucleotide with a photocleavable phosphoramidite spacer (such as PC Spacer Phosphoramidite distributed by Glen Research, Stirling, Va.; or as described by Li et al., 2003, PNAS 100, 414-419). The oligonucleotide D2 5′-PS-PC-TTTTTTTTTTCAAGCAGAAGACGGCATACGAGoxoAT-3′ (SEQ ID NO: 36), in which PC represents a photocleavable spacer, PS is a phosphorothioate oligonucleotide, was prepared by Integrated DNA Technologies, (Leuven, Belgium) and was used in place of oligo D′ on the surface of the chip.

The DNA templates from Library 1 (tac-6merlib-repA-CIS-ori-illumadapt) were then arrayed on the array surface, and this was followed by bridge amplification and sequencing as described above (Example 4).

In vitro transcription/translation (ITT) was performed as previously described (Example 5) to produce proteins fused to RepA that were displayed on the surface of the array as protein-DNA complexes. The array was blocked by passing a solution of Block Buffer over the surface of the chip.

Another library (“Library 2”) tac-pinlib-repA-CIS-ori was amplified without the Illumina adapter sequences (to prevent immobilisation on the surface of the array). This template was labelled with Alexa Fluor® 647 at the 3′ end of on using an Orirev primer labelled with the Alexa Fluor® 647 dye (OrirevAlex647, SEQ ID NO: 37). A 100 μl in vitro transcription and translation reaction was performed in a tube according to the protocols described above, blocked with 900 μl of Block Buffer, and the ITT protein mixture was then passed over the array of Library 1 proteins immobilised on the slide.

Binding of Library 2 members to Library 1 members was monitored by exposing the bridge-amplified clusters to light at 630 nm or 650 nm and recording the emission at 668 nm. Those clusters where there was a signal at 668 nm were then exposed to light at 320-340 nm from a laser beam focussed to a point precisely matching the positive cluster (this point is anticipated to be approximately between 500 nm to 2 μm in diameter) for between 5 seconds and 30 minutes in order to release the DNA from the surface and release the attached protein-protein complexes. The slide was then washed with buffer and the wash was collected by precisely switching the flow to a collection device such as a collection plate or tube via tubing (such as polyetheretherketone tubing) so that the collected DNA could be PCR amplified using primers specific for Library 2, e.g. 5′ phosphorylated primers Pinlibfor (SEQ ID NO: 46) and Pinlibrev (SEQ ID NO: 47). Following this, the PCR products were column purified and sequenced either using next generation methods or cloned into pUC18 plasmid, previously digested with Smal and treated with alkaline phosphatase (pUC18-Smal-AP, Bayou Biolabs, LA), and subsequently purified from colonies using miniprep procedure using Qiaprep Miniprep Kit (Qiagen, Crawley, West Sussex, UK). Finally, PCR products were sequenced using Sanger sequencing.

The flow of wash fluid through the cell may be controlled by monitoring the fluorescent signal associated with the Library 2 complexes being released form the surface and switching the direction of the flow appropriately.

As an alternative, tac-6merlib-repA-CIS-ori (“Library 1”; SEQ ID NO: 42) could be amplified by primers Adapter A (SEQ ID NO: 25) and Adapter B (SEQ ID NO: 26) as described in Example 7 to create tac-6merlib-repA-CIS-ori-454adapt (SEQ ID NO: 44) for sequencing using the 454 instrument as previously described.

### Example 12

### Library Selection on a Planar Surface

The array can be used to multiplex selections to different targets, as illustrated schematically in FIG. 7. In this Example, two 15-mer peptide libraries based on the experiments described in Wang & Pabo (1999) “Dimerization of zinc fingers mediated by peptides evolved in vitro from random sequences”, Proc. Natl. Acad. Sci. USA, 96(17): 9568-73A were designed.

A first 15-mer peptide library was made by amplifying the 1steprepA template as described in Example 4 with a degenerate oligo 15mer-lib1for (SEQ ID NO: 62) used in place of flag-libfor. The subsequent PCR with primers 131-mer and 85-Orirev was identical to that for flag-libfor, except that the resulting DNA product contained 15 degenerate codons and was called tac-15merlib1-repA-CIS-ori (“Library 1”; SEQ ID NO: 64) which was subsequently amplified by primers D and E as described in the example above to create tac-15merlib1-repA-CIS-ori-illumadapt (SEQ ID NO: 65).

A second library was made based upon a second 15-mer peptide sequence. This library was made using the same procedures as described for 15mer-lib1for and flaglib but using the 15mer-lib2for primer (SEQ ID NO: 63) to create tac-15merlib2-repA-CIS-ori (SEQ ID NO: 67).

The Illumina flow cell was treated exactly as described in Example 11 above.

The DNA templates from Library 1 (tac-15merlib1-repA-CIS-ori-illumadapt) were then arrayed on the array surface, and this was followed by bridge amplification and sequencing as described above (Example 4).

In vitro transcription/translation (ITT) was performed as described in Example 11.

Another library (“Library 2”) tac-15merlib2-repA-CIS-ori was amplified without the Illumina adapter sequences (to prevent immobilisation on the surface of the array). This template was labelled with Alexa Fluor® 647 at the 3′ end of on using an Orirev primer labelled with the Alexa Fluor® 647 dye (OrirevAlex647, SEQ ID NO: 37). A 100 μl in vitro transcription and translation reaction was performed in a tube according to the protocols described in Example 11.

Binding of Library 2 members to Library 1 members was monitored as described in Example 11, except that the collected DNA was PCR amplified using primers specific for the 15mer Library 2, e.g. 5′ phosphorylated primers 15merlib2-recoveryfor (SEQ ID NO: 68) and 15merlib2-recoveryrev (SEQ ID NO: 69). Following this, the PCR products were purified and sequenced as described in Example 11.

As an alternative, tac-15merlib1-repA-CIS-ori (“Library 1”; SEQ ID NO: 64) could be amplified by primers Adapter A (SEQ ID NO: 25) and Adapter B (SEQ ID NO: 26) as described in Example 7 to create tac-15merlib1-repA-CIS-ori-454adapt (SEQ ID NO: 66) for sequencing using the 454 instrument as previously described.

### Example 13

### Library Selection on a Bead Surface

As described in Examples 11 and 12 above, multiplex target selections can be performed on a NGS sequencing instrument on a planar surface (e.g. a slide), or may alternatively be performed on beads as the solid surface on which Library 1 members are immobilised.

Accordingly, in this alternative method, Library 1 is immobilised to a bead surface and is sequenced as previously described (Example 7); followed by a fill-in polymerase reaction to reconstitute the double-stranded template molecule. The template is then subjected to an ITT step where the Library 1 proteins are tethered to their own DNA through the DNA binding action of RepA followed by a flow of Block Buffer over the array. Instead of RepA any other suitable cis-binding agent/mechanism may alternatively be used.

Library 2 protein-DNA fusions are then made by ITT and passed over the beads trapped in microwells as described previously (Example 7). The Library 2 members are either not capable of being immobilised to the solid support on which Library 1 members are immobilised, or they are not capable of being immobilised in this way under the conditions used in this step. The wells are then washed with PBST and with PBS, and the fluorescence is determined at 668 nm to identify the beads that have Library 2 members bound/attached thereto. These beads can then be picked from specific sites on the array using a microactuator-controlled micropipette guided by cameras. The recovered beads can then be amplified using PCR so that the DNA templates encoding the binding population for each bead are enriched. PCR products can then be cloned to identify the two (or potentially more) DNA fragments that encode the peptides that were responsible for the recovered binding event.

Alternatively, the beads can be irradiated using a laser device focussed upon the wells identified as containing Library 2 binders. Preferably, the beam of the laser will have a diameter that is less than the diameter of the microwells (which are 44 μm by 55 μm in the Roche array), or as small as 0.5 μm, for between 5 seconds and 30 minutes duration. The DNA-protein complexes are thus released from the bead surface and can be collected from the array, e.g. following a flow of buffer such as PBS over the surface and collecting the wash (eluate) by precisely switching the flow to a collection device such as a collection plate or tube. The collected DNA can then be PCR amplified using primers specific for Library 2 templates. Following amplification of captured templates, the PCR products may be cloned and/or directly sequenced using next generation methods or using standard Sanger sequencing.

Alternatively, it can be envisaged that by immobilising Library 1 on paramagnetic beads, an electromagnetic switch could be used to collect or release the appropriate beads from the wells of the array.

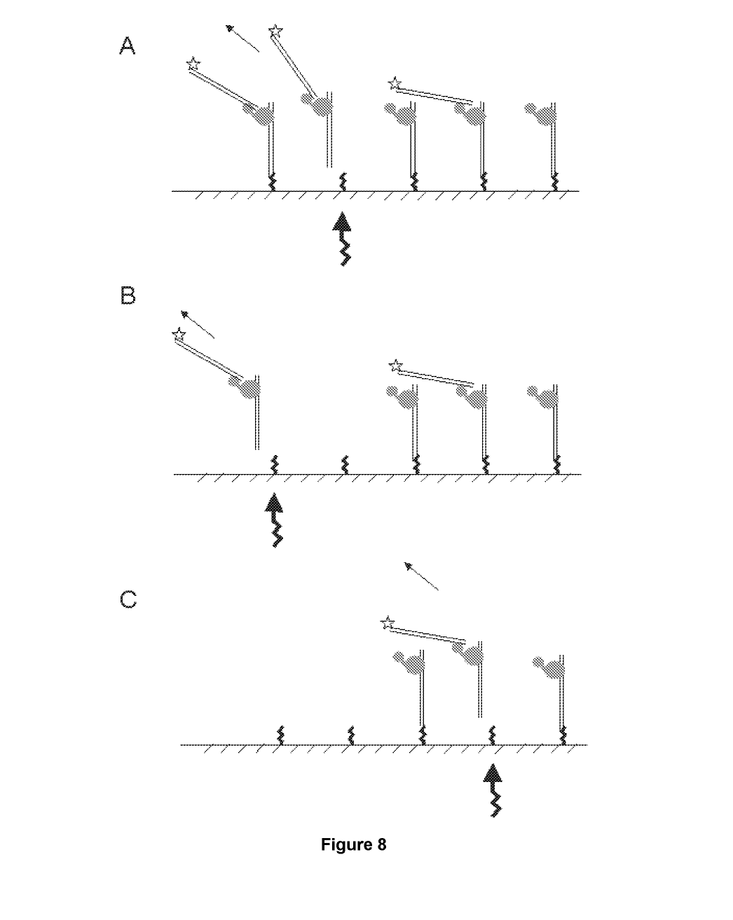

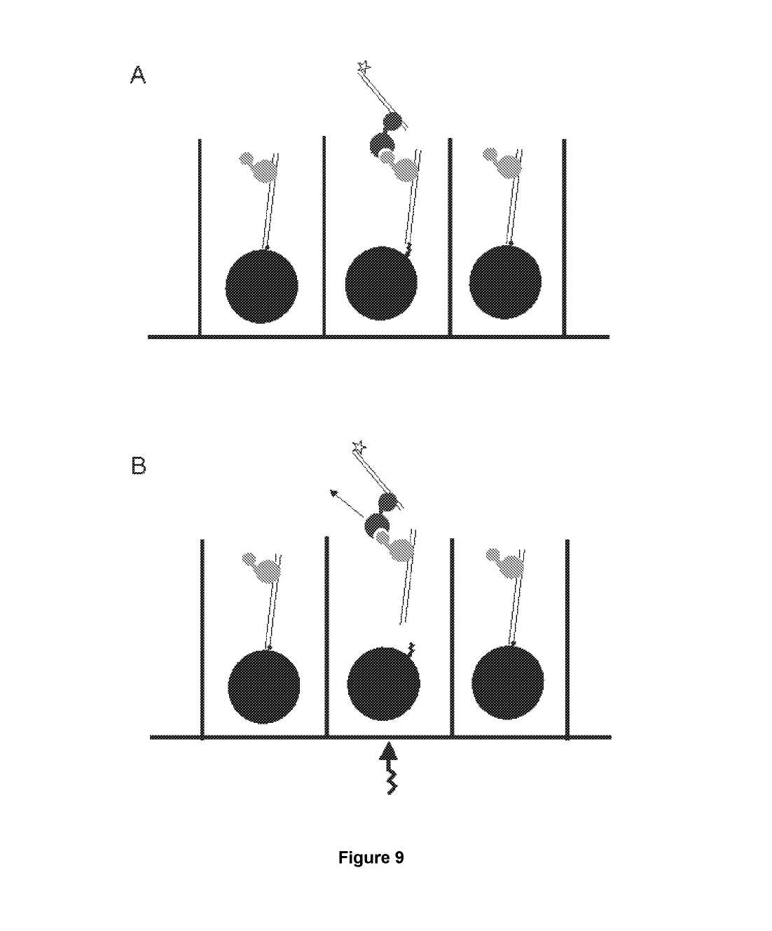

The processes for library selection are shown diagrammatically in FIGS. 7, 8 and 9.

### Example 14

### In Vitro Peptide Library Expression, Nucleic Acid Immobilisation, Library Selection

Protein DNA complexes can be made prior to sequencing using CAPs or mRNA display methods. The mRNA templates and peptide nucleic acid fusions can be made using methods described in the literature as reviewed by Douthwaite & Jackson, “Ribosome Display and Related Technologies” Edited by Douthwaite & Jackson, 2012, Methods in Molecular Biology, Volume 805, Springer Press), or as described in WO 2011/0183863 via the action of puromycin, pyrazolopyrimidine, streptavidin-biotin linkage or any other linker. It is also envisaged that macrocycles may also be tethered to the DNA for use in arrays. Such methods of attachment are described in patent application WO 02/074929 and peptide fusion methods outlined below are described in further detail in WO 2011/0183863.

For example, an RNA template is made using a MEGAscript Kit (Ambion, Foster City, Calif.) to transcribe PCR amplified DNA into RNA. The RNA is then purified by adding an equal volume of 10 M LiCI, mixing, and freezing at −20° C. for 1 hour. The sample is then centrifuged at 13,500 g in a microfuge for 20 minutes and the supernatant discarded. The pellet is resuspended in 1.5 M sodium acetate followed by ethanol precipitation with 2.5 volumes of chilled ethanol. Following incubation at −20° C., the sample is centrifuged at 13,500 g in a microfuge for 10 minutes and washed with 1 ml 70% ethanol at 4° C. The sample iss centrifuged again and the washing process repeated at least once more. The pellet is dried in air and resuspended in water and the RNA concentration measured using Qubit (Life Technologies, Paisley, U.K.) or Nanodrop (Termo Scientific, Wilmington, Del.), or an equivalent suitable system.

A DNA oligonucleotide (Linker) that has 19 complementary bases to the 3′ end of the PCR product (upstream of the poly A tail) and 5′-(Psoralen C6) C7-NH₂-EZ-Biotin (EZ-link TFP-spacer-biotin) linked to the DNA bases (supplied by Trilink Bio Technologies Inc., San Diego, Calif.) is mixed in a 1.5-1.1 molar excess to the RNA (100-600 pmol) in 25 mM Tris pH 7 and 100 mM NaCl, and heated at 85° C. for 30 seconds; then cooled to 4° C. at a rate of less than 1° C. per second in order to anneal the DNA Linker to the RNA. 1 mM DTT is added to the mixture and the mix is then irradiated with a UV lamp (UVP, Upland Calif.) at 365 nm for 5-10 minutes at room temperature in order to crosslink the DNA oligonucleotide to the mRNA. Streptavidin is then loaded on the biotinylated hybrid using 1.5-2 molar excess of mRNA over streptavidin in 20 mM HEPES, pH 7.4, 100 mM NaCl. 1 μl RNAsin (Promega, Madison, Wis.) can then be added and incubated at 48° C. for 1 hour. A further linker that carries 5′-biotin-(8× spacer 18)-puromycin is added to the DNA-RNA-streptavidin complex at a molar ratio of 1:1 in order to link puromycin to the RNA/DNA template. Purification is performed through precipitation with LiCI as described above, or using oligo-dT cellulose (Sigma, Poole. UK).

Translation of the mRNA is performed using 40 pmol RNA in water per 100 μl translation reaction using Retic lysate IVT Kit (Life Technologies) for 1 hour at 30° C. Following translation the protein DNA fusions are formed by addition of 500 mM KCl and 50 mM MgCl₂ final concentration and incubating for 1 hour at room temperature, followed by freezing. The ribosomes are dissociated from the templates by the addition of 50 mM EDTA, pH 8. The fusions are purified by oligo dT cellulose by addition of an equivalent volume of binding buffer (200 mM Tris, pH 8, 2 M NaCl, 20 mM EDTA, 0.1% Triton X-100) incubated at 4° C. for 30-60 minutes, followed by washing by adding the mixture to a spin column (Biorad), centrifuging in a microfuge at 1500 rpm, and resuspending the pellet in 100 mM Tris, pH 8, 1 M NaCl, 0.1% Triton X-100. Following up to 8 washes the fusions are equilibrated in 1× First strand buffer (Superscript II Kit, Life Technologies, Paisley, UK), 50 mM Tris-HCl (pH 8.3), 75 mM KCl, 3 mM MgCl₂.

Reverse transcription is then performed using Superscript II according to manufacturers' instructions for 60-75 min at 37° C. Enzyme concentrations and dNTPs may be increased to improve yield. The RNA strand is then digested with RNAseH (2U/100 μl mixture) for 1 hour at 37° C., and the single-stranded DNA fusions are eluted by spinning the oligo dT column at 2000 rpm and then washing with 5 mM Tris, pH 7. The free biotin streptavidin sites are blocked by adding 0.5 molar equivalent of free biotin to the fusions in order to maintain a high Tm for the complex.

DNA-peptide complexes are then used to anneal to a planar or bead surface, for example via complementary sequences to or C′ and D′ primers as described in Example 4 above.

The DNA-peptide complexes are then assayed for ligand binding as described for Examples 8 to 12 followed by sequencing, as described in Examples 4 to 7.

TABLE 1
Primer, template, peptide and expression construct
sequences (U represents 2-deoxyuridine; Goxo
represents 8-oxoguanine; * represents a phos-
phorothioate bond; Bio represents biotin; T^{bio}
represents an internal Biotin dT); C₁₂H₂₆O₇
represents hexa-ethylene glycol (HEG); C₆H₁₄O₄
is Tri-ethylene glycol (TEG)

tac-CK-repA-CIS-ori sequence (SEQ ID NO: 1)
CGGCGGTTAGAACGCGGCTACAATTAATACATAACCCCATCCCCCTGTTGACAATTAATCATcGG
CTCGTATAATGTGTGGAATTGTGAGCGGATAACAATTTCACACAGGAAACAGGATCTACCATGGC
CGGATCTACCATGGCCCAGATACGCGCCACTGTGGCTGCACCATCTGTCTTCATCTTCCCGCCAT
CTGATGAGCAGTTGAAATCTGGAACTGCCTCTGTTGTGTGCCTGCTGAATAACTTCTATCCCAGA
GAGGCCAAAGTACAGTGGAAGGTGGATAACGCCCTCCAATCGGGTAACTCCCAGGAGAGTGTCAC
AGAGCAGGACAGCAAGGACAGCACCTACAGCCTCAGCAGCACCCTGACGCTGAGCAAAGCAGACT
ACGAGAAACACAAAGTCTACGCCTGCGAAGTCACCCATCAGGGCCTGAGCTCGCCCGTCACAAAG
AGCTTCAACAGGGGAGGCAGCGGTTCTAGTCTAGCGGCCCCAACTGATCTTCACCAAACGTATTA
CCGCCAGGTAAAGAACCCGAATCCGGTGTTCACTCCCCGTGAAGGTGCCGGAACGCTGAAGTTCT
GCGAAAAACTGATGGAAAAGGCGGTGGGCTTCACCTCCCGTTTTGATTTCGCCATTCATGTGGCG
CATGCCCGTTCCCGTGGTCTGCGTCGGCGCATGCCACCGGTGCTGCGTCGACGGGCTATTGATGC
GCTGCTGCAGGGGCTGTGTTTCCACTATGACCCGCTGGCCAACCGCGTCCAGTGTTCCATCACCA
CACTGGCCATTGAGTGCGGACTGGCGACAGAGTCCGGTGCAGGAAAACTCTCCATCACCCGTGCC
ACCCGTGCCCTGACGTTCCTGTCAGAGCTGGGACTGATTACCTACCAGACGGAATATGACCCGCT
TATCGGGTGCTACATTCCGACCGACATCACGTTCACACTGGCTCTGTTTGCTGCCCTTGATGTGT
CTGAGGATGCAGTGGCAGCTGCGCGCCGCAGTCGTGTTGAATGGGAAAACAAACAGCGCAAAAAG
CAGGGGCTGGATACCCTGGGTATGGATGAGCTGATAGCGAAAGCCTGGCGTTTTGTGCGTGAGCG
TTTCCGCAGTTACCAGACAGAGCTTAAGTCCCGTGGAATAAAACGTGCCCGTGCGCGTCGTGATG
CGAACAGAGAACGTCAGGATATCGTCACCCTGGTGAAACGGCAGCTGACGCGCGAAATCTCGGAA
GGACGCTTCACTGCTAATGGTGAGGCGGTAAAACGCGAAGTGGAGCGTCGTGTGAAGGAGCGCAT
GATTCTGTCACGTAACCGCAATTACAGCCGGCTGGCCACAGCTTCTCCCTGAAAGTGATCTCCTC
AGAATAATCCGGCCTGCGCCGGAGGCATCCGCACGCCTGAAGCCCGCCGGTGCACAAAAAAACAG
CGTCGCATGCAAAAAACAATCTCATCATCCACCTTCTGGAGCATCCGATTCCCCCTGTTTTTAAT
ACAAAATACGCCTCAGCGACGGGGAATTTTGCTTATCCACATTTAACTGCAAGGGACTTCCCCAT
AAGGTTACAACCGTTCATGTCATAAAGCGCCAGCCGCCAGTCTTACAGGGTGCAATGTATCTTTT
AAACACCTGTTTATATCTCCTTTAAACTACTTAATTACATTCATTTAAAAAGAAAACCTATTCAC
TGCCTGTCCTGTGGACAGACAGATATGCA
S-R1RecFor (SEQ ID NO: 2)
g*a*acgcggctacaattaatacataacc
#514 ThioBioXho85 (SEQ ID NO: 3)
G*G*T^{bio}GATCAGTCAGCTCGAGtgcatatctgtctgtccacagg
tac-CK-repA-CIS-ori-bio (SEQ ID NO: 4)
G*A*ACGCGGCTACAATTAATACATAACCCCATCCCCCTGTTGACAATTAATCATcGGCTCGTAT
AATGTGTGGAATTGTGAGCGGATAACAATTTCACACAGGAAACAGGATCTACCATGGCCGGATCT
ACCATGGCCCAGATACGCGCCACTGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTCACAAAGAGCTTCA
ACAGGGGAGGCAGCGGTTCTAGTCTAGCGGCCCCAACTGATCTTCACCAAACGTATTACCGCCAG
GTAAAGAACCCGAATCCGGTGTTCACTCCCCGTGAAGGTGCCGGAACGCTGAAGTTCTGCGAAAA
ACTGATGGAAAAGGCGGTGGGCTTCACCTCCCGTTTTGATTTCGCCATTCATGTGGCGCATGCCC
GTTCCCGTGGTCTGCGTCGGCGCATGCCACCGGTGCTGCGTCGACGGGCTATTGATGCGCTGCTG
CAGGGGCTGTGTTTCCACTATGACCCGCTGGCCAACCGCGTCCAGTGTTCCATCACCACACTGGC
CATTGAGTGCGGACTGGCGACAGAGTCCGGTGCAGGAAAACTCTCCATCACCCGTGCCACCCGTG
CCCTGACGTTCCTGTCAGAGCTGGGACTGATTACCTACCAGACGGAATATGACCCGCTTATCGGG
TGCTACATTCCGACCGACATCACGTTCACACTGGCTCTGTTTGCTGCCCTTGATGTGTCTGAGGA
TGCAGTGGCAGCTGCGCGCCGCAGTCGTGTTGAATGGGAAAACAAACAGCGCAAAAAGCAGGGGC
TGGATACCCTGGGTATGGATGAGCTGATAGCGAAAGCCTGGCGTTTTGTGCGTGAGCGTTTCCGC
AGTTACCAGACAGAGCTTAAGTCCCGTGGAATAAAACGTGCCCGTGCGCGTCGTGATGCGAACAG
AGAACGTCAGGATATCGTCACCCTGGTGAAACGGCAGCTGACGCGCGAAATCTCGGAAGGACGCT
TCACTGCTAATGGTGAGGCGGTAAAACGCGAAGTGGAGCGTCGTGTGAAGGAGCGCATGATTCTG
TCACGTAACCGCAATTACAGCCGGCTGGCCACAGCTTCTCCCTGAAAGTGATCTCCTCAGAATAA
TCCGGCCTGCGCCGGAGGCATCCGCACGCCTGAAGCCCGCCGGTGCACAAAAAAACAGCGTCGCA
TGCAAAAAACAATCTCATCATCCACCTTCTGGAGCATCCGATTCCCCCTGTTTTTAATACAAAAT
ACGCCTCAGCGACGGGGAATTTTGCTTATCCACATTTAACTGCAAGGGACTTCCCCATAAGGTTA
CAACCGTTCATGTCATAAAGCGCCAGCCGCCAGTCTTACAGGGTGCAATGTATCTTTTAAACACC
TGTTTATATCTCCTTTAAACTACTTAATTACATTCATTTAAAAAGAAAACCTATTCACTGCCTGT
CCTGTGGACAGACAGATATGCACTCGAGCTGACTGATCbioA*C*C
tac-V5-repA-CIS-ori (SEQ ID NO: 5)
CCCCATCCCCCTGTTGACAATTAATCATcGGCTCGTATAATGTGTGGAATTGTGAGCGGATAACA
ATTTCACACAGGAAACAGGATCTACCATGGCCGCAGGAAAACCTATCCCAAACCCTCTCCTAGGA
CTGGATTCAACGGGCAGCGGTTCTAGTCTAGCGGCCCCAACTGATCTTCACCAAACGTATTACCG
CCAGGTAAAGAACCCGAATCCGGTGTTCACTCCCCGTGAAGGTGCCGGAACGCTGAAGTTCTGCG
AAAAACTGATGGAAAAGGCGGTGGGCTTCACCTCCCGTTTTGATTTCGCCATTCATGTGGCGCAT
GCCCGTTCCCGTGGTCTGCGTCGGCGCATGCCACCGGTGCTGCGTCGACGGGCTATTGATGCGCT
GCTGCAGGGGCTGTGTTTCCACTATGACCCGCTGGCCAACCGCGTCCAGTGTTCCATCACCACAC
TGGCCATTGAGTGCGGACTGGCGACAGAGTCCGGTGCAGGAAAACTCTCCATCACCCGTGCCACC
CGTGCCCTGACGTTCCTGTCAGAGCTGGGACTGATTACCTACCAGACGGAATATGACCCGCTTAT
CGGGTGCTACATTCCGACCGACATCACGTTCACACTGGCTCTGTTTGCTGCCCTTGATGTGTCTG
AGGATGCAGTGGCAGCTGCGCGCCGCAGTCGTGTTGAATGGGAAAACAAACAGCGCAAAAAGCAG
GGGCTGGATACCCTGGGTATGGATGAGCTGATAGCGAAAGCCTGGCGTTTTGTGCGTGAGCGTTT
CCGCAGTTACCAGACAGAGCTTAAGTCCCGTGGAATAAAACGTGCCCGTGCGCGTCGTGATGCGA
ACAGAGAACGTCAGGATATCGTCACCCTGGTGAAACGGCAGCTGACGCGCGAAATCTCGGAAGGA
CGCTTCACTGCTAATGGTGAGGCGGTAAAACGCGAAGTGGAGCGTCGTGTGAAGGAGCGCATGAT
TCTGTCACGTAACCGCAATTACAGCCGGCTGGCCACAGCTTCTCCCTGAAAGTGATCTCCTCAGA
ATAATCCGGCCTGCGCCGGAGGCATCCGCACGCCTGAAGCCCGCCGGTGCACAAAAAAACAGCGT
CGCATGCAAAAAACAATCTCATCATCCACCTTCTGGAGCATCCGATTCCCCCTGTTTTTAATACA
AAATACGCCTCAGCGACGGGGAATTTTGCTTATCCACATTTAACTGCAAGGGACTTCCCCATAAG
GTTACAACCGTTCATGTCATAAAGCGCCAGCCGCCAGTCTTACAGGGTGCAATGTATCTTTTAAA
CACCTGTTTATATCTCCTTTAAACTACTTAATTACATTCATTTAAAAAGAAAACCTATTCACTGC
CTGTCCTGTGGACAGACAGATATGCA
tac-V5-repA-CIS-ori-bio (SEQ ID NO: 6)
CCCCATCCCCCTGTTGACAATTAATCATcGGCTCGTATAATGTGTGGAATTGTGAGCGGATAACA
ATTTCACACAGGAAACAGGATCTACCATGGCCGCAGGAAAACCTATCCCAAACCCTCTCCTAGGA
CTGGATTCAACGGGCAGCGGTTCTAGTCTAGCGGCCCCAACTGATCTTCACCAAACGTATTACCG
CCAGGTAAAGAACCCGAATCCGGTGTTCACTCCCCGTGAAGGTGCCGGAACGCTGAAGTTCTGCG
AAAAACTGATGGAAAAGGCGGTGGGCTTCACCTCCCGTTTTGATTTCGCCATTCATGTGGCGCAT
GCCCGTTCCCGTGGTCTGCGTCGGCGCATGCCACCGGTGCTGCGTCGACGGGCTATTGATGCGCT
GCTGCAGGGGCTGTGTTTCCACTATGACCCGCTGGCCAACCGCGTCCAGTGTTCCATCACCACAC
TGGCCATTGAGTGCGGACTGGCGACAGAGTCCGGTGCAGGAAAACTCTCCATCACCCGTGCCACC
CGTGCCCTGACGTTCCTGTCAGAGCTGGGACTGATTACCTACCAGACGGAATATGACCCGCTTAT
CGGGTGCTACATTCCGACCGACATCACGTTCACACTGGCTCTGTTTGCTGCCCTTGATGTGTCTG
AGGATGCAGTGGCAGCTGCGCGCCGCAGTCGTGTTGAATGGGAAAACAAACAGCGCAAAAAGCAG
GGGCTGGATACCCTGGGTATGGATGAGCTGATAGCGAAAGCCTGGCGTTTTGTGCGTGAGCGTTT
CCGCAGTTACCAGACAGAGCTTAAGTCCCGTGGAATAAAACGTGCCCGTGCGCGTCGTGATGCGA
ACAGAGAACGTCAGGATATCGTCACCCTGGTGAAACGGCAGCTGACGCGCGAAATCTCGGAAGGA
CGCTTCACTGCTAATGGTGAGGCGGTAAAACGCGAAGTGGAGCGTCGTGTGAAGGAGCGCATGAT
TCTGTCACGTAACCGCAATTACAGCCGGCTGGCCACAGCTTCTCCCTGAAAGTGATCTCCTCAGA
ATAATCCGGCCTGCGCCGGAGGCATCCGCACGCCTGAAGCCCGCCGGTGCACAAAAAAACAGCGT
CGCATGCAAAAAACAATCTCATCATCCACCTTCTGGAGCATCCGATTCCCCCTGTTTTTAATACA
AAATACGCCTCAGCGACGGGGAATTTTGCTTATCCACATTTAACTGCAAGGGACTTCCCCATAAG
GTTACAACCGTTCATGTCATAAAGCGCCAGCCGCCAGTCTTACAGGGTGCAATGTATCTTTTAAA
CACCTGTTTATATCTCCTTTAAACTACTTAATTACATTCATTTAAAAAGAAAACCTATTCACTGC
CTGTCCTGTGGACAGACAGATATGCACTCGAGCTGACTGATCbioA*C*C
bio-tac-V5-repA-CIS-ori (SEQ ID NO: 7)
bio-
GAACGCGGCTACAATTAATACATAACCCCATCCCCCTGTTGACAATTAATCATcGGCTCGTATAA
TGTGTGGAATTGTGAGCGGATAACAATTTCACACAGGAAACAGGATCTACCATGGCCGCAGGAAA
ACCTATCCCAAACCCTCTCCTAGGACTGGATTCAACGGGCAGCGGTTCTAGTCTAGCGGCCCCAA
CTGATCTTCACCAAACGTATTACCGCCAGGTAAAGAACCCGAATCCGGTGTTCACTCCCCGTGAA
GGTGCCGGAACGCTGAAGTTCTGCGAAAAACTGATGGAAAAGGCGGTGGGCTTCACCTCCCGTTT
TGATTTCGCCATTCATGTGGCGCATGCCCGTTCCCGTGGTCTGCGTCGGCGCATGCCACCGGTGC
TGCGTCGACGGGCTATTGATGCGCTGCTGCAGGGGCTGTGTTTCCACTATGACCCGCTGGCCAAC
CGCGTCCAGTGTTCCATCACCACACTGGCCATTGAGTGCGGACTGGCGACAGAGTCCGGTGCAGG
AAAACTCTCCATCACCCGTGCCACCCGTGCCCTGACGTTCCTGTCAGAGCTGGGACTGATTACCT
ACCAGACGGAATATGACCCGCTTATCGGGTGCTACATTCCGACCGACATCACGTTCACACTGGCT
CTGTTTGCTGCCCTTGATGTGTCTGAGGATGCAGTGGCAGCTGCGCGCCGCAGTCGTGTTGAATG
GGAAAACAAACAGCGCAAAAAGCAGGGGCTGGATACCCTGGGTATGGATGAGCTGATAGCGAAAG
CCTGGCGTTTTGTGCGTGAGCGTTTCCGCAGTTACCAGACAGAGCTTAAGTCCCGTGGAATAAAA
CGTGCCCGTGCGCGTCGTGATGCGAACAGAGAACGTCAGGATATCGTCACCCTGGTGAAACGGCA
GCTGACGCGCGAAATCTCGGAAGGACGCTTCACTGCTAATGGTGAGGCGGTAAAACGCGAAGTGG
AGCGTCGTGTGAAGGAGCGCATGATTCTGTCACGTAACCGCAATTACAGCCGGCTGGCCACAGCT
TCTCCCTGAAAGTGATCTCCTCAGAATAATCCGGCCTGCGCCGGAGGCATCCGCACGCCTGAAGC
CCGCCGGTGCACAAAAAAACAGCGTCGCATGCAAAAAACAATCTCATCATCCACCTTCTGGAGCA
TCCGATTCCCCCTGTTTTTAATACAAAATACGCCTCAGCGACGGGGAATTTTGCTTATCCACATT
TAACTGCAAGGGACTTCCCCATAAGGTTACAACCGTTCATGTCATAAAGCGCCAGCCGCCAGTCT
TACAGGGTGCAATGTATCTTTTAAACACCTGTTTATATCTCCTTTAAACTACTTAATTACATTCA
TTTAAAAAGAAAACCTATTCACTGCCTGTCCTGTGGACAGACAGATATGCA
#144 tach (SEQ ID NO: 8)
CCCCATCCCCCTGTTGACAATTAATC
#472 R1RecForbio (SEQ ID NO: 9)
bio-GAACGCGGCTACAATTAATACATAACC
#85 Orirev (SEQ ID NO: 10)
TGCATATCTGTCTGTCCACAGG
1steprepA (SEQ ID NO: 11)
GGCAGCGGTTCTAGTCTAGCGGCCCCAACTGATCTTCACCAAACGTATTACCGCCAGGTA
AAGAACCCGAATCCGGTGTTCACTCCCCGTGAAGGTGCCGGAACGCCGAAGTTCCGCGAA
AAACCGATGGAAAAGGCGGTGGGCCTCACCTCCCGTTTTGATTTCGCCATTCATGTGGCG
CATGCCCGTTCCCGTGGTCTGCGTCGGCGCATGCCACCGGTGCTGCGTCGACGGGCTATT
GATGCGCTGCTGCAGGGGCTGTGTTTCCACTATGACCCGCTGGCCAACCGCGTCCAGTGT
TCCATCACCACACTGGCCATTGAGTGCGGACTGGCGACAGAGTCCGGTGCAGGAAAACTC
TCCATCACCCGTGCCACCCGGGCCCTGACGTTCCTGTCAGAGCTGGGACTGATTACCTAC
CAGACGGAATATGACCCGCTTATCGGGTGCTACATTCCGACCGACATCACGTTCACACTG
GCTCTGTTTGCTGCCCTTGATGTGTCTGAGGATGCAGTGGCAGCTGCGCGCCGCAGTCGT
GTTGAATGGGAAAACAAACAGCGCAAAAAGCAGGGGCTGGATACCCTGGGTATGGATGAG
CTGATAGCGAAAGCCTGGCGTTTTGTGCGTGAGCGTTTCCGCAGTTACCAGACAGAGCTT
CAGTCCCGTGGAATAAAACGTGCCCGTGCGCGTCGTGATGCGAACAGAGAACGTCAGGAT
ATCGTCACCCTAGTGAAACGGCAGCTGACGCGTGAAATCTCGGAAGGACGCTTCACTGCT
AATGGTGAGGCGGTAAAACGCGAAGTGGAGCGTCGTGTGAAGGAGCGCATGATTCTGTCA
CGTAACCGCAATTACAGCCGGCTGGCCACAGCTTCTCCCTGAAAGTGATCTCCTCAGAAT
AATCCGGCCTGCGCCGGAGGCATCCGCACGCCTGAAGCCCGCCGGTGCACAAAAAAACAG
CGTCGCATGCAAAAAACAATCTCATCATCCACCTTCTGGAGCATCCGATTCCCCCTGTTT
TTAATACAAAATACGCCTCAGCGACGGGGAATTTTGCTTATCCACATTTAACTGCAAGGG
ACTTCCCCATAAGGTTACAACCGTTCATGTCATAAAGCGCCAGCCGCCAGTCTTACAGGG
TGCAATGTATCTTTTAAACACCTGTTTATATCTCCTTTAAACTACTTAATTACATTCATT
TAAAAAGAAAACCTATTCACTGCCTGTCCTGTGGACAGACAGATATGCA
flag-libfor (SEQ ID NO: 12)
ggaaacaggatctaccatggcccagNASNASNASNASNASNASNASNASggcagcggttctagtc
tagc
flaglib-repA-CIS-ori (SEQ ID NO: 13)
GGAAACAGGATCTACCATGGCCCAGNASNASNASNASNASNASNASNASGGCAGCGGTTC
TAGTCTAGCGGCCCCAACTGATCTTCACCAAACGTATTACCGCCAGGTAAAGAACCCGAA
TCCGGTGTTCACTCCCCGTGAAGGTGCCGGAACGCCGAAGTTCCGCGAAAAACCGATGGA
AAAGGCGGTGGGCCTCACCTCCCGTTTTGATTTCGCCATTCATGTGGCGCATGCCCGTTC
CCGTGGTCTGCGTCGGCGCATGCCACCGGTGCTGCGTCGACGGGCTATTGATGCGCTGCT
GCAGGGGCTGTGTTTCCACTATGACCCGCTGGCCAACCGCGTCCAGTGTTCCATCACCAC
ACTGGCCATTGAGTGCGGACTGGCGACAGAGTCCGGTGCAGGAAAACTCTCCATCACCCG
TGCCACCCGGGCCCTGACGTTCCTGTCAGAGCTGGGACTGATTACCTACCAGACGGAATA
TGACCCGCTTATCGGGTGCTACATTCCGACCGACATCACGTTCACACTGGCTCTGTTTGC
TGCCCTTGATGTGTCTGAGGATGCAGTGGCAGCTGCGCGCCGCAGTCGTGTTGAATGGGA
AAACAAACAGCGCAAAAAGCAGGGGCTGGATACCCTGGGTATGGATGAGCTGATAGCGAA
AGCCTGGCGTTTTGTGCGTGAGCGTTTCCGCAGTTACCAGACAGAGCTTCAGTCCCGTGG
AATAAAACGTGCCCGTGCGCGTCGTGATGCGAACAGAGAACGTCAGGATATCGTCACCCT
AGTGAAACGGCAGCTGACGCGTGAAATCTCGGAAGGACGCTTCACTGCTAATGGTGAGGC
GGTAAAACGCGAAGTGGAGCGTCGTGTGAAGGAGCGCATGATTCTGTCACGTAACCGCAA
TTACAGCCGGCTGGCCACAGCTTCTCCCTGAAAGTGATCTCCTCAGAATAATCCGGCCTG
CGCCGGAGGCATCCGCACGCCTGAAGCCCGCCGGTGCACAAAAAAACAGCGTCGCATGCA
AAAAACAATCTCATCATCCACCTTCTGGAGCATCCGATTCCCCCTGTTTTTAATACAAAA
TACGCCTCAGCGACGGGGAATTTTGCTTATCCACATTTAACTGCAAGGGACTTCCCCATA
AGGTTACAACCGTTCATGTCATAAAGCGCCAGCCGCCAGTCTTACAGGGTGCAATGTATC
TTTTAAACACCTGTTTATATCTCCTTTAAACTACTTAATTACATTCATTTAAAAAGAAAA
CCTATTCACTGCCTGTCCTGTGGACAGACAGATATGCA
131-mer (SEQ ID NO: 14)
CGGCGGTTAGAACGCGGCTACAATTAATACATAACCCCATCCCCCTGTTGACAATTAATCATcGG
CTCGTATAATGTGTGGAATTGTGAGCGGATAACAATTTCACACAGGAAACAGGATCTACCATGGC
C
tac-flaglib-repA-CIS-ori (SEQ ID NO: 15)
CGGCGGTTAGAACGCGGCTACAATTAATACATAACCCCATCCCCCTGTTGACAATTAATC
ATCGGCTCGTATAATGTGTGGAATTGTGAGCGGATAACAATTTCACACAGGAAACAGGAT
CTACCATGGCCCAGNASNASNASNASNASNASNASNASGGCAGCGGTTCTAGTCTAGCGG
CCCCAACTGATCTTCACCAAACGTATTACCGCCAGGTAAAGAACCCGAATCCGGTGTTCA
CTCCCCGTGAAGGTGCCGGAACGCCGAAGTTCCGCGAAAAACCGATGGAAAAGGCGGTGG
GCCTCACCTCCCGTTTTGATTTCGCCATTCATGTGGCGCATGCCCGTTCCCGTGGTCTGC
GTCGGCGCATGCCACCGGTGCTGCGTCGACGGGCTATTGATGCGCTGCTGCAGGGGCTGT
GTTTCCACTATGACCCGCTGGCCAACCGCGTCCAGTGTTCCATCACCACACTGGCCATTG
AGTGCGGACTGGCGACAGAGTCCGGTGCAGGAAAACTCTCCATCACCCGTGCCACCCGGG
CCCTGACGTTCCTGTCAGAGCTGGGACTGATTACCTACCAGACGGAATATGACCCGCTTA
TCGGGTGCTACATTCCGACCGACATCACGTTCACACTGGCTCTGTTTGCTGCCCTTGATG
TGTCTGAGGATGCAGTGGCAGCTGCGCGCCGCAGTCGTGTTGAATGGGAAAACAAACAGC
GCAAAAAGCAGGGGCTGGATACCCTGGGTATGGATGAGCTGATAGCGAAAGCCTGGCGTT
TTGTGCGTGAGCGTTTCCGCAGTTACCAGACAGAGCTTCAGTCCCGTGGAATAAAACGTG
CCCGTGCGCGTCGTGATGCGAACAGAGAACGTCAGGATATCGTCACCCTAGTGAAACGGC
AGCTGACGCGTGAAATCTCGGAAGGACGCTTCACTGCTAATGGTGAGGCGGTAAAACGCG
AAGTGGAGCGTCGTGTGAAGGAGCGCATGATTCTGTCACGTAACCGCAATTACAGCCGGC
TGGCCACAGCTTCTCCCTGAAAGTGATCTCCTCAGAATAATCCGGCCTGCGCCGGAGGCA
TCCGCACGCCTGAAGCCCGCCGGTGCACAAAAAAACAGCGTCGCATGCAAAAAACAATCT
CATCATCCACCTTCTGGAGCATCCGATTCCCCCTGTTTTTAATACAAAATACGCCTCAGC
GACGGGGAATTTTGCTTATCCACATTTAACTGCAAGGGACTTCCCCATAAGGTTACAACC
GTTCATGTCATAAAGCGCCAGCCGCCAGTCTTACAGGGTGCAATGTATCTTTTAAACACC
TGTTTATATCTCCTTTAAACTACTTAATTACATTCATTTAAAAAGAAAACCTATTCACTG
CCTGTCCTGTGGACAGACAGATATGCA
Primer A reverse primer (SEQ ID NO: 16)
5′-AATGATACGGCGACCACCGAGATCTACACTCTTTCCCTACACGACGCTCTTCCGA
TCTCgtaggtctcagttggggccgctagactagaacc
Primer B (SEQ ID NO: 17)
5′-CAAGCAGAAGACGGCATACGAGCTCTTCCGATCTCGGCGGTTAGAACGCGGCTAC
Primer C (SEQ ID NO: 18)
AATGATACGGCGACCACCGAGATCTACACTCTTTCCCTACACGACGCTCTTCCGA
TCTCgtaggtctcagttggggccgctagactagaacc
Primer D (SEQ ID NO: 19)
5′-CAAGCAGAAGACGGCATACGAGATCcGTCTCGGCATTCCTGCTGAACCGCTCTT
CCGATCTCGGCGGTTAGAACGCGGCTAC
Oligo C′ (SEQ ID NO: 20)
5′-PS-TTTTTTTTTTAATGATACGGCGACCACCGAGAUCTACAC-3′
Oligo D′ (SEQ ID NO: 21)
5′-PS-TTTTTTTTTTCAAGCAGAAGACGGCATACGAGoxoAT-3′
Read 1 Specific Sequencing Primer (SEQ ID NO: 22)
ACACTCTTTCCCTACACGACGCTCTTCCGATCT
Bsa repfor (SEQ ID NO: 23; BsaI recognition
site shown in capital letters)
aaaGGTCTCccaactgatcttcaccaaacgtattacc
Primer E (SEQ ID NO: 24)
AATGATACGGCGACCACCGAGATCTACACTCTTTCCCTACACGACGCTCTTCCGA
TCTC tgcatatctgtctgtccacagg
Adapter A (SEQ ID NO: 25)
CCATCTCATCCCTGCGTGTCCCATCTGTTCCCTCCCTGTCTCAGCGGCGGTTAGAACGCGGCTAC
Adapter B (SEQ ID NO: 26)
Bio-C₆H₁₄O₄-
CCTATCCCCTGTGTGCCTTGCCTATCCCCTGTTGCGTGTCTCAGtgcatatctgtctgtccacag
g
tac-flaglib-repA-CIS-ori-454adapt (SEQ ID NO: 27)
CCATCTCATCCCTGCGTGTCCCATCTGTTCCCTCCCTGTCTCAGCGGCGGTTAGAACGCG
GCTACAATTAATACATAACCCCATCCCCCTGTTGACAATTAATCATCGGCTCGTATAATG
TGTGGAATTGTGAGCGGATAACAATTTCACACAGGAAACAGGATCTACCATGGCCCAGNA
SNASNASNASNASNASNASNASGGCAGCGGTTCTAGTCTAGCGGCCCCAACTGATCTTCA
CCAAACGTATTACCGCCAGGTAAAGAACCCGAATCCGGTGTTCACTCCCCGTGAAGGTGC
CGGAACGCCGAAGTTCCGCGAAAAACCGATGGAAAAGGCGGTGGGCCTCACCTCCCGTTT
TGATTTCGCCATTCATGTGGCGCATGCCCGTTCCCGTGGTCTGCGTCGGCGCATGCCACC
GGTGCTGCGTCGACGGGCTATTGATGCGCTGCTGCAGGGGCTGTGTTTCCACTATGACCC
GCTGGCCAACCGCGTCCAGTGTTCCATCACCACACTGGCCATTGAGTGCGGACTGGCGAC
AGAGTCCGGTGCAGGAAAACTCTCCATCACCCGTGCCACCCGGGCCCTGACGTTCCTGTC
AGAGCTGGGACTGATTACCTACCAGACGGAATATGACCCGCTTATCGGGTGCTACATTCC
GACCGACATCACGTTCACACTGGCTCTGTTTGCTGCCCTTGATGTGTCTGAGGATGCAGT
GGCAGCTGCGCGCCGCAGTCGTGTTGAATGGGAAAACAAACAGCGCAAAAAGCAGGGGCT
GGATACCCTGGGTATGGATGAGCTGATAGCGAAAGCCTGGCGTTTTGTGCGTGAGCGTTT
CCGCAGTTACCAGACAGAGCTTCAGTCCCGTGGAATAAAACGTGCCCGTGCGCGTCGTGA
TGCGAACAGAGAACGTCAGGATATCGTCACCCTAGTGAAACGGCAGCTGACGCGTGAAAT
CTCGGAAGGACGCTTCACTGCTAATGGTGAGGCGGTAAAACGCGAAGTGGAGCGTCGTGT
GAAGGAGCGCATGATTCTGTCACGTAACCGCAATTACAGCCGGCTGGCCACAGCTTCTCC
CTGAAAGTGATCTCCTCAGAATAATCCGGCCTGCGCCGGAGGCATCCGCACGCCTGAAGC
CCGCCGGTGCACAAAAAAACAGCGTCGCATGCAAAAAACAATCTCATCATCCACCTTCTG
GAGCATCCGATTCCCCCTGTTTTTAATACAAAATACGCCTCAGCGACGGGGAATTTTGCT
TATCCACATTTAACTGCAAGGGACTTCCCCATAAGGTTACAACCGTTCATGTCATAAAGC
GCCAGCCGCCAGTCTTACAGGGTGCAATGTATCTTTTAAACACCTGTTTATATCTCCTTT
AAACTACTTAATTACATTCATTTAAAAAGAAAACCTATTCACTGCCTGTCCTGTGGACAG
ACAGATATGCACTGAGACACGCAACAGGGGATAGGCAAGGCACACAGGGGATAGG
HEG capture primer (-3′; SEQ ID NO: 28)
5′-Amine - (C₁₂H₂₆O₇)₃ -CCTATCCCCTGTGTGCCTTG
454 Seq Forward (SEQ ID NO: 29)
CCATCTCATCCCTGCGTGTC
454 Seq Reverse primers (SEQ ID NO: 30)
CCTATCCCCTGTGTGCCTTG
HEG enrichment primer (SEQ ID NO: 31)
Biotin-C12H26O7-CCATCTCATCCCTGCGTGTCCCATCTGTTCCCTCCCTGTC
Forward primer (Primer A-key): (SEQ ID NO: 32)
5′-CCATCTCATCCCTGCGTGTCTCCGACTCAGCGGCGGTTAGAACGCGGCTAC
Reverse primer (Primer P1-key): (SEQ ID NO: 33)
5′-CCTCTCTATGGGCAGTCGGTGATTGCATATCTGTCTGTCCACAGG
6mer-libfor (SEQ ID NO: 34)
ggaaacaggatctaccatggcccagNNSNNSNNSNNSNNSNNSNNSNNSggcagcggttctagtc
tagc
Pinlibfor (SEQ ID NO: 35)
GGAAACAGGATCTACCATGGCCGATGAAGAGAAACTGCCGCCAGGCTGGNNBAAANNBTGGAGTV
VMVVMGGACGCGTCNNBTACNNBAATNNBATCACTNNBGCGVVMCAGTGGGAACGACCATCGGGC
GGCAGCGGTTCTAGTCTAGC
Oligo D2 (SEQ ID NO: 36; PS represents a phosphorothioate
oligonucleotide; PC represents a photocleavable spacer)
5′-PS-PC-TTTTTTTTTTCAAGCAGAAGACGGCATACGAGoxoAT-3′
OrirevAlex647 (SEQ ID NO: 37)
/5Alex647N/TGCATATCTGTCTGTCCACAGG
tac-flaglib-illmunadapt (SEQ ID NO: 38)
CAAGCAGAAGACGGCATACGAGATCCGTCTCGGCATTCCTGCTGAACCGCTCTTCCGATC
TCGGCGGTTAGAACGCGGCTACAATTAATACATAACCCCATCCCCCTGTTGACAATTAAT
CATCGGCTCGTATAATGTGTGGAATTGTGAGCGGATAACAATTTCACACAGGAAACAGGA
TCTACCATGGCCCAGNASNASNASNASNASNASNASNASGGCAGCGGTTCTAGTCTAGCG
GCCCCAACTGAGACCTACGAGATCGGAAGAGCGTCGTGTAGGGAAAGAGTGTAGATCTCG
GTGGTCGCCGTATCATT
bsarepA-CIS-ori (SEQ ID NO: 39)
AAAGGTCTCCCAACTGATCTTCACCAAACGTATTACCGCCAGGTAAAGAACCCGAATCCG
GTGTTCACTCCCCGTGAAGGTGCCGGAACGCCGAAGTTCCGCGAAAAACCGATGGAAAAG
GCGGTGGGCCTCACCTCCCGTTTTGATTTCGCCATTCATGTGGCGCATGCCCGTTCCCGT
GGTCTGCGTCGGCGCATGCCACCGGTGCTGCGTCGACGGGCTATTGATGCGCTGCTGCAG
GGGCTGTGTTTCCACTATGACCCGCTGGCCAACCGCGTCCAGTGTTCCATCACCACACTG
GCCATTGAGTGCGGACTGGCGACAGAGTCCGGTGCAGGAAAACTCTCCATCACCCGTGCC
ACCCGGGCCCTGACGTTCCTGTCAGAGCTGGGACTGATTACCTACCAGACGGAATATGAC
CCGCTTATCGGGTGCTACATTCCGACCGACATCACGTTCACACTGGCTCTGTTTGCTGCC
CTTGATGTGTCTGAGGATGCAGTGGCAGCTGCGCGCCGCAGTCGTGTTGAATGGGAAAAC
AAACAGCGCAAAAAGCAGGGGCTGGATACCCTGGGTATGGATGAGCTGATAGCGAAAGCC
TGGCGTTTTGTGCGTGAGCGTTTCCGCAGTTACCAGACAGAGCTTCAGTCCCGTGGAATA
AAACGTGCCCGTGCGCGTCGTGATGCGAACAGAGAACGTCAGGATATCGTCACCCTAGTG
AAACGGCAGCTGACGCGTGAAATCTCGGAAGGACGCTTCACTGCTAATGGTGAGGCGGTA
AAACGCGAAGTGGAGCGTCGTGTGAAGGAGCGCATGATTCTGTCACGTAACCGCAATTAC
AGCCGGCTGGCCACAGCTTCTCCCTGAAAGTGATCTCCTCAGAATAATCCGGCCTGCGCC
GGAGGCATCCGCACGCCTGAAGCCCGCCGGTGCACAAAAAAACAGCGTCGCATGCAAAAA
ACAATCTCATCATCCACCTTCTGGAGCATCCGATTCCCCCTGTTTTTAATACAAAATACG
CCTCAGCGACGGGGAATTTTGCTTATCCACATTTAACTGCAAGGGACTTCCCCATAAGGT
TACAACCGTTCATGTCATAAAGCGCCAGCCGCCAGTCTTACAGGGTGCAATGTATCTTTT
AAACACCTGTTTATATCTCCTTTAAACTACTTAATTACATTCATTTAAAAAGAAAACCTA
TTCACTGCCTGTCCTGTGGACAGACAGATATGCA
tac-flaglib-repA-CIS-ori-illumadapt (SEQ ID NO: 40)
CAAGCAGAAGACGGCATACGAGATCCGTCTCGGCATTCCTGCTGAACCGCTCTTCCGATC
TCGGCGGTTAGAACGCGGCTACAATTAATACATAACCCCATCCCCCTGTTGACAATTAAT
CATCGGCTCGTATAATGTGTGGAATTGTGAGCGGATAACAATTTCACACAGGAAACAGGA
TCTACCATGGCCCAGNASNASNASNASNASNASNASNASGGCAGCGGTTCTAGTCTAGCG
GCCCCAACTGATCTTCACCAAACGTATTACCGCCAGGTAAAGAACCCGAATCCGGTGTTC
ACTCCCCGTGAAGGTGCCGGAACGCCGAAGTTCCGCGAAAAACCGATGGAAAAGGCGGTG
GGCCTCACCTCCCGTTTTGATTTCGCCATTCATGTGGCGCATGCCCGTTCCCGTGGTCTG
CGTCGGCGCATGCCACCGGTGCTGCGTCGACGGGCTATTGATGCGCTGCTGCAGGGGCTG
TGTTTCCACTATGACCCGCTGGCCAACCGCGTCCAGTGTTCCATCACCACACTGGCCATT
GAGTGCGGACTGGCGACAGAGTCCGGTGCAGGAAAACTCTCCATCACCCGTGCCACCCGG
GCCCTGACGTTCCTGTCAGAGCTGGGACTGATTACCTACCAGACGGAATATGACCCGCTT
ATCGGGTGCTACATTCCGACCGACATCACGTTCACACTGGCTCTGTTTGCTGCCCTTGAT
GTGTCTGAGGATGCAGTGGCAGCTGCGCGCCGCAGTCGTGTTGAATGGGAAAACAAACAG
CGCAAAAAGCAGGGGCTGGATACCCTGGGTATGGATGAGCTGATAGCGAAAGCCTGGCGT
TTTGTGCGTGAGCGTTTCCGCAGTTACCAGACAGAGCTTCAGTCCCGTGGAATAAAACGT
GCCCGTGCGCGTCGTGATGCGAACAGAGAACGTCAGGATATCGTCACCCTAGTGAAACGG
CAGCTGACGCGTGAAATCTCGGAAGGACGCTTCACTGCTAATGGTGAGGCGGTAAAACGC
GAAGTGGAGCGTCGTGTGAAGGAGCGCATGATTCTGTCACGTAACCGCAATTACAGCCGG
CTGGCCACAGCTTCTCCCTGAAAGTGATCTCCTCAGAATAATCCGGCCTGCGCCGGAGGC
ATCCGCACGCCTGAAGCCCGCCGGTGCACAAAAAAACAGCGTCGCATGCAAAAAACAATC
TCATCATCCACCTTCTGGAGCATCCGATTCCCCCTGTTTTTAATACAAAATACGCCTCAG
CGACGGGGAATTTTGCTTATCCACATTTAACTGCAAGGGACTTCCCCATAAGGTTACAAC
CGTTCATGTCATAAAGCGCCAGCCGCCAGTCTTACAGGGTGCAATGTATCTTTTAAACAC
CTGTTTATATCTCCTTTAAACTACTTAATTACATTCATTTAAAAAGAAAACCTATTCACT
GCCTGTCCTGTGGACAGACAGATATGCAGAGATCGGAAGAGCGTCGTGTAGGGAAAGAGT
GTAGATCTCGGTGGTCGCCGTATCATT
tac-flaglib-repA-CIS-ori-ionadapt (SEQ ID NO: 41)
CCATCTCATCCCTGCGTGTCTCCGACTCAGCGGCGGTTAGAACGCGGCTACAATTAATAC
ATAACCCCATCCCCCTGTTGACAATTAATCATCGGCTCGTATAATGTGTGGAATTGTGAG
CGGATAACAATTTCACACAGGAAACAGGATCTACCATGGCCCAGNASNASNASNASNASN
ASNASNASGGCAGCGGTTCTAGTCTAGCGGCCCCAACTGATCTTCACCAAACGTATTACC
GCCAGGTAAAGAACCCGAATCCGGTGTTCACTCCCCGTGAAGGTGCCGGAACGCCGAAGT
TCCGCGAAAAACCGATGGAAAAGGCGGTGGGCCTCACCTCCCGTTTTGATTTCGCCATTC
ATGTGGCGCATGCCCGTTCCCGTGGTCTGCGTCGGCGCATGCCACCGGTGCTGCGTCGAC
GGGCTATTGATGCGCTGCTGCAGGGGCTGTGTTTCCACTATGACCCGCTGGCCAACCGCG
TCCAGTGTTCCATCACCACACTGGCCATTGAGTGCGGACTGGCGACAGAGTCCGGTGCAG
GAAAACTCTCCATCACCCGTGCCACCCGGGCCCTGACGTTCCTGTCAGAGCTGGGACTGA
TTACCTACCAGACGGAATATGACCCGCTTATCGGGTGCTACATTCCGACCGACATCACGT
TCACACTGGCTCTGTTTGCTGCCCTTGATGTGTCTGAGGATGCAGTGGCAGCTGCGCGCC
GCAGTCGTGTTGAATGGGAAAACAAACAGCGCAAAAAGCAGGGGCTGGATACCCTGGGTA
TGGATGAGCTGATAGCGAAAGCCTGGCGTTTTGTGCGTGAGCGTTTCCGCAGTTACCAGA
CAGAGCTTCAGTCCCGTGGAATAAAACGTGCCCGTGCGCGTCGTGATGCGAACAGAGAAC
GTCAGGATATCGTCACCCTAGTGAAACGGCAGCTGACGCGTGAAATCTCGGAAGGACGCT
TCACTGCTAATGGTGAGGCGGTAAAACGCGAAGTGGAGCGTCGTGTGAAGGAGCGCATGA
TTCTGTCACGTAACCGCAATTACAGCCGGCTGGCCACAGCTTCTCCCTGAAAGTGATCTC
CTCAGAATAATCCGGCCTGCGCCGGAGGCATCCGCACGCCTGAAGCCCGCCGGTGCACAA
AAAAACAGCGTCGCATGCAAAAAACAATCTCATCATCCACCTTCTGGAGCATCCGATTCC
CCCTGTTTTTAATACAAAATACGCCTCAGCGACGGGGAATTTTGCTTATCCACATTTAAC
TGCAAGGGACTTCCCCATAAGGTTACAACCGTTCATGTCATAAAGCGCCAGCCGCCAGTC
TTACAGGGTGCAATGTATCTTTTAAACACCTGTTTATATCTCCTTTAAACTACTTAATTA
CATTCATTTAAAAAGAAAACCTATTCACTGCCTGTCCTGTGGACAGACAGATATGCAATC
ACCGACTGCCCATAGAGAGG
tac-6merlib-repA-CIS-ori (SEQ ID NO: 42)
CGGCGGTTAGAACGCGGCTACAATTAATACATAACCCCATCCCCCTGTTGACAATTAATC
ATCGGCTCGTATAATGTGTGGAATTGTGAGCGGATAACAATTTCACACAGGAAACAGGAT
CTACCATGGCCCAGNASNASNASNASNASNASNASNASGGCAGCGGTTCTAGTCTAGCGG
CCCCAACTGATCTTCACCAAACGTATTACCGCCAGGTAAAGAACCCGAATCCGGTGTTCA
CTCCCCGTGAAGGTGCCGGAACGCCGAAGTTCCGCGAAAAACCGATGGAAAAGGCGGTGG
GCCTCACCTCCCGTTTTGATTTCGCCATTCATGTGGCGCATGCCCGTTCCCGTGGTCTGC
GTCGGCGCATGCCACCGGTGCTGCGTCGACGGGCTATTGATGCGCTGCTGCAGGGGCTGT
GTTTCCACTATGACCCGCTGGCCAACCGCGTCCAGTGTTCCATCACCACACTGGCCATTG
AGTGCGGACTGGCGACAGAGTCCGGTGCAGGAAAACTCTCCATCACCCGTGCCACCCGGG
CCCTGACGTTCCTGTCAGAGCTGGGACTGATTACCTACCAGACGGAATATGACCCGCTTA
TCGGGTGCTACATTCCGACCGACATCACGTTCACACTGGCTCTGTTTGCTGCCCTTGATG
TGTCTGAGGATGCAGTGGCAGCTGCGCGCCGCAGTCGTGTTGAATGGGAAAACAAACAGC
GCAAAAAGCAGGGGCTGGATACCCTGGGTATGGATGAGCTGATAGCGAAAGCCTGGCGTT
TTGTGCGTGAGCGTTTCCGCAGTTACCAGACAGAGCTTCAGTCCCGTGGAATAAAACGTG
CCCGTGCGCGTCGTGATGCGAACAGAGAACGTCAGGATATCGTCACCCTAGTGAAACGGC
AGCTGACGCGTGAAATCTCGGAAGGACGCTTCACTGCTAATGGTGAGGCGGTAAAACGCG
AAGTGGAGCGTCGTGTGAAGGAGCGCATGATTCTGTCACGTAACCGCAATTACAGCCGGC
TGGCCACAGCTTCTCCCTGAAAGTGATCTCCTCAGAATAATCCGGCCTGCGCCGGAGGCA
TCCGCACGCCTGAAGCCCGCCGGTGCACAAAAAAACAGCGTCGCATGCAAAAAACAATCT
CATCATCCACCTTCTGGAGCATCCGATTCCCCCTGTTTTTAATACAAAATACGCCTCAGC
GACGGGGAATTTTGCTTATCCACATTTAACTGCAAGGGACTTCCCCATAAGGTTACAACC
GTTCATGTCATAAAGCGCCAGCCGCCAGTCTTACAGGGTGCAATGTATCTTTTAAACACC
TGTTTATATCTCCTTTAAACTACTTAATTACATTCATTTAAAAAGAAAACCTATTCACTG
CCTGTCCTGTGGACAGACAGATATGCA
tac-6merlib-repA-CIS-ori-illumadapt (SEQ ID NO: 43)
CAAGCAGAAGACGGCATACGAGATCCGTCTCGGCATTCCTGCTGAACCGCTCTTCCGATC
TCGGCGGTTAGAACGCGGCTACAATTAATACATAACCCCATCCCCCTGTTGACAATTAAT
CATCGGCTCGTATAATGTGTGGAATTGTGAGCGGATAACAATTTCACACAGGAAACAGGA
TCTACCATGGCCCAGNNKNNKNNKNNKNNKNNKGGCAGCGGTTCTAGTCTAGCGGCCCCA
ACTGATCTTCACCAAACGTATTACCGCCAGGTAAAGAACCCGAATCCGGTGTTCACTCCC
CGTGAAGGTGCCGGAACGCCGAAGTTCCGCGAAAAACCGATGGAAAAGGCGGTGGGCCTC
ACCTCCCGTTTTGATTTCGCCATTCATGTGGCGCATGCCCGTTCCCGTGGTCTGCGTCGG
CGCATGCCACCGGTGCTGCGTCGACGGGCTATTGATGCGCTGCTGCAGGGGCTGTGTTTC
CACTATGACCCGCTGGCCAACCGCGTCCAGTGTTCCATCACCACACTGGCCATTGAGTGC
GGACTGGCGACAGAGTCCGGTGCAGGAAAACTCTCCATCACCCGTGCCACCCGGGCCCTG
ACGTTCCTGTCAGAGCTGGGACTGATTACCTACCAGACGGAATATGACCCGCTTATCGGG
TGCTACATTCCGACCGACATCACGTTCACACTGGCTCTGTTTGCTGCCCTTGATGTGTCT
GAGGATGCAGTGGCAGCTGCGCGCCGCAGTCGTGTTGAATGGGAAAACAAACAGCGCAAA
AAGCAGGGGCTGGATACCCTGGGTATGGATGAGCTGATAGCGAAAGCCTGGCGTTTTGTG
CGTGAGCGTTTCCGCAGTTACCAGACAGAGCTTCAGTCCCGTGGAATAAAACGTGCCCGT
GCGCGTCGTGATGCGAACAGAGAACGTCAGGATATCGTCACCCTAGTGAAACGGCAGCTG
ACGCGTGAAATCTCGGAAGGACGCTTCACTGCTAATGGTGAGGCGGTAAAACGCGAAGTG
GAGCGTCGTGTGAAGGAGCGCATGATTCTGTCACGTAACCGCAATTACAGCCGGCTGGCC
ACAGCTTCTCCCTGAAAGTGATCTCCTCAGAATAATCCGGCCTGCGCCGGAGGCATCCGC
ACGCCTGAAGCCCGCCGGTGCACAAAAAAACAGCGTCGCATGCAAAAAACAATCTCATCA
TCCACCTTCTGGAGCATCCGATTCCCCCTGTTTTTAATACAAAATACGCCTCAGCGACGG
GGAATTTTGCTTATCCACATTTAACTGCAAGGGACTTCCCCATAAGGTTACAACCGTTCA
TGTCATAAAGCGCCAGCCGCCAGTCTTACAGGGTGCAATGTATCTTTTAAACACCTGTTT
ATATCTCCTTTAAACTACTTAATTACATTCATTTAAAAAGAAAACCTATTCACTGCCTGT
CCTGTGGACAGACAGATATGCAGAGATCGGAAGAGCGTCGTGTAGGGAAAGAGTGTAGAT
CTCGGTGGTCGCCGTATCATT
tac-6merlib-repA-CIS-ori-454adapt (SEQ ID NO: 44)
CCATCTCATCCCTGCGTGTCCCATCTGTTCCCTCCCTGTCTCAGCGGCGGTTAGAACGCG
GCTACAATTAATACATAACCCCATCCCCCTGTTGACAATTAATCATCGGCTCGTATAATG
TGTGGAATTGTGAGCGGATAACAATTTCACACAGGAAACAGGATCTACCATGGCCCAGNA
SNASNASNASNASNASNASNASGGCAGCGGTTCTAGTCTAGCGGCCCCAACTGATCTTCA
CCAAACGTATTACCGCCAGGTAAAGAACCCGAATCCGGTGTTCACTCCCCGTGAAGGTGC
CGGAACGCCGAAGTTCCGCGAAAAACCGATGGAAAAGGCGGTGGGCCTCACCTCCCGTTT
TGATTTCGCCATTCATGTGGCGCATGCCCGTTCCCGTGGTCTGCGTCGGCGCATGCCACC
GGTGCTGCGTCGACGGGCTATTGATGCGCTGCTGCAGGGGCTGTGTTTCCACTATGACCC
GCTGGCCAACCGCGTCCAGTGTTCCATCACCACACTGGCCATTGAGTGCGGACTGGCGAC
AGAGTCCGGTGCAGGAAAACTCTCCATCACCCGTGCCACCCGGGCCCTGACGTTCCTGTC
AGAGCTGGGACTGATTACCTACCAGACGGAATATGACCCGCTTATCGGGTGCTACATTCC
GACCGACATCACGTTCACACTGGCTCTGTTTGCTGCCCTTGATGTGTCTGAGGATGCAGT
GGCAGCTGCGCGCCGCAGTCGTGTTGAATGGGAAAACAAACAGCGCAAAAAGCAGGGGCT
GGATACCCTGGGTATGGATGAGCTGATAGCGAAAGCCTGGCGTTTTGTGCGTGAGCGTTT
CCGCAGTTACCAGACAGAGCTTCAGTCCCGTGGAATAAAACGTGCCCGTGCGCGTCGTGA
TGCGAACAGAGAACGTCAGGATATCGTCACCCTAGTGAAACGGCAGCTGACGCGTGAAAT
CTCGGAAGGACGCTTCACTGCTAATGGTGAGGCGGTAAAACGCGAAGTGGAGCGTCGTGT
GAAGGAGCGCATGATTCTGTCACGTAACCGCAATTACAGCCGGCTGGCCACAGCTTCTCC
CTGAAAGTGATCTCCTCAGAATAATCCGGCCTGCGCCGGAGGCATCCGCACGCCTGAAGC
CCGCCGGTGCACAAAAAAACAGCGTCGCATGCAAAAAACAATCTCATCATCCACCTTCTG
GAGCATCCGATTCCCCCTGTTTTTAATACAAAATACGCCTCAGCGACGGGGAATTTTGCT
TATCCACATTTAACTGCAAGGGACTTCCCCATAAGGTTACAACCGTTCATGTCATAAAGC
GCCAGCCGCCAGTCTTACAGGGTGCAATGTATCTTTTAAACACCTGTTTATATCTCCTTT
AAACTACTTAATTACATTCATTTAAAAAGAAAACCTATTCACTGCCTGTCCTGTGGACAG
ACAGATATGCACTGAGACACGCAACAGGGGATAGGCAAGGCACACAGGGGATAGG
tac-pinlib-repA-CIS-ori (SEQ ID NO: 45)
CGGCGGTTAGAACGCGGCTACAATTAATACATAACCCCATCCCCCTGTTGACAATTAATC
ATCGGCTCGTATAATGTGTGGAATTGTGAGCGGATAACAATTTCACACAGGAAACAGGAT
CTACCATGGCCGATGAAGAGAAACTGCCGCCAGGCTGGNNBAAANNBTGGAGTVVMVVMG
GACGCGTCNNBTACNNBAATNNBATCACTNNBGCGVVMCAGTGGGAACGACCATCGGGCG
GCAGCGGTTCTAGTCTAGCGGCCCCAACTGATCTTCACCAAACGTATTACCGCCAGGTAA
AGAACCCGAATCCGGTGTTCACTCCCCGTGAAGGTGCCGGAACGCCGAAGTTCCGCGAAA
AACCGATGGAAAAGGCGGTGGGCCTCACCTCCCGTTTTGATTTCGCCATTCATGTGGCGC
ATGCCCGTTCCCGTGGTCTGCGTCGGCGCATGCCACCGGTGCTGCGTCGACGGGCTATTG
ATGCGCTGCTGCAGGGGCTGTGTTTCCACTATGACCCGCTGGCCAACCGCGTCCAGTGTT
CCATCACCACACTGGCCATTGAGTGCGGACTGGCGACAGAGTCCGGTGCAGGAAAACTCT
CCATCACCCGTGCCACCCGGGCCCTGACGTTCCTGTCAGAGCTGGGACTGATTACCTACC
AGACGGAATATGACCCGCTTATCGGGTGCTACATTCCGACCGACATCACGTTCACACTGG
CTCTGTTTGCTGCCCTTGATGTGTCTGAGGATGCAGTGGCAGCTGCGCGCCGCAGTCGTG
TTGAATGGGAAAACAAACAGCGCAAAAAGCAGGGGCTGGATACCCTGGGTATGGATGAGC
TGATAGCGAAAGCCTGGCGTTTTGTGCGTGAGCGTTTCCGCAGTTACCAGACAGAGCTTC
AGTCCCGTGGAATAAAACGTGCCCGTGCGCGTCGTGATGCGAACAGAGAACGTCAGGATA
TCGTCACCCTAGTGAAACGGCAGCTGACGCGTGAAATCTCGGAAGGACGCTTCACTGCTA
ATGGTGAGGCGGTAAAACGCGAAGTGGAGCGTCGTGTGAAGGAGCGCATGATTCTGTCAC
GTAACCGCAATTACAGCCGGCTGGCCACAGCTTCTCCCTGAAAGTGATCTCCTCAGAATA
ATCCGGCCTGCGCCGGAGGCATCCGCACGCCTGAAGCCCGCCGGTGCACAAAAAAACAGC
GTCGCATGCAAAAAACAATCTCATCATCCACCTTCTGGAGCATCCGATTCCCCCTGTTTT
TAATACAAAATACGCCTCAGCGACGGGGAATTTTGCTTATCCACATTTAACTGCAAGGGA
CTTCCCCATAAGGTTACAACCGTTCATGTCATAAAGCGCCAGCCGCCAGTCTTACAGGGT
GCAATGTATCTTTTAAACACCTGTTTATATCTCCTTTAAACTACTTAATTACATTCATTT
AAAAAGAAAACCTATTCACTGCCTGTCCTGTGGACAGACAGATATGCA
Pinlibfor (SEQ ID NO: 46)
GCCGATGAAGAGAAACTGCCGCCAGG
Pinlibrev (SEQ ID NO: 47)
CCCGATGGTCGTTCCCACTG
tacP2AHA (SEQ ID NO: 48)
GCTTCAGTAAGCCAGATGCTACACAATTAGGCTTGTACATATTGTCGTTAGAACGCGGCT
ACAATTAATACATAACCTTATGTATCATACACATACGATTTAGGTGACACTATAGAATAC
AAGCTTACTCCCCATCCCCCTGTTGACAATTAATCATGGCTCGTATAATGTGTGGAATTG
TGAGCGGATAACAATTTCACACAGGAAACAGGATCTACCATGGCCGTTAAAGCCTCCGGG
CGTTTTGTCCCTCCGTCAGCATTTGCCGCAGGCACCGGTAAGATGTTTACCGGTGCTTAT
GCATGGAACGCGCCACGGCAGGCCGTCGGGCGCGAAAGACCCCTTACACGTGACGAGATG
CGTCAGATGCAAGGTGTTTTATCCACGATTAACCGCCTGCCTTACTTTTTGCGCTCGCTG
TTTACTTCACGCTATGACTACATCCGGCGCAATAAAAGCCCGGTGCACGGGTTTTATTTC
CTCACATCCACTTTTCAGCGTCGTTTATGGCCGCGCATTGAGCGTGTGAATCAGCGCCAT
GAAATGAACACCGACGCGTCGTTGCTGTTTCTGGCAGAGCGTGACCACTATGCGCGCCTG
CCGGGAATGAATGACAAGGAGCTGAAAAAGTTTGCCGCCCGTATCTCATCGCAGCTTTTC
ATGATGTATGAGGAACTCAGCGATGCCTGGGTGGATGCACATGGCGAAAAAGAATCGCTG
TTTACGGATGAGGCGCAGGCTCACCTCTATGGTCATGTTGCTGGCGCTGCACGTGCTTTC
AATATTTCCCCGCTTTACTGGAAAAAATACCGTAAAGGACAGATGACCACGAGGCAGGCA
TATTCTGCCATTGCCCGTCTGTTTAACGATGAGTGGTGGACTCATCAGCTCAAAGGCCAG
CGTATGCGCTGGCATGAGGCGTTACTGATTGCTGTCGGGGAGGTGAATAAAGACCGTTCT
CCTTATGCCAGTAAACATGCCATTCGTGATGTGCGTGCACGCCGCCAAGCAAATCTGGAA
TTTCTTAAATCGTGTGACCTTGAAAACAGGGAAACCGGCGAGCGCATCGACCTTATCAGT
AAGGTGATGGGCAGTATTTCTAATCCTGAAATTCGCCGGATGGAGCTGATGAACACCATT
GCCGGTATTGAGCGTTACGCCGCCGCAGAGGGTGATGTGGGGATGTTTATCACGCTTACC
GCGCCTTCAAAGTATCACCCGACACGTCAGGTCGGAAAAGGCGAAAGTAAAACCGTCCAG
CTAAATCACGGCTGGAACGATGAGGCATTTAATCCAAAGGATGCGCAGCGTTATCTCTGC
CATATCTGGAGCCTGATGCGCACGGCATTCAAAGATAATGATTTACAGGTCTACGGTTTG
CGTGTCGTCGAGCCACACCACGACGGAACGCCGCACTGGCATATGATGCTTTTTTGTAAT
CCACGCCAGCGTAACCAGATTATCGAAATCATGCGTCGCTATGCGCTCAAAGAGGATGGC
GACGAAAGAGGAGCCGCGCGAAACCGTTTTCAGGCAAAACACCTTAACCAGGGCGGTGCT
GCGGGGTATATCGCGAAATACATCTCAAAAAACATCGATGGCTATGCACTGGATGGTCAG
CTCGATAACGATACCGGCAGACCGCTGAAAGACACTGCTGCGGCTGTTACCGCATGGGCG
TCAACGTGGCGCATCCCACAATTTAAAACGGTTGGTCTGCCGACAATGGGGGCTTACCGT
GAACTACGCAAATTGCCTCGCGGCGTCAGCATTGCTGATGAGTTTGACGAGCGCGTCGAG
GCTGCACGCGCCGCCGCAGACAGTGGTGATTTTGCGTTGTATATCAGCGCGCAGGGTGGG
GCAAATGTCCCGCGCGATTGTCAGACTGTCAGGGTCGCCCGTAGTCCGTCGGATGAGGTT
AACGAGTACGAGGAAGAAGTCGAGAGAGTGGTCGGCATTTACGCGCCGCATCTCGGCGCG
CGTCATATTCATATCACCAGAACGACGGACTGGCGCATTGTGCCGAAAGTTCCGGTCGTT
GAGCCTCTGACTTTAAAAAGCGGCATCGCCGCGCCTCGGAGTCCTGTCAATAACTGTGGA
AAGCTCACCGGTGGTGATACTTCGTTACCGGCTCCCACACCTTCTGAGCACGCCGCAGCA
GTGCTTAATCTGGTTGATGACGGTGTTATTGAATGGAATGAACCGGAGGTCGTGAGGGCG
CTCAGGGGCGCATTAAAATACGACATGAGAACGCCAAACCGTCAGCAAAGAAACGGAAGC
CCGTTAAAACCGCATGAAATTGCACCATCTGCCAGACTGACCAGGTCTGAACGATTGCAG
ATCACCCGTATCCGCGTTGACCTTGCTCAGAACGGTATCAGGCCTCAGCGATGGGAACTT
GAGGCGCTGGCGCGTGGAGCAACCGTAAATTATGACGGGAAAAAATTCACGTATCCGGTC
GCTGATGAGTGGCCGGGATTCTCAACAGTAATGGAGTGGACACTCGAGATGGCTTACCCG
TACGACGTTCCGGACTACGCTCGTTGATAGAATTCATCGAGCCCGCCTAATGAGCGGGCT
TTTTTTTCGATGATATCAGATCTGCCGGTCTCCCTATAGTGAGTCGTATTAATTTCGATA
AGCCAGGTTAACCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCGCGTTG
CTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCC
CTCGTGCGCTCTCCTGTTCCGACCCTGCCGCTTACCGGATACCTGTCCGCCTTTCTCCCT
TCGGGAAGCGTGGCGCTTTCTCAATGCTCACGCTGTAGGTATCTCAGTTCGGTGTA
LAMPS (SEQ ID NO: 49)
TACACCGAACTGAGATACCTAC
LinkP2Afor (SEQ ID NO: 50)
GTTAAAGCCTCCGGGCGTTTTGTCC
P2AAmpF (SEQ ID NO: 51)
GCTTCAGTAAGCCAGATGCTAC
Link-P2A (SEQ ID NO: 52)
GTTAAAGCCTCCGGGCGTTTTGTCCCTCCGTCAGCATTTGCCGCAGGCACCGGTAAGATG
TTTACCGGTGCTTATGCATGGAACGCGCCACGGCAGGCCGTCGGGCGCGAAAGACCCCTT
ACACGTGACGAGATGCGTCAGATGCAAGGTGTTTTATCCACGATTAACCGCCTGCCTTAC
TTTTTGCGCTCGCTGTTTACTTCACGCTATGACTACATCCGGCGCAATAAAAGCCCGGTG
CACGGGTTTTATTTCCTCACATCCACTTTTCAGCGTCGTTTATGGCCGCGCATTGAGCGT
GTGAATCAGCGCCATGAAATGAACACCGACGCGTCGTTGCTGTTTCTGGCAGAGCGTGAC
CACTATGCGCGCCTGCCGGGAATGAATGACAAGGAGCTGAAAAAGTTTGCCGCCCGTATC
TCATCGCAGCTTTTCATGATGTATGAGGAACTCAGCGATGCCTGGGTGGATGCACATGGC
GAAAAAGAATCGCTGTTTACGGATGAGGCGCAGGCTCACCTCTATGGTCATGTTGCTGGC
GCTGCACGTGCTTTCAATATTTCCCCGCTTTACTGGAAAAAATACCGTAAAGGACAGATG
ACCACGAGGCAGGCATATTCTGCCATTGCCCGTCTGTTTAACGATGAGTGGTGGACTCAT
CAGCTCAAAGGCCAGCGTATGCGCTGGCATGAGGCGTTACTGATTGCTGTCGGGGAGGTG
AATAAAGACCGTTCTCCTTATGCCAGTAAACATGCCATTCGTGATGTGCGTGCACGCCGC
CAAGCAAATCTGGAATTTCTTAAATCGTGTGACCTTGAAAACAGGGAAACCGGCGAGCGC
ATCGACCTTATCAGTAAGGTGATGGGCAGTATTTCTAATCCTGAAATTCGCCGGATGGAG
CTGATGAACACCATTGCCGGTATTGAGCGTTACGCCGCCGCAGAGGGTGATGTGGGGATG
TTTATCACGCTTACCGCGCCTTCAAAGTATCACCCGACACGTCAGGTCGGAAAAGGCGAA
AGTAAAACCGTCCAGCTAAATCACGGCTGGAACGATGAGGCATTTAATCCAAAGGATGCG
CAGCGTTATCTCTGCCATATCTGGAGCCTGATGCGCACGGCATTCAAAGATAATGATTTA
CAGGTCTACGGTTTGCGTGTCGTCGAGCCACACCACGACGGAACGCCGCACTGGCATATG
ATGCTTTTTTGTAATCCACGCCAGCGTAACCAGATTATCGAAATCATGCGTCGCTATGCG
CTCAAAGAGGATGGCGACGAAAGAGGAGCCGCGCGAAACCGTTTTCAGGCAAAACACCTT
AACCAGGGCGGTGCTGCGGGGTATATCGCGAAATACATCTCAAAAAACATCGATGGCTAT
GCACTGGATGGTCAGCTCGATAACGATACCGGCAGACCGCTGAAAGACACTGCTGCGGCT
GTTACCGCATGGGCGTCAACGTGGCGCATCCCACAATTTAAAACGGTTGGTCTGCCGACA
ATGGGGGCTTACCGTGAACTACGCAAATTGCCTCGCGGCGTCAGCATTGCTGATGAGTTT
GACGAGCGCGTCGAGGCTGCACGCGCCGCCGCAGACAGTGGTGATTTTGCGTTGTATATC
AGCGCGCAGGGTGGGGCAAATGTCCCGCGCGATTGTCAGACTGTCAGGGTCGCCCGTAGT
CCGTCGGATGAGGTTAACGAGTACGAGGAAGAAGTCGAGAGAGTGGTCGGCATTTACGCG
CCGCATCTCGGCGCGCGTCATATTCATATCACCAGAACGACGGACTGGCGCATTGTGCCG
AAAGTTCCGGTCGTTGAGCCTCTGACTTTAAAAAGCGGCATCGCCGCGCCTCGGAGTCCT
GTCAATAACTGTGGAAAGCTCACCGGTGGTGATACTTCGTTACCGGCTCCCACACCTTCT
GAGCACGCCGCAGCAGTGCTTAATCTGGTTGATGACGGTGTTATTGAATGGAATGAACCG
GAGGTCGTGAGGGCGCTCAGGGGCGCATTAAAATACGACATGAGAACGCCAAACCGTCAG
CAAAGAAACGGAAGCCCGTTAAAACCGCATGAAATTGCACCATCTGCCAGACTGACCAGG
TCTGAACGATTGCAGATCACCCGTATCCGCGTTGACCTTGCTCAGAACGGTATCAGGCCT
CAGCGATGGGAACTTGAGGCGCTGGCGCGTGGAGCAACCGTAAATTATGACGGGAAAAAA
TTCACGTATCCGGTCGCTGATGAGTGGCCGGGATTCTCAACAGTAATGGAGTGGACACTC
GAGATGGCTTACCCGTACGACGTTCCGGACTACGCTCGTTGATAGAATTCATCGAGCCCG
CCTAATGAGCGGGCTTTTTTTTCGATGATATCAGATCTGCCGGTCTCCCTATAGTGAGTC
GTATTAATTTCGATAAGCCAGGTTAACCTGCATTAATGAATCGGCCAACGCGCGGGGAGA
GGCGGTTTGCGTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAA
TCAGGGGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCAATGCTCACGCTGTAGGTATCTC
AGTTCGGTGTA
flaglib-p2afor (SEQ ID NO: 53)
GGAAACAGGATCTACCATGGCCCAGNASNASNASNASNASNASNASNASGTTAAAGCCTC
CGGGCGTTTTGTCCCTCC
flaglib-P2A (SEQ ID NO: 54)
GGAAACAGGATCTACCATGGCCCAGNASNASNASNASNASNASNASNASGTTAAAGCCTC
CGGGCGTTTTGTCCCTCCGTCAGCATTTGCCGCAGGCACCGGTAAGATGTTTACCGGTGC
TTATGCATGGAACGCGCCACGGCAGGCCGTCGGGCGCGAAAGACCCCTTACACGTGACGA
GATGCGTCAGATGCAAGGTGTTTTATCCACGATTAACCGCCTGCCTTACTTTTTGCGCTC
GCTGTTTACTTCACGCTATGACTACATCCGGCGCAATAAAAGCCCGGTGCACGGGTTTTA
TTTCCTCACATCCACTTTTCAGCGTCGTTTATGGCCGCGCATTGAGCGTGTGAATCAGCG
CCATGAAATGAACACCGACGCGTCGTTGCTGTTTCTGGCAGAGCGTGACCACTATGCGCG
CCTGCCGGGAATGAATGACAAGGAGCTGAAAAAGTTTGCCGCCCGTATCTCATCGCAGCT
TTTCATGATGTATGAGGAACTCAGCGATGCCTGGGTGGATGCACATGGCGAAAAAGAATC
GCTGTTTACGGATGAGGCGCAGGCTCACCTCTATGGTCATGTTGCTGGCGCTGCACGTGC
TTTCAATATTTCCCCGCTTTACTGGAAAAAATACCGTAAAGGACAGATGACCACGAGGCA
GGCATATTCTGCCATTGCCCGTCTGTTTAACGATGAGTGGTGGACTCATCAGCTCAAAGG
CCAGCGTATGCGCTGGCATGAGGCGTTACTGATTGCTGTCGGGGAGGTGAATAAAGACCG
TTCTCCTTATGCCAGTAAACATGCCATTCGTGATGTGCGTGCACGCCGCCAAGCAAATCT
GGAATTTCTTAAATCGTGTGACCTTGAAAACAGGGAAACCGGCGAGCGCATCGACCTTAT
CAGTAAGGTGATGGGCAGTATTTCTAATCCTGAAATTCGCCGGATGGAGCTGATGAACAC
CATTGCCGGTATTGAGCGTTACGCCGCCGCAGAGGGTGATGTGGGGATGTTTATCACGCT
TACCGCGCCTTCAAAGTATCACCCGACACGTCAGGTCGGAAAAGGCGAAAGTAAAACCGT
CCAGCTAAATCACGGCTGGAACGATGAGGCATTTAATCCAAAGGATGCGCAGCGTTATCT
CTGCCATATCTGGAGCCTGATGCGCACGGCATTCAAAGATAATGATTTACAGGTCTACGG
TTTGCGTGTCGTCGAGCCACACCACGACGGAACGCCGCACTGGCATATGATGCTTTTTTG
TAATCCACGCCAGCGTAACCAGATTATCGAAATCATGCGTCGCTATGCGCTCAAAGAGGA
TGGCGACGAAAGAGGAGCCGCGCGAAACCGTTTTCAGGCAAAACACCTTAACCAGGGCGG
TGCTGCGGGGTATATCGCGAAATACATCTCAAAAAACATCGATGGCTATGCACTGGATGG
TCAGCTCGATAACGATACCGGCAGACCGCTGAAAGACACTGCTGCGGCTGTTACCGCATG
GGCGTCAACGTGGCGCATCCCACAATTTAAAACGGTTGGTCTGCCGACAATGGGGGCTTA
CCGTGAACTACGCAAATTGCCTCGCGGCGTCAGCATTGCTGATGAGTTTGACGAGCGCGT
CGAGGCTGCACGCGCCGCCGCAGACAGTGGTGATTTTGCGTTGTATATCAGCGCGCAGGG
TGGGGCAAATGTCCCGCGCGATTGTCAGACTGTCAGGGTCGCCCGTAGTCCGTCGGATGA
GGTTAACGAGTACGAGGAAGAAGTCGAGAGAGTGGTCGGCATTTACGCGCCGCATCTCGG
CGCGCGTCATATTCATATCACCAGAACGACGGACTGGCGCATTGTGCCGAAAGTTCCGGT
CGTTGAGCCTCTGACTTTAAAAAGCGGCATCGCCGCGCCTCGGAGTCCTGTCAATAACTG
TGGAAAGCTCACCGGTGGTGATACTTCGTTACCGGCTCCCACACCTTCTGAGCACGCCGC
AGCAGTGCTTAATCTGGTTGATGACGGTGTTATTGAATGGAATGAACCGGAGGTCGTGAG
GGCGCTCAGGGGCGCATTAAAATACGACATGAGAACGCCAAACCGTCAGCAAAGAAACGG
AAGCCCGTTAAAACCGCATGAAATTGCACCATCTGCCAGACTGACCAGGTCTGAACGATT
GCAGATCACCCGTATCCGCGTTGACCTTGCTCAGAACGGTATCAGGCCTCAGCGATGGGA
ACTTGAGGCGCTGGCGCGTGGAGCAACCGTAAATTATGACGGGAAAAAATTCACGTATCC
GGTCGCTGATGAGTGGCCGGGATTCTCAACAGTAATGGAGTGGACACTCGAGATGGCTTA
CCCGTACGACGTTCCGGACTACGCTCGTTGATAGAATTCATCGAGCCCGCCTAATGAGCG
GGCTTTTTTTTCGATGATATCAGATCTGCCGGTCTCCCTATAGTGAGTCGTATTAATTTC
GATAAGCCAGGTTAACCTGCATTAATGAATCGGCCAACGCGCGGGGAGAGGCGGTTTGCG
TATTGGGCGCTCTTCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTC
AAGTCAGAGGTGGCGAAACCCGACAGGACTATAAAGATACCAGGCGTTTCCCCCTGGAAG
CTCCCTCGTGCGCTCTCCTGTTCCGACCCTGCCGCTTACCGGATACCTGTCCGCCTTTCT
CCCTTCGGGAAGCGTGGCGCTTTCTCAATGCTCACGCTGTAGGTATCTCAGTTCGGTGTA
tacflaglib-P2A (SEQ ID NO: 55)
CGGCGGTTAGAACGCGGCTACAATTAATACATAACCCCATCCCCCTGTTGACAATTAATC
ATCGGCTCGTATAATGTGTGGAATTGTGAGCGGATAACAATTTCACACAGGAAACAGGAT
CTACCATGGCCCAGNASNASNASNASNASNASNASNASGTTAAAGCCTCCGGGCGTTTTG
TCCCTCCGTCAGCATTTGCCGCAGGCACCGGTAAGATGTTTACCGGTGCTTATGCATGGA
ACGCGCCACGGCAGGCCGTCGGGCGCGAAAGACCCCTTACACGTGACGAGATGCGTCAGA
TGCAAGGTGTTTTATCCACGATTAACCGCCTGCCTTACTTTTTGCGCTCGCTGTTTACTT
CACGCTATGACTACATCCGGCGCAATAAAAGCCCGGTGCACGGGTTTTATTTCCTCACAT
CCACTTTTCAGCGTCGTTTATGGCCGCGCATTGAGCGTGTGAATCAGCGCCATGAAATGA
ACACCGACGCGTCGTTGCTGTTTCTGGCAGAGCGTGACCACTATGCGCGCCTGCCGGGAA
TGAATGACAAGGAGCTGAAAAAGTTTGCCGCCCGTATCTCATCGCAGCTTTTCATGATGT
ATGAGGAACTCAGCGATGCCTGGGTGGATGCACATGGCGAAAAAGAATCGCTGTTTACGG
ATGAGGCGCAGGCTCACCTCTATGGTCATGTTGCTGGCGCTGCACGTGCTTTCAATATTT
CCCCGCTTTACTGGAAAAAATACCGTAAAGGACAGATGACCACGAGGCAGGCATATTCTG
CCATTGCCCGTCTGTTTAACGATGAGTGGTGGACTCATCAGCTCAAAGGCCAGCGTATGC
GCTGGCATGAGGCGTTACTGATTGCTGTCGGGGAGGTGAATAAAGACCGTTCTCCTTATG
CCAGTAAACATGCCATTCGTGATGTGCGTGCACGCCGCCAAGCAAATCTGGAATTTCTTA
AATCGTGTGACCTTGAAAACAGGGAAACCGGCGAGCGCATCGACCTTATCAGTAAGGTGA
TGGGCAGTATTTCTAATCCTGAAATTCGCCGGATGGAGCTGATGAACACCATTGCCGGTA
TTGAGCGTTACGCCGCCGCAGAGGGTGATGTGGGGATGTTTATCACGCTTACCGCGCCTT
CAAAGTATCACCCGACACGTCAGGTCGGAAAAGGCGAAAGTAAAACCGTCCAGCTAAATC
ACGGCTGGAACGATGAGGCATTTAATCCAAAGGATGCGCAGCGTTATCTCTGCCATATCT
GGAGCCTGATGCGCACGGCATTCAAAGATAATGATTTACAGGTCTACGGTTTGCGTGTCG
TCGAGCCACACCACGACGGAACGCCGCACTGGCATATGATGCTTTTTTGTAATCCACGCC
AGCGTAACCAGATTATCGAAATCATGCGTCGCTATGCGCTCAAAGAGGATGGCGACGAAA
GAGGAGCCGCGCGAAACCGTTTTCAGGCAAAACACCTTAACCAGGGCGGTGCTGCGGGGT
ATATCGCGAAATACATCTCAAAAAACATCGATGGCTATGCACTGGATGGTCAGCTCGATA
ACGATACCGGCAGACCGCTGAAAGACACTGCTGCGGCTGTTACCGCATGGGCGTCAACGT
GGCGCATCCCACAATTTAAAACGGTTGGTCTGCCGACAATGGGGGCTTACCGTGAACTAC
GCAAATTGCCTCGCGGCGTCAGCATTGCTGATGAGTTTGACGAGCGCGTCGAGGCTGCAC
GCGCCGCCGCAGACAGTGGTGATTTTGCGTTGTATATCAGCGCGCAGGGTGGGGCAAATG
TCCCGCGCGATTGTCAGACTGTCAGGGTCGCCCGTAGTCCGTCGGATGAGGTTAACGAGT
ACGAGGAAGAAGTCGAGAGAGTGGTCGGCATTTACGCGCCGCATCTCGGCGCGCGTCATA
TTCATATCACCAGAACGACGGACTGGCGCATTGTGCCGAAAGTTCCGGTCGTTGAGCCTC
TGACTTTAAAAAGCGGCATCGCCGCGCCTCGGAGTCCTGTCAATAACTGTGGAAAGCTCA
CCGGTGGTGATACTTCGTTACCGGCTCCCACACCTTCTGAGCACGCCGCAGCAGTGCTTA
ATCTGGTTGATGACGGTGTTATTGAATGGAATGAACCGGAGGTCGTGAGGGCGCTCAGGG
GCGCATTAAAATACGACATGAGAACGCCAAACCGTCAGCAAAGAAACGGAAGCCCGTTAA
AACCGCATGAAATTGCACCATCTGCCAGACTGACCAGGTCTGAACGATTGCAGATCACCC
GTATCCGCGTTGACCTTGCTCAGAACGGTATCAGGCCTCAGCGATGGGAACTTGAGGCGC
TGGCGCGTGGAGCAACCGTAAATTATGACGGGAAAAAATTCACGTATCCGGTCGCTGATG
AGTGGCCGGGATTCTCAACAGTAATGGAGTGGACACTCGAGATGGCTTACCCGTACGACG
TTCCGGACTACGCTCGTTGATAGAATTCATCGAGCCCGCCTAATGAGCGGGCTTTTTTTT
CGATGATATCAGATCTGCCGGTCTCCCTATAGTGAGTCGTATTAATTTCGATAAGCCAGG
TTAACCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGA
ACATGTGAGCAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAA
GCGTGGCGCTTTCTCAATGCTCACGCTGTAGGTATCTCAGTTCGGTGTA
Adapter C (SEQ ID NO: 56)
BioTEG-
CCTATCCCCTGTGTGCCTTGCCTATCCCCTGTTGCGTGTCTCAtacaccgaactgagatacctac
agcgtg
tac-flaglib-P2A-454-adapted (SEQ ID NO: 57)
CCATCTCATCCCTGCGTGTCCCATCTGTTCCCTCCCTGTCTCAGCGGCGGTTAGAACGCG
GCTACAATTAATACATAACCCCATCCCCCTGTTGACAATTAATCATCGGCTCGTATAATG
TGTGGAATTGTGAGCGGATAACAATTTCACACAGGAAACAGGATCTACCATGGCCCAGNA
SNASNASNASNASNASNASNASGTTAAAGCCTCCGGGCGTTTTGTCCCTCCGTCAGCATT
TGCCGCAGGCACCGGTAAGATGTTTACCGGTGCTTATGCATGGAACGCGCCACGGCAGGC
CGTCGGGCGCGAAAGACCCCTTACACGTGACGAGATGCGTCAGATGCAAGGTGTTTTATC
CACGATTAACCGCCTGCCTTACTTTTTGCGCTCGCTGTTTACTTCACGCTATGACTACAT
CCGGCGCAATAAAAGCCCGGTGCACGGGTTTTATTTCCTCACATCCACTTTTCAGCGTCG
TTTATGGCCGCGCATTGAGCGTGTGAATCAGCGCCATGAAATGAACACCGACGCGTCGTT
GCTGTTTCTGGCAGAGCGTGACCACTATGCGCGCCTGCCGGGAATGAATGACAAGGAGCT
GAAAAAGTTTGCCGCCCGTATCTCATCGCAGCTTTTCATGATGTATGAGGAACTCAGCGA
TGCCTGGGTGGATGCACATGGCGAAAAAGAATCGCTGTTTACGGATGAGGCGCAGGCTCA
CCTCTATGGTCATGTTGCTGGCGCTGCACGTGCTTTCAATATTTCCCCGCTTTACTGGAA
AAAATACCGTAAAGGACAGATGACCACGAGGCAGGCATATTCTGCCATTGCCCGTCTGTT
TAACGATGAGTGGTGGACTCATCAGCTCAAAGGCCAGCGTATGCGCTGGCATGAGGCGTT
ACTGATTGCTGTCGGGGAGGTGAATAAAGACCGTTCTCCTTATGCCAGTAAACATGCCAT
TCGTGATGTGCGTGCACGCCGCCAAGCAAATCTGGAATTTCTTAAATCGTGTGACCTTGA
AAACAGGGAAACCGGCGAGCGCATCGACCTTATCAGTAAGGTGATGGGCAGTATTTCTAA
TCCTGAAATTCGCCGGATGGAGCTGATGAACACCATTGCCGGTATTGAGCGTTACGCCGC
CGCAGAGGGTGATGTGGGGATGTTTATCACGCTTACCGCGCCTTCAAAGTATCACCCGAC
ACGTCAGGTCGGAAAAGGCGAAAGTAAAACCGTCCAGCTAAATCACGGCTGGAACGATGA
GGCATTTAATCCAAAGGATGCGCAGCGTTATCTCTGCCATATCTGGAGCCTGATGCGCAC
GGCATTCAAAGATAATGATTTACAGGTCTACGGTTTGCGTGTCGTCGAGCCACACCACGA
CGGAACGCCGCACTGGCATATGATGCTTTTTTGTAATCCACGCCAGCGTAACCAGATTAT
CGAAATCATGCGTCGCTATGCGCTCAAAGAGGATGGCGACGAAAGAGGAGCCGCGCGAAA
CCGTTTTCAGGCAAAACACCTTAACCAGGGCGGTGCTGCGGGGTATATCGCGAAATACAT
CTCAAAAAACATCGATGGCTATGCACTGGATGGTCAGCTCGATAACGATACCGGCAGACC
GCTGAAAGACACTGCTGCGGCTGTTACCGCATGGGCGTCAACGTGGCGCATCCCACAATT
TAAAACGGTTGGTCTGCCGACAATGGGGGCTTACCGTGAACTACGCAAATTGCCTCGCGG
CGTCAGCATTGCTGATGAGTTTGACGAGCGCGTCGAGGCTGCACGCGCCGCCGCAGACAG
TGGTGATTTTGCGTTGTATATCAGCGCGCAGGGTGGGGCAAATGTCCCGCGCGATTGTCA
GACTGTCAGGGTCGCCCGTAGTCCGTCGGATGAGGTTAACGAGTACGAGGAAGAAGTCGA
GAGAGTGGTCGGCATTTACGCGCCGCATCTCGGCGCGCGTCATATTCATATCACCAGAAC
GACGGACTGGCGCATTGTGCCGAAAGTTCCGGTCGTTGAGCCTCTGACTTTAAAAAGCGG
CATCGCCGCGCCTCGGAGTCCTGTCAATAACTGTGGAAAGCTCACCGGTGGTGATACTTC
GTTACCGGCTCCCACACCTTCTGAGCACGCCGCAGCAGTGCTTAATCTGGTTGATGACGG
TGTTATTGAATGGAATGAACCGGAGGTCGTGAGGGCGCTCAGGGGCGCATTAAAATACGA
CATGAGAACGCCAAACCGTCAGCAAAGAAACGGAAGCCCGTTAAAACCGCATGAAATTGC
ACCATCTGCCAGACTGACCAGGTCTGAACGATTGCAGATCACCCGTATCCGCGTTGACCT
TGCTCAGAACGGTATCAGGCCTCAGCGATGGGAACTTGAGGCGCTGGCGCGTGGAGCAAC
CGTAAATTATGACGGGAAAAAATTCACGTATCCGGTCGCTGATGAGTGGCCGGGATTCTC
AACAGTAATGGAGTGGACACTCGAGATGGCTTACCCGTACGACGTTCCGGACTACGCTCG
TTGATAGAATTCATCGAGCCCGCCTAATGAGCGGGCTTTTTTTTCGATGATATCAGATCT
GCCGGTCTCCCTATAGTGAGTCGTATTAATTTCGATAAGCCAGGTTAACCTGCATTAATG
AATCGGCCAACGCGCGGGGAGAGGCGGTTTGCGTATTGGGCGCTCTTCCGCTTCCTCGCT
CACTGACTCGCTGCGCTCGGTCGTTCGGCTGCGGCGAGCGGTATCAGCTCACTCAAAGGC
GGTAATACGGTTATCCACAGAATCAGGGGATAACGCAGGAAAGAACATGTGAGCAAAAGG
CCAGCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCTCA
ATGCTCACGCTGTAGGTATCTCAGTTCGGTGTATGAGACACGCAACAGGGGATAGGCAAG
GCACACAGGGGATAGG
R1-ori sequence (SEQ ID NO: 58)
TTATCCACATTTAACTGCAAGGGACTTCCCCATAAGGTTACAACCGTTCATGTCATAAAGCGCCA
GCCGCCAGTCTTACAGGGTGCAATGTATCTTTTAAACACCTGTTTATATCTCC
R100-ori sequence (SEQ ID NO: 59)
TTATCCACATTAAACTGCAAGGGACTTCCCCATAAGGTTACAACCGTTCATGTCATAAAGCGCCA
TCCGCCAGCGTTACAGGGTGCAATGTATCTTTTAAACACCTGTTTATATCTCC
P2A ori (SEQ ID NO: 60)
GCGCCTCGGAGTCCTGTCAA
Amino acid linker (SEQ ID NO: 61)
GSGSS
15mer-lib1for (SEQ ID NO: 62)
ggaaacaggatctaccatggcccagYACSCGATSRACRACYTGYTGRACYACSTTSTTSCGARAM
TGCRTggcagcggttctagtctagc
15mer-lib2for (SEQ ID NO: 63)
GGAAACAGGATCTACCATGGCCGATGAAGAGAAACTGCCGCCAGGCTGGSCGGYACSCGATSRAC
RACYTGYTGRACYACSTTSTTSCGARAMTGCRTCAGTGGGAACGACCATCGGGCGGCAGCGGTTC
TAGTCTAGC
tac-15merlib1-repA-CIS-ori (SEQ ID NO: 64)
CGGCGGTTAGAACGCGGCTACAATTAATACATAACCCCATCCCCCTGTTGACAATTAATC
ATCGGCTCGTATAATGTGTGGAATTGTGAGCGGATAACAATTTCACACAGGAAACAGGAT
CTACCATGGCCCAGYACSCGATSRACRACYTGYTGRACYACSTTSTTSCGARAMTGCRTG
GCAGCGGTTCTAGTCTAGCGGCCCCAACTGATCTTCACCAAACGTATTACCGCCAGGTAA
AGAACCCGAATCCGGTGTTCACTCCCCGTGAAGGTGCCGGAACGCCGAAGTTCCGCGAAA
AACCGATGGAAAAGGCGGTGGGCCTCACCTCCCGTTTTGATTTCGCCATTCATGTGGCGC
ATGCCCGTTCCCGTGGTCTGCGTCGGCGCATGCCACCGGTGCTGCGTCGACGGGCTATTG
ATGCGCTGCTGCAGGGGCTGTGTTTCCACTATGACCCGCTGGCCAACCGCGTCCAGTGTT
CCATCACCACACTGGCCATTGAGTGCGGACTGGCGACAGAGTCCGGTGCAGGAAAACTCT
CCATCACCCGTGCCACCCGGGCCCTGACGTTCCTGTCAGAGCTGGGACTGATTACCTACC
AGACGGAATATGACCCGCTTATCGGGTGCTACATTCCGACCGACATCACGTTCACACTGG
CTCTGTTTGCTGCCCTTGATGTGTCTGAGGATGCAGTGGCAGCTGCGCGCCGCAGTCGTG
TTGAATGGGAAAACAAACAGCGCAAAAAGCAGGGGCTGGATACCCTGGGTATGGATGAGC
TGATAGCGAAAGCCTGGCGTTTTGTGCGTGAGCGTTTCCGCAGTTACCAGACAGAGCTTC
AGTCCCGTGGAATAAAACGTGCCCGTGCGCGTCGTGATGCGAACAGAGAACGTCAGGATA
TCGTCACCCTAGTGAAACGGCAGCTGACGCGTGAAATCTCGGAAGGACGCTTCACTGCTA
ATGGTGAGGCGGTAAAACGCGAAGTGGAGCGTCGTGTGAAGGAGCGCATGATTCTGTCAC
GTAACCGCAATTACAGCCGGCTGGCCACAGCTTCTCCCTGAAAGTGATCTCCTCAGAATA
ATCCGGCCTGCGCCGGAGGCATCCGCACGCCTGAAGCCCGCCGGTGCACAAAAAAACAGC
GTCGCATGCAAAAAACAATCTCATCATCCACCTTCTGGAGCATCCGATTCCCCCTGTTTT
TAATACAAAATACGCCTCAGCGACGGGGAATTTTGCTTATCCACATTTAACTGCAAGGGA
CTTCCCCATAAGGTTACAACCGTTCATGTCATAAAGCGCCAGCCGCCAGTCTTACAGGGT
GCAATGTATCTTTTAAACACCTGTTTATATCTCCTTTAAACTACTTAATTACATTCATTT
AAAAAGAAAACCTATTCACTGCCTGTCCTGTGGACAGACAGATATGCA
tac-15merlib1-repA-CIS-ori-illumadapt (SEQ ID NO: 65)
CAAGCAGAAGACGGCATACGAGATCCGTCTCGGCATTCCTGCTGAACCGCTCTTCCGATC
TCGGCGGTTAGAACGCGGCTACAATTAATACATAACCCCATCCCCCTGTTGACAATTAAT
CATCGGCTCGTATAATGTGTGGAATTGTGAGCGGATAACAATTTCACACAGGAAACAGGA
TCTACCATGGCCCAGYACSCGATSRACRACYTGYTGRACYACSTTSTTSCGARAMTGCRT
GGCAGCGGTTCTAGTCTAGCGGCCCCAACTGATCTTCACCAAACGTATTACCGCCAGGTA
AAGAACCCGAATCCGGTGTTCACTCCCCGTGAAGGTGCCGGAACGCCGAAGTTCCGCGAA
AAACCGATGGAAAAGGCGGTGGGCCTCACCTCCCGTTTTGATTTCGCCATTCATGTGGCG
CATGCCCGTTCCCGTGGTCTGCGTCGGCGCATGCCACCGGTGCTGCGTCGACGGGCTATT
GATGCGCTGCTGCAGGGGCTGTGTTTCCACTATGACCCGCTGGCCAACCGCGTCCAGTGT
TCCATCACCACACTGGCCATTGAGTGCGGACTGGCGACAGAGTCCGGTGCAGGAAAACTC
TCCATCACCCGTGCCACCCGGGCCCTGACGTTCCTGTCAGAGCTGGGACTGATTACCTAC
CAGACGGAATATGACCCGCTTATCGGGTGCTACATTCCGACCGACATCACGTTCACACTG
GCTCTGTTTGCTGCCCTTGATGTGTCTGAGGATGCAGTGGCAGCTGCGCGCCGCAGTCGT
GTTGAATGGGAAAACAAACAGCGCAAAAAGCAGGGGCTGGATACCCTGGGTATGGATGAG
CTGATAGCGAAAGCCTGGCGTTTTGTGCGTGAGCGTTTCCGCAGTTACCAGACAGAGCTT
CAGTCCCGTGGAATAAAACGTGCCCGTGCGCGTCGTGATGCGAACAGAGAACGTCAGGAT
ATCGTCACCCTAGTGAAACGGCAGCTGACGCGTGAAATCTCGGAAGGACGCTTCACTGCT
AATGGTGAGGCGGTAAAACGCGAAGTGGAGCGTCGTGTGAAGGAGCGCATGATTCTGTCA
CGTAACCGCAATTACAGCCGGCTGGCCACAGCTTCTCCCTGAAAGTGATCTCCTCAGAAT
AATCCGGCCTGCGCCGGAGGCATCCGCACGCCTGAAGCCCGCCGGTGCACAAAAAAACAG
CGTCGCATGCAAAAAACAATCTCATCATCCACCTTCTGGAGCATCCGATTCCCCCTGTTT
TTAATACAAAATACGCCTCAGCGACGGGGAATTTTGCTTATCCACATTTAACTGCAAGGG
ACTTCCCCATAAGGTTACAACCGTTCATGTCATAAAGCGCCAGCCGCCAGTCTTACAGGG
TGCAATGTATCTTTTAAACACCTGTTTATATCTCCTTTAAACTACTTAATTACATTCATT
TAAAAAGAAAACCTATTCACTGCCTGTCCTGTGGACAGACAGATATGCAGAGATCGGAAG
AGCGTCGTGTAGGGAAAGAGTGTAGATCTCGGTGGTCGCCGTATCATT
tac-15merlib1-repA-CIS-ori-454adapt (SEQ ID NO: 66)
CCATCTCATCCCTGCGTGTCCCATCTGTTCCCTCCCTGTCTCAGCGGCGGTTAGAACGCG
GCTACAATTAATACATAACCCCATCCCCCTGTTGACAATTAATCATCGGCTCGTATAATG
TGTGGAATTGTGAGCGGATAACAATTTCACACAGGAAACAGGATCTACCATGGCCCAGYA
CSCGATSRACRACYTGYTGRACYACSTTSTTSCGARAMTGCRTGGCAGCGGTTCTAGTCT
AGCGGCCCCAACTGATCTTCACCAAACGTATTACCGCCAGGTAAAGAACCCGAATCCGGT
GTTCACTCCCCGTGAAGGTGCCGGAACGCCGAAGTTCCGCGAAAAACCGATGGAAAAGGC
GGTGGGCCTCACCTCCCGTTTTGATTTCGCCATTCATGTGGCGCATGCCCGTTCCCGTGG
TCTGCGTCGGCGCATGCCACCGGTGCTGCGTCGACGGGCTATTGATGCGCTGCTGCAGGG
GCTGTGTTTCCACTATGACCCGCTGGCCAACCGCGTCCAGTGTTCCATCACCACACTGGC
CATTGAGTGCGGACTGGCGACAGAGTCCGGTGCAGGAAAACTCTCCATCACCCGTGCCAC
CCGGGCCCTGACGTTCCTGTCAGAGCTGGGACTGATTACCTACCAGACGGAATATGACCC
GCTTATCGGGTGCTACATTCCGACCGACATCACGTTCACACTGGCTCTGTTTGCTGCCCT
TGATGTGTCTGAGGATGCAGTGGCAGCTGCGCGCCGCAGTCGTGTTGAATGGGAAAACAA
ACAGCGCAAAAAGCAGGGGCTGGATACCCTGGGTATGGATGAGCTGATAGCGAAAGCCTG
GCGTTTTGTGCGTGAGCGTTTCCGCAGTTACCAGACAGAGCTTCAGTCCCGTGGAATAAA
ACGTGCCCGTGCGCGTCGTGATGCGAACAGAGAACGTCAGGATATCGTCACCCTAGTGAA
ACGGCAGCTGACGCGTGAAATCTCGGAAGGACGCTTCACTGCTAATGGTGAGGCGGTAAA
ACGCGAAGTGGAGCGTCGTGTGAAGGAGCGCATGATTCTGTCACGTAACCGCAATTACAG
CCGGCTGGCCACAGCTTCTCCCTGAAAGTGATCTCCTCAGAATAATCCGGCCTGCGCCGG
AGGCATCCGCACGCCTGAAGCCCGCCGGTGCACAAAAAAACAGCGTCGCATGCAAAAAAC
AATCTCATCATCCACCTTCTGGAGCATCCGATTCCCCCTGTTTTTAATACAAAATACGCC
TCAGCGACGGGGAATTTTGCTTATCCACATTTAACTGCAAGGGACTTCCCCATAAGGTTA
CAACCGTTCATGTCATAAAGCGCCAGCCGCCAGTCTTACAGGGTGCAATGTATCTTTTAA
ACACCTGTTTATATCTCCTTTAAACTACTTAATTACATTCATTTAAAAAGAAAACCTATT
CACTGCCTGTCCTGTGGACAGACAGATATGCACTGAGACACGCAACAGGGGATAGGCAAG
GCACACAGGGGATAGG
tac-15merlib2-repA-CIS-ori (SEQ ID NO: 67)
CGGCGGTTAGAACGCGGCTACAATTAATACATAACCCCATCCCCCTGTTGACAATTAATC
ATCGGCTCGTATAATGTGTGGAATTGTGAGCGGATAACAATTTCACACAGGAAACAGGAT
CTACCATGGCCGATGAAGAGAAACTGCCGCCAGGCTGGYACSCGATSRACRACYTGYTGR
ACYACSTTSTTSCGARAMTGCRTCAGTGGGAACGACCATCGGGCGGCAGCGGTTCTAGTC
TAGCGGCCCCAACTGATCTTCACCAAACGTATTACCGCCAGGTAAAGAACCCGAATCCGG
TGTTCACTCCCCGTGAAGGTGCCGGAACGCCGAAGTTCCGCGAAAAACCGATGGAAAAGG
CGGTGGGCCTCACCTCCCGTTTTGATTTCGCCATTCATGTGGCGCATGCCCGTTCCCGTG
GTCTGCGTCGGCGCATGCCACCGGTGCTGCGTCGACGGGCTATTGATGCGCTGCTGCAGG
GGCTGTGTTTCCACTATGACCCGCTGGCCAACCGCGTCCAGTGTTCCATCACCACACTGG
CCATTGAGTGCGGACTGGCGACAGAGTCCGGTGCAGGAAAACTCTCCATCACCCGTGCCA
CCCGGGCCCTGACGTTCCTGTCAGAGCTGGGACTGATTACCTACCAGACGGAATATGACC
CGCTTATCGGGTGCTACATTCCGACCGACATCACGTTCACACTGGCTCTGTTTGCTGCCC
TTGATGTGTCTGAGGATGCAGTGGCAGCTGCGCGCCGCAGTCGTGTTGAATGGGAAAACA
AACAGCGCAAAAAGCAGGGGCTGGATACCCTGGGTATGGATGAGCTGATAGCGAAAGCCT
GGCGTTTTGTGCGTGAGCGTTTCCGCAGTTACCAGACAGAGCTTCAGTCCCGTGGAATAA
AACGTGCCCGTGCGCGTCGTGATGCGAACAGAGAACGTCAGGATATCGTCACCCTAGTGA
AACGGCAGCTGACGCGTGAAATCTCGGAAGGACGCTTCACTGCTAATGGTGAGGCGGTAA
AACGCGAAGTGGAGCGTCGTGTGAAGGAGCGCATGATTCTGTCACGTAACCGCAATTACA
GCCGGCTGGCCACAGCTTCTCCCTGAAAGTGATCTCCTCAGAATAATCCGGCCTGCGCCG
GAGGCATCCGCACGCCTGAAGCCCGCCGGTGCACAAAAAAACAGCGTCGCATGCAAAAAA
CAATCTCATCATCCACCTTCTGGAGCATCCGATTCCCCCTGTTTTTAATACAAAATACGC
CTCAGCGACGGGGAATTTTGCTTATCCACATTTAACTGCAAGGGACTTCCCCATAAGGTT
ACAACCGTTCATGTCATAAAGCGCCAGCCGCCAGTCTTACAGGGTGCAATGTATCTTTTA
AACACCTGTTTATATCTCCTTTAAACTACTTAATTACATTCATTTAAAAAGAAAACCTAT
TCACTGCCTGTCCTGTGGACAGACAGATATGCA
15merlib2-recoveryfor (SEQ ID NO: 68)
GCCGATGAAGAGAAACTGCCGCCAGG
15merlib2-recoveryrev (SEQ ID NO: 69)
CCCGATGGTCGTTCCCACTG

1. A method for identifying a member of a peptide library that interacts with a target molecule in situ, the method comprising:
(a) providing a plurality of nucleic acid molecules each encoding a member of the peptide library;
(b) immobilising the plurality of nucleic acid molecules on a solid support;
(c) sequencing the plurality of nucleic acid molecules in situ on the solid support;
(d) expressing the immobilised nucleic acid molecules to produce the peptide library, wherein each member of the peptide library is immobilised on the nucleic acid molecule from which it was expressed;
(e) contacting the immobilised peptide library with the target molecule;
(f) detecting an interaction between at least one member of the peptide library and the target molecule; and
(g) identifying the at least one member of the peptide library that interacts with the target molecule by the sequence of the nucleic acid molecule from which it was expressed.

2-69. (canceled)


SEQUENCE LISTING (69 sequences)

SEQ ID NO: 1 - DNA, 1719 bp, Artificial Sequence
  tac-Ck-repA-CIS-ori sequence
cggcggttagaacgcggctacaattaatacataaccccatccccctgttgacaattaatcatcggctcgtataatgtgtggaattgtgagcggataacaatttcacacaggaaacaggatctaccatggccggatctaccatggcccagatacgcgccactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggaggcagcggttctagtctagcggccccaactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgtgccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttaagtcccgtggaataaaacgtgcccgtgcgcgtcgtgatgcgaacagagaacgtcaggatatcgtcaccctggtgaaacggcagctgacgcgcgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 2 - DNA, 27 bp, Artificial Sequence
  S-R1RecFor primer
gaacgcggctacaattaatacataacc

SEQ ID NO: 3 - DNA, 41 bp, Artificial Sequence
  #514 ThioBioXho85 primer
ggtgatcagtcagctcgagtgcatatctgtctgtccacagg

SEQ ID NO: 4 - DNA, 1729 bp, Artificial Sequence
  tac-Ck-repA-CIS-ori-bio sequence
gaacgcggctacaattaatacataaccccatccccctgttgacaattaatcatcggctcgtataatgtgtggaattgtgagcggataacaatttcacacaggaaacaggatctaccatggccggatctaccatggcccagatacgcgccactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggaggcagcggttctagtctagcggccccaactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgtgccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttaagtcccgtggaataaaacgtgcccgtgcgcgtcgtgatgcgaacagagaacgtcaggatatcgtcaccctggtgaaacggcagctgacgcgcgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcactcgagctgactgatcacc

SEQ ID NO: 5 - DNA, 1391 bp, Artificial Sequence
  tac-V5-repA-CIS-ori sequence
ccccatccccctgttgacaattaatcatcggctcgtataatgtgtggaattgtgagcggataacaatttcacacaggaaacaggatctaccatggccgcaggaaaacctatcccaaaccctctcctaggactggattcaacgggcagcggttctagtctagcggccccaactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgtgccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttaagtcccgtggaataaaacgtgcccgtgcgcgtcgtgatgcgaacagagaacgtcaggatatcgtcaccctggtgaaacggcagctgacgcgcgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 6 - DNA, 1410 bp, Artificial Sequence
  tac-V5-repA-CIS-ori-bio sequence
ccccatccccctgttgacaattaatcatcggctcgtataatgtgtggaattgtgagcggataacaatttcacacaggaaacaggatctaccatggccgcaggaaaacctatcccaaaccctctcctaggactggattcaacgggcagcggttctagtctagcggccccaactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgtgccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttaagtcccgtggaataaaacgtgcccgtgcgcgtcgtgatgcgaacagagaacgtcaggatatcgtcaccctggtgaaacggcagctgacgcgcgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcactcgagctgactgatcacc

SEQ ID NO: 7 - DNA, 1416 bp, Artificial Sequence
  bio-tac-V5-repA-CIS-ori sequence
gaacgcggctacaattaatacataaccccatccccctgttgacaattaatcatcggctcgtataatgtgtggaattgtgagcggataacaatttcacacaggaaacaggatctaccatggccgcaggaaaacctatcccaaaccctctcctaggactggattcaacgggcagcggttctagtctagcggccccaactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgtgccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttaagtcccgtggaataaaacgtgcccgtgcgcgtcgtgatgcgaacagagaacgtcaggatatcgtcaccctggtgaaacggcagctgacgcgcgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 8 - DNA, 26 bp, Artificial Sequence
  #144 tac6 primer
ccccatccccctgttgacaattaatc

SEQ ID NO: 9 - DNA, 27 bp, Artificial Sequence
  #472 R1RecForbio primer
gaacgcggctacaattaatacataacc

SEQ ID NO: 10 - DNA, 22 bp, Artificial Sequence
  #85 Orirev primer
tgcatatctgtctgtccacagg

SEQ ID NO: 11 - DNA, 1249 bp, Artificial Sequence
  1steprepA sequence
ggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 12 - DNA, 69 bp, Artificial Sequence
  flag-libfor primer
ggaaacaggatctaccatggcccagnasnasnasnasnasnasnasnasggcagcggttctagtctagc

SEQ ID NO: 13 - DNA, 1298 bp, Artificial Sequence
  flaglib-repA-CIS-ori sequence
ggaaacaggatctaccatggcccagnasnasnasnasnasnasnasnasggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 14 - DNA, 131 bp, Artificial Sequence
  131-mer primer
cggcggttagaacgcggctacaattaatacataaccccatccccctgttgacaattaatcatcggctcgtataatgtgtggaattgtgagcggataacaatttcacacaggaaacaggatctaccatggcc

SEQ ID NO: 15 - DNA, 1407 bp, Artificial Sequence
  tac-flaglib-repA-CIS-ori sequence
cggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 16 - DNA, 92 bp, Artificial Sequence
  Primer A
aatgatacggcgaccaccgagatctacactctttccctacacgacgctcttccgatctcgtaggtctcagttggggccgctagactagaacc

SEQ ID NO: 17 - DNA, 55 bp, Artificial Sequence
  Primer B
caagcagaagacggcatacgagctcttccgatctcggcggttagaacgcggctac

SEQ ID NO: 18 - DNA, 92 bp, Artificial Sequence; identical to SEQ ID NO: 16
  Primer C

SEQ ID NO: 19 - DNA, 82 bp, Artificial Sequence
  Primer D
caagcagaagacggcatacgagatccgtctcggcattcctgctgaaccgctcttccgatctcggcggttagaacgcggctac

SEQ ID NO: 20 - DNA, 39 bp, Artificial Sequence
  Primer C′
ttttttttttaatgatacggcgaccaccgaganctacac

SEQ ID NO: 21 - DNA, 34 bp, Artificial Sequence
  Primer D′
ttttttttttcaagcagaagacggcatacgagat

SEQ ID NO: 22 - DNA, 33 bp, Artificial Sequence
  Read 1 specific sequencing primer
acactctttccctacacgacgctcttccgatct

SEQ ID NO: 23 - DNA, 37 bp, Artificial Sequence
  Bsa repfor primer
aaaggtctcccaactgatcttcaccaaacgtattacc

SEQ ID NO: 24 - DNA, 81 bp, Artificial Sequence
  Primer E
aatgatacggcgaccaccgagatctacactctttccctacacgacgctcttccgatctctgcatatctgtctgtccacagg

SEQ ID NO: 25 - DNA, 65 bp, Artificial Sequence
  Adapter A
ccatctcatccctgcgtgtcccatctgttccctccctgtctcagcggcggttagaacgcggctac

SEQ ID NO: 26 - DNA, 66 bp, Artificial Sequence
  Adapter B
cctatcccctgtgtgccttgcctatcccctgttgcgtgtctcagtgcatatctgtctgtccacagg

SEQ ID NO: 27 - DNA, 1495 bp, Artificial Sequence
  tac-flaglib-repA-CIS-ori-454adapt sequence
ccatctcatccctgcgtgtcccatctgttccctccctgtctcagcggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcactgagacacgcaacaggggataggcaaggcacacaggggatagg

SEQ ID NO: 28 - DNA, 20 bp, Artificial Sequence
  HEG capture primer
cctatcccctgtgtgccttg

SEQ ID NO: 29 - DNA, 20 bp, Artificial Sequence
  454 Seq Forward primer
ccatctcatccctgcgtgtc

SEQ ID NO: 30 - DNA, 20 bp, Artificial Sequence
  454 Seq Reverse primer
cctatcccctgtgtgccttg

SEQ ID NO: 31 - DNA, 40 bp, Artificial Sequence
  HEG enrichment primer
ccatctcatccctgcgtgtcccatctgttccctccctgtc

SEQ ID NO: 32 - DNA, 51 bp, Artificial Sequence
  Primer A-key
ccatctcatccctgcgtgtctccgactcagcggcggttagaacgcggctac

SEQ ID NO: 33 - DNA, 45 bp, Artificial Sequence
  Primer P1-key
cctctctatgggcagtcggtgattgcatatctgtctgtccacagg

SEQ ID NO: 34 - DNA, 69 bp, Artificial Sequence
  6mer-libfor primer
ggaaacaggatctaccatggcccagnnsnnsnnsnnsnnsnnsnnsnnsggcagcggttctagtctagc

SEQ ID NO: 35 - DNA, 150 bp, Artificial Sequence
  Pinlibfor primer
ggaaacaggatctaccatggccgatgaagagaaactgccgccaggctggnnbaaannbtggagtvvmvvmggacgcgtcnnbtacnnbaatnnbatcactnnbgcgvvmcagtgggaacgaccatcgggcggcagcggttctagtctagc

SEQ ID NO: 36 - DNA, 34 bp, Artificial Sequence; identical to SEQ ID NO: 21
  Oligo D2 primer

SEQ ID NO: 37 - DNA, 22 bp, Artificial Sequence
  OrirevAlex647 primer
tgcatatctgtctgtccacagg

SEQ ID NO: 38 - DNA, 317 bp, Artificial Sequence
  tac-flaglib-illmunadapt sequence
caagcagaagacggcatacgagatccgtctcggcattcctgctgaaccgctcttccgatctcggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasggcagcggttctagtctagcggccccaactgagacctacgagatcggaagagcgtcgtgtagggaaagagtgtagatctcggtggtcgccgtatcatt

SEQ ID NO: 39 - DNA, 1234 bp, Artificial Sequence
  bsarepA-CIS-ori sequence
aaaggtctcccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 40 - DNA, 1527 bp, Artificial Sequence
  tac-flaglib-repA-CIS-ori-illumadapt sequence
caagcagaagacggcatacgagatccgtctcggcattcctgctgaaccgctcttccgatctcggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcagagatcggaagagcgtcgtgtagggaaagagtgtagatctcggtggtcgccgtatcatt

SEQ ID NO: 41 - DNA, 1460 bp, Artificial Sequence
  tac-flaglib-repA-CIS-ori-ionadapt sequence
ccatctcatccctgcgtgtctccgactcagcggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcaatcaccgactgcccatagagagg

SEQ ID NO: 42 - DNA, 1407 bp, Artificial Sequence; identical to SEQ ID NO: 15
  tac-6merlib-repA-CIS-ori sequence

SEQ ID NO: 43 - DNA, 1521 bp, Artificial Sequence
  tac-6merlib-repA-CIS-ori-illumadapt sequence
caagcagaagacggcatacgagatccgtctcggcattcctgctgaaccgctcttccgatctcggcggttagaacgcggctacaattaatacataaccccatccccctgttgacaattaatcatcggctcgtataatgtgtggaattgtgagcggataacaatttcacacaggaaacaggatctaccatggcccagnnknnknnknnknnknnkggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcagagatcggaagagcgtcgtgtagggaaagagtgtagatctcggtggtcgccgtatcatt

SEQ ID NO: 44 - DNA, 1495 bp, Artificial Sequence; identical to SEQ ID NO: 27
  tac-6merlib-repA-CIS-ori-454adapt sequence

SEQ ID NO: 45 - DNA, 1488 bp, Artificial Sequence
  tac-pinlib-repA-CIS-ori sequence
cggcggttagaacgcggctacaattaatacataaccccatccccctgttgacaattaatcatcggctcgtataatgtgtggaattgtgagcggataacaatttcacacaggaaacaggatctaccatggccgatgaagagaaactgccgccaggctggnnbaaannbtggagtvvmvvmggacgcgtcnnbtacnnbaatnnbatcactnnbgcgvvmcagtgggaacgaccatcgggcggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 46 - DNA, 26 bp, Artificial Sequence
  Pinlibfor primer
gccgatgaagagaaactgccgccagg

SEQ ID NO: 47 - DNA, 20 bp, Artificial Sequence
  Pinlibrev primer
cccgatggtcgttcccactg

SEQ ID NO: 48 - DNA, 3116 bp, Artificial Sequence
  tacP2AHA sequence
gcttcagtaagccagatgctacacaattaggcttgtacatattgtcgttagaacgcggctacaattaatacataaccttatgtatcatacacatacgatttaggtgacactatagaatacaagcttactccccatccccctgttgacaattaatcatggctcgtataatgtgtggaattgtgagcggataacaatttcacacaggaaacaggatctaccatggccgttaaagcctccgggcgttttgtccctccgtcagcatttgccgcaggcaccggtaagatgtttaccggtgcttatgcatggaacgcgccacggcaggccgtcgggcgcgaaagaccccttacacgtgacgagatgcgtcagatgcaaggtgttttatccacgattaaccgcctgccttactttttgcgctcgctgtttacttcacgctatgactacatccggcgcaataaaagcccggtgcacgggttttatttcctcacatccacttttcagcgtcgtttatggccgcgcattgagcgtgtgaatcagcgccatgaaatgaacaccgacgcgtcgttgctgtttctggcagagcgtgaccactatgcgcgcctgccgggaatgaatgacaaggagctgaaaaagtttgccgcccgtatctcatcgcagcttttcatgatgtatgaggaactcagcgatgcctgggtggatgcacatggcgaaaaagaatcgctgtttacggatgaggcgcaggctcacctctatggtcatgttgctggcgctgcacgtgctttcaatatttccccgctttactggaaaaaataccgtaaaggacagatgaccacgaggcaggcatattctgccattgcccgtctgtttaacgatgagtggtggactcatcagctcaaaggccagcgtatgcgctggcatgaggcgttactgattgctgtcggggaggtgaataaagaccgttctccttatgccagtaaacatgccattcgtgatgtgcgtgcacgccgccaagcaaatctggaatttcttaaatcgtgtgaccttgaaaacagggaaaccggcgagcgcatcgaccttatcagtaaggtgatgggcagtatttctaatcctgaaattcgccggatggagctgatgaacaccattgccggtattgagcgttacgccgccgcagagggtgatgtggggatgtttatcacgcttaccgcgccttcaaagtatcacccgacacgtcaggtcggaaaaggcgaaagtaaaaccgtccagctaaatcacggctggaacgatgaggcatttaatccaaaggatgcgcagcgttatctctgccatatctggagcctgatgcgcacggcattcaaagataatgatttacaggtctacggtttgcgtgtcgtcgagccacaccacgacggaacgccgcactggcatatgatgcttttttgtaatccacgccagcgtaaccagattatcgaaatcatgcgtcgctatgcgctcaaagaggatggcgacgaaagaggagccgcgcgaaaccgttttcaggcaaaacaccttaaccagggcggtgctgcggggtatatcgcgaaatacatctcaaaaaacatcgatggctatgcactggatggtcagctcgataacgataccggcagaccgctgaaagacactgctgcggctgttaccgcatgggcgtcaacgtggcgcatcccacaatttaaaacggttggtctgccgacaatgggggcttaccgtgaactacgcaaattgcctcgcggcgtcagcattgctgatgagtttgacgagcgcgtcgaggctgcacgcgccgccgcagacagtggtgattttgcgttgtatatcagcgcgcagggtggggcaaatgtcccgcgcgattgtcagactgtcagggtcgcccgtagtccgtcggatgaggttaacgagtacgaggaagaagtcgagagagtggtcggcatttacgcgccgcatctcggcgcgcgtcatattcatatcaccagaacgacggactggcgcattgtgccgaaagttccggtcgttgagcctctgactttaaaaagcggcatcgccgcgcctcggagtcctgtcaataactgtggaaagctcaccggtggtgatacttcgttaccggctcccacaccttctgagcacgccgcagcagtgcttaatctggttgatgacggtgttattgaatggaatgaaccggaggtcgtgagggcgctcaggggcgcattaaaatacgacatgagaacgccaaaccgtcagcaaagaaacggaagcccgttaaaaccgcatgaaattgcaccatctgccagactgaccaggtctgaacgattgcagatcacccgtatccgcgttgaccttgctcagaacggtatcaggcctcagcgatgggaacttgaggcgctggcgcgtggagcaaccgtaaattatgacgggaaaaaattcacgtatccggtcgctgatgagtggccgggattctcaacagtaatggagtggacactcgagatggcttacccgtacgacgttccggactacgctcgttgatagaattcatcgagcccgcctaatgagcgggcttttttttcgatgatatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgta

SEQ ID NO: 49 - DNA, 22 bp, Artificial Sequence
  LAMPB primer
tacaccgaactgagatacctac

SEQ ID NO: 50 - DNA, 25 bp, Artificial Sequence
  LinkP2Afor primer
gttaaagcctccgggcgttttgtcc

SEQ ID NO: 51 - DNA, 22 bp, Artificial Sequence
  P2AAmpF primer
gcttcagtaagccagatgctac

SEQ ID NO: 52 - DNA, 2891 bp, Artificial Sequence
  Link-P2A sequence
gttaaagcctccgggcgttttgtccctccgtcagcatttgccgcaggcaccggtaagatgtttaccggtgcttatgcatggaacgcgccacggcaggccgtcgggcgcgaaagaccccttacacgtgacgagatgcgtcagatgcaaggtgttttatccacgattaaccgcctgccttactttttgcgctcgctgtttacttcacgctatgactacatccggcgcaataaaagcccggtgcacgggttttatttcctcacatccacttttcagcgtcgtttatggccgcgcattgagcgtgtgaatcagcgccatgaaatgaacaccgacgcgtcgttgctgtttctggcagagcgtgaccactatgcgcgcctgccgggaatgaatgacaaggagctgaaaaagtttgccgcccgtatctcatcgcagcttttcatgatgtatgaggaactcagcgatgcctgggtggatgcacatggcgaaaaagaatcgctgtttacggatgaggcgcaggctcacctctatggtcatgttgctggcgctgcacgtgctttcaatatttccccgctttactggaaaaaataccgtaaaggacagatgaccacgaggcaggcatattctgccattgcccgtctgtttaacgatgagtggtggactcatcagctcaaaggccagcgtatgcgctggcatgaggcgttactgattgctgtcggggaggtgaataaagaccgttctccttatgccagtaaacatgccattcgtgatgtgcgtgcacgccgccaagcaaatctggaatttcttaaatcgtgtgaccttgaaaacagggaaaccggcgagcgcatcgaccttatcagtaaggtgatgggcagtatttctaatcctgaaattcgccggatggagctgatgaacaccattgccggtattgagcgttacgccgccgcagagggtgatgtggggatgtttatcacgcttaccgcgccttcaaagtatcacccgacacgtcaggtcggaaaaggcgaaagtaaaaccgtccagctaaatcacggctggaacgatgaggcatttaatccaaaggatgcgcagcgttatctctgccatatctggagcctgatgcgcacggcattcaaagataatgatttacaggtctacggtttgcgtgtcgtcgagccacaccacgacggaacgccgcactggcatatgatgcttttttgtaatccacgccagcgtaaccagattatcgaaatcatgcgtcgctatgcgctcaaagaggatggcgacgaaagaggagccgcgcgaaaccgttttcaggcaaaacaccttaaccagggcggtgctgcggggtatatcgcgaaatacatctcaaaaaacatcgatggctatgcactggatggtcagctcgataacgataccggcagaccgctgaaagacactgctgcggctgttaccgcatgggcgtcaacgtggcgcatcccacaatttaaaacggttggtctgccgacaatgggggcttaccgtgaactacgcaaattgcctcgcggcgtcagcattgctgatgagtttgacgagcgcgtcgaggctgcacgcgccgccgcagacagtggtgattttgcgttgtatatcagcgcgcagggtggggcaaatgtcccgcgcgattgtcagactgtcagggtcgcccgtagtccgtcggatgaggttaacgagtacgaggaagaagtcgagagagtggtcggcatttacgcgccgcatctcggcgcgcgtcatattcatatcaccagaacgacggactggcgcattgtgccgaaagttccggtcgttgagcctctgactttaaaaagcggcatcgccgcgcctcggagtcctgtcaataactgtggaaagctcaccggtggtgatacttcgttaccggctcccacaccttctgagcacgccgcagcagtgcttaatctggttgatgacggtgttattgaatggaatgaaccggaggtcgtgagggcgctcaggggcgcattaaaatacgacatgagaacgccaaaccgtcagcaaagaaacggaagcccgttaaaaccgcatgaaattgcaccatctgccagactgaccaggtctgaacgattgcagatcacccgtatccgcgttgaccttgctcagaacggtatcaggcctcagcgatgggaacttgaggcgctggcgcgtggagcaaccgtaaattatgacgggaaaaaattcacgtatccggtcgctgatgagtggccgggattctcaacagtaatggagtggacactcgagatggcttacccgtacgacgttccggactacgctcgttgatagaattcatcgagcccgcctaatgagcgggcttttttttcgatgatatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgta

SEQ ID NO: 53 - DNA, 78 bp, Artificial Sequence
  flaglib-p2afor primer
ggaaacaggatctaccatggcccagnasnasnasnasnasnasnasnasgttaaagcctccgggcgttttgtccctcc

SEQ ID NO: 54 - DNA, 2940 bp, Artificial Sequence
  flaglib-P2A sequence
ggaaacaggatctaccatggcccagnasnasnasnasnasnasnasnasgttaaagcctccgggcgttttgtccctccgtcagcatttgccgcaggcaccggtaagatgtttaccggtgcttatgcatggaacgcgccacggcaggccgtcgggcgcgaaagaccccttacacgtgacgagatgcgtcagatgcaaggtgttttatccacgattaaccgcctgccttactttttgcgctcgctgtttacttcacgctatgactacatccggcgcaataaaagcccggtgcacgggttttatttcctcacatccacttttcagcgtcgtttatggccgcgcattgagcgtgtgaatcagcgccatgaaatgaacaccgacgcgtcgttgctgtttctggcagagcgtgaccactatgcgcgcctgccgggaatgaatgacaaggagctgaaaaagtttgccgcccgtatctcatcgcagcttttcatgatgtatgaggaactcagcgatgcctgggtggatgcacatggcgaaaaagaatcgctgtttacggatgaggcgcaggctcacctctatggtcatgttgctggcgctgcacgtgctttcaatatttccccgctttactggaaaaaataccgtaaaggacagatgaccacgaggcaggcatattctgccattgcccgtctgtttaacgatgagtggtggactcatcagctcaaaggccagcgtatgcgctggcatgaggcgttactgattgctgtcggggaggtgaataaagaccgttctccttatgccagtaaacatgccattcgtgatgtgcgtgcacgccgccaagcaaatctggaatttcttaaatcgtgtgaccttgaaaacagggaaaccggcgagcgcatcgaccttatcagtaaggtgatgggcagtatttctaatcctgaaattcgccggatggagctgatgaacaccattgccggtattgagcgttacgccgccgcagagggtgatgtggggatgtttatcacgcttaccgcgccttcaaagtatcacccgacacgtcaggtcggaaaaggcgaaagtaaaaccgtccagctaaatcacggctggaacgatgaggcatttaatccaaaggatgcgcagcgttatctctgccatatctggagcctgatgcgcacggcattcaaagataatgatttacaggtctacggtttgcgtgtcgtcgagccacaccacgacggaacgccgcactggcatatgatgcttttttgtaatccacgccagcgtaaccagattatcgaaatcatgcgtcgctatgcgctcaaagaggatggcgacgaaagaggagccgcgcgaaaccgttttcaggcaaaacaccttaaccagggcggtgctgcggggtatatcgcgaaatacatctcaaaaaacatcgatggctatgcactggatggtcagctcgataacgataccggcagaccgctgaaagacactgctgcggctgttaccgcatgggcgtcaacgtggcgcatcccacaatttaaaacggttggtctgccgacaatgggggcttaccgtgaactacgcaaattgcctcgcggcgtcagcattgctgatgagtttgacgagcgcgtcgaggctgcacgcgccgccgcagacagtggtgattttgcgttgtatatcagcgcgcagggtggggcaaatgtcccgcgcgattgtcagactgtcagggtcgcccgtagtccgtcggatgaggttaacgagtacgaggaagaagtcgagagagtggtcggcatttacgcgccgcatctcggcgcgcgtcatattcatatcaccagaacgacggactggcgcattgtgccgaaagttccggtcgttgagcctctgactttaaaaagcggcatcgccgcgcctcggagtcctgtcaataactgtggaaagctcaccggtggtgatacttcgttaccggctcccacaccttctgagcacgccgcagcagtgcttaatctggttgatgacggtgttattgaatggaatgaaccggaggtcgtgagggcgctcaggggcgcattaaaatacgacatgagaacgccaaaccgtcagcaaagaaacggaagcccgttaaaaccgcatgaaattgcaccatctgccagactgaccaggtctgaacgattgcagatcacccgtatccgcgttgaccttgctcagaacggtatcaggcctcagcgatgggaacttgaggcgctggcgcgtggagcaaccgtaaattatgacgggaaaaaattcacgtatccggtcgctgatgagtggccgggattctcaacagtaatggagtggacactcgagatggcttacccgtacgacgttccggactacgctcgttgatagaattcatcgagcccgcctaatgagcgggcttttttttcgatgatatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgta

SEQ ID NO: 55 - DNA, 3049 bp, Artificial Sequence
  tacflaglib-P2A sequence
cggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasgttaaagcctccgggcgttttgtccctccgtcagcatttgccgcaggcaccggtaagatgtttaccggtgcttatgcatggaacgcgccacggcaggccgtcgggcgcgaaagaccccttacacgtgacgagatgcgtcagatgcaaggtgttttatccacgattaaccgcctgccttactttttgcgctcgctgtttacttcacgctatgactacatccggcgcaataaaagcccggtgcacgggttttatttcctcacatccacttttcagcgtcgtttatggccgcgcattgagcgtgtgaatcagcgccatgaaatgaacaccgacgcgtcgttgctgtttctggcagagcgtgaccactatgcgcgcctgccgggaatgaatgacaaggagctgaaaaagtttgccgcccgtatctcatcgcagcttttcatgatgtatgaggaactcagcgatgcctgggtggatgcacatggcgaaaaagaatcgctgtttacggatgaggcgcaggctcacctctatggtcatgttgctggcgctgcacgtgctttcaatatttccccgctttactggaaaaaataccgtaaaggacagatgaccacgaggcaggcatattctgccattgcccgtctgtttaacgatgagtggtggactcatcagctcaaaggccagcgtatgcgctggcatgaggcgttactgattgctgtcggggaggtgaataaagaccgttctccttatgccagtaaacatgccattcgtgatgtgcgtgcacgccgccaagcaaatctggaatttcttaaatcgtgtgaccttgaaaacagggaaaccggcgagcgcatcgaccttatcagtaaggtgatgggcagtatttctaatcctgaaattcgccggatggagctgatgaacaccattgccggtattgagcgttacgccgccgcagagggtgatgtggggatgtttatcacgcttaccgcgccttcaaagtatcacccgacacgtcaggtcggaaaaggcgaaagtaaaaccgtccagctaaatcacggctggaacgatgaggcatttaatccaaaggatgcgcagcgttatctctgccatatctggagcctgatgcgcacggcattcaaagataatgatttacaggtctacggtttgcgtgtcgtcgagccacaccacgacggaacgccgcactggcatatgatgcttttttgtaatccacgccagcgtaaccagattatcgaaatcatgcgtcgctatgcgctcaaagaggatggcgacgaaagaggagccgcgcgaaaccgttttcaggcaaaacaccttaaccagggcggtgctgcggggtatatcgcgaaatacatctcaaaaaacatcgatggctatgcactggatggtcagctcgataacgataccggcagaccgctgaaagacactgctgcggctgttaccgcatgggcgtcaacgtggcgcatcccacaatttaaaacggttggtctgccgacaatgggggcttaccgtgaactacgcaaattgcctcgcggcgtcagcattgctgatgagtttgacgagcgcgtcgaggctgcacgcgccgccgcagacagtggtgattttgcgttgtatatcagcgcgcagggtggggcaaatgtcccgcgcgattgtcagactgtcagggtcgcccgtagtccgtcggatgaggttaacgagtacgaggaagaagtcgagagagtggtcggcatttacgcgccgcatctcggcgcgcgtcatattcatatcaccagaacgacggactggcgcattgtgccgaaagttccggtcgttgagcctctgactttaaaaagcggcatcgccgcgcctcggagtcctgtcaataactgtggaaagctcaccggtggtgatacttcgttaccggctcccacaccttctgagcacgccgcagcagtgcttaatctggttgatgacggtgttattgaatggaatgaaccggaggtcgtgagggcgctcaggggcgcattaaaatacgacatgagaacgccaaaccgtcagcaaagaaacggaagcccgttaaaaccgcatgaaattgcaccatctgccagactgaccaggtctgaacgattgcagatcacccgtatccgcgttgaccttgctcagaacggtatcaggcctcagcgatgggaacttgaggcgctggcgcgtggagcaaccgtaaattatgacgggaaaaaattcacgtatccggtcgctgatgagtggccgggattctcaacagtaatggagtggacactcgagatggcttacccgtacgacgttccggactacgctcgttgatagaattcatcgagcccgcctaatgagcgggcttttttttcgatgatatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgta

SEQ ID NO: 56 - DNA, 71 bp, Artificial Sequence
  Adapter C primer
cctatcccctgtgtgccttgcctatcccctgttgcgtgtctcatacaccgaactgagatacctacagcgtg

SEQ ID NO: 57 - DNA, 3136 bp, Artificial Sequence
  tac-flaglib-P2A-454-adapted sequence
ccatctcatccctgcgtgtcccatctgttccctccctgtctcagcggcggttagaacgcggctacaattaatacataaccccatccccctgttgacaattaatcatcggctcgtataatgtgtggaattgtgagcggataacaatttcacacaggaaacaggatctaccatggcccagnasnasnasnasnasnasnasnasgttaaagcctccgggcgttttgtccctccgtcagcatttgccgcaggcaccggtaagatgtttaccggtgcttatgcatggaacgcgccacggcaggccgtcgggcgcgaaagaccccttacacgtgacgagatgcgtcagatgcaaggtgttttatccacgattaaccgcctgccttactttttgcgctcgctgtttacttcacgctatgactacatccggcgcaataaaagcccggtgcacgggttttatttcctcacatccacttttcagcgtcgtttatggccgcgcattgagcgtgtgaatcagcgccatgaaatgaacaccgacgcgtcgttgctgtttctggcagagcgtgaccactatgcgcgcctgccgggaatgaatgacaaggagctgaaaaagtttgccgcccgtatctcatcgcagcttttcatgatgtatgaggaactcagcgatgcctgggtggatgcacatggcgaaaaagaatcgctgtttacggatgaggcgcaggctcacctctatggtcatgttgctggcgctgcacgtgctttcaatatttccccgctttactggaaaaaataccgtaaaggacagatgaccacgaggcaggcatattctgccattgcccgtctgtttaacgatgagtggtggactcatcagctcaaaggccagcgtatgcgctggcatgaggcgttactgattgctgtcggggaggtgaataaagaccgttctccttatgccagtaaacatgccattcgtgatgtgcgtgcacgccgccaagcaaatctggaatttcttaaatcgtgtgaccttgaaaacagggaaaccggcgagcgcatcgaccttatcagtaaggtgatgggcagtatttctaatcctgaaattcgccggatggagctgatgaacaccattgccggtattgagcgttacgccgccgcagagggtgatgtggggatgtttatcacgcttaccgcgccttcaaagtatcacccgacacgtcaggtcggaaaaggcgaaagtaaaaccgtccagctaaatcacggctggaacgatgaggcatttaatccaaaggatgcgcagcgttatctctgccatatctggagcctgatgcgcacggcattcaaagataatgatttacaggtctacggtttgcgtgtcgtcgagccacaccacgacggaacgccgcactggcatatgatgcttttttgtaatccacgccagcgtaaccagattatcgaaatcatgcgtcgctatgcgctcaaagaggatggcgacgaaagaggagccgcgcgaaaccgttttcaggcaaaacaccttaaccagggcggtgctgcggggtatatcgcgaaatacatctcaaaaaacatcgatggctatgcactggatggtcagctcgataacgataccggcagaccgctgaaagacactgctgcggctgttaccgcatgggcgtcaacgtggcgcatcccacaatttaaaacggttggtctgccgacaatgggggcttaccgtgaactacgcaaattgcctcgcggcgtcagcattgctgatgagtttgacgagcgcgtcgaggctgcacgcgccgccgcagacagtggtgattttgcgttgtatatcagcgcgcagggtggggcaaatgtcccgcgcgattgtcagactgtcagggtcgcccgtagtccgtcggatgaggttaacgagtacgaggaagaagtcgagagagtggtcggcatttacgcgccgcatctcggcgcgcgtcatattcatatcaccagaacgacggactggcgcattgtgccgaaagttccggtcgttgagcctctgactttaaaaagcggcatcgccgcgcctcggagtcctgtcaataactgtggaaagctcaccggtggtgatacttcgttaccggctcccacaccttctgagcacgccgcagcagtgcttaatctggttgatgacggtgttattgaatggaatgaaccggaggtcgtgagggcgctcaggggcgcattaaaatacgacatgagaacgccaaaccgtcagcaaagaaacggaagcccgttaaaaccgcatgaaattgcaccatctgccagactgaccaggtctgaacgattgcagatcacccgtatccgcgttgaccttgctcagaacggtatcaggcctcagcgatgggaacttgaggcgctggcgcgtggagcaaccgtaaattatgacgggaaaaaattcacgtatccggtcgctgatgagtggccgggattctcaacagtaatggagtggacactcgagatggcttacccgtacgacgttccggactacgctcgttgatagaattcatcgagcccgcctaatgagcgggcttttttttcgatgatatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtatgagacacgcaacaggggataggcaaggcacacaggggatagg

SEQ ID NO: 58 - DNA, 118 bp, Artificial Sequence
  R1-ori sequence
ttatccacatttaactgcaagggacttccccataaggttacaaccgttcatgtcataaagcgccagccgccagtcttacagggtgcaatgtatcttttaaacacctgtttatatctcc

SEQ ID NO: 59 - DNA, 118 bp, Artificial Sequence
  R100-ori sequence
ttatccacattaaactgcaagggacttccccataaggttacaaccgttcatgtcataaagcgccatccgccagcgttacagggtgcaatgtatcttttaaacacctgtttatatctcc

SEQ ID NO: 60 - DNA, 20 bp, Artificial Sequence
  P2A ori sequence
gcgcctcggagtcctgtcaa

SEQ ID NO: 61 - protein, 5 aa, Artificial Sequence
  Amino acid linker
GSGSS

SEQ ID NO: 62 - DNA, 90 bp, Artificial Sequence
  15mer-lib1for primer
ggaaacaggatctaccatggcccagyacscgatsracracytgytgracyacsttsttscgaramtgcrtggcagcggttctagtctagc

SEQ ID NO: 63 - DNA, 139 bp, Artificial Sequence
  15mer-lib2for primer
ggaaacaggatctaccatggccgatgaagagaaactgccgccaggctggscggyacscgatsracracytgytgracyacsttsttscgaramtgcrtcagtgggaacgaccatcgggcggcagcggttctagtctagc

SEQ ID NO: 64 - DNA, 1428 bp, Artificial Sequence
  tac-15merlib1-repA-CIS-ori sequence
cggcggttagaacgcggctacaattaatacataaccccatccccctgttgacaattaatcatcggctcgtataatgtgtggaattgtgagcggataacaatttcacacaggaaacaggatctaccatggcccagyacscgatsracracytgytgracyacsttsttscgaramtgcrtggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 65 - DNA, 1548 bp, Artificial Sequence
  tac-15merlib1-repA-CIS-ori-illumadapt sequence
caagcagaagacggcatacgagatccgtctcggcattcctgctgaaccgctcttccgatctcggcggttagaacgcggctacaattaatacataaccccatccccctgttgacaattaatcatcggctcgtataatgtgtggaattgtgagcggataacaatttcacacaggaaacaggatctaccatggcccagyacscgatsracracytgytgracyacsttsttscgaramtgcrtggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcagagatcggaagagcgtcgtgtagggaaagagtgtagatctcggtggtcgccgtatcatt

SEQ ID NO: 66 - DNA, 1516 bp, Artificial Sequence
  tac-15merlib1-repA-CIS-ori-454adapt sequence
ccatctcatccctgcgtgtcccatctgttccctccctgtctcagcggcggttagaacgcggctacaattaatacataaccccatccccctgttgacaattaatcatcggctcgtataatgtgtggaattgtgagcggataacaatttcacacaggaaacaggatctaccatggcccagyacscgatsracracytgytgracyacsttsttscgaramtgcrtggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcactgagacacgcaacaggggataggcaaggcacacaggggatagg

SEQ ID NO: 67 - DNA, 1473 bp, Artificial Sequence
  tac-15merlib2-repA-CIS-ori sequence
cggcggttagaacgcggctacaattaatacataaccccatccccctgttgacaattaatcatcggctcgtataatgtgtggaattgtgagcggataacaatttcacacaggaaacaggatctaccatggccgatgaagagaaactgccgccaggctggyacscgatsracracytgytgracyacsttsttscgaramtgcrtcagtgggaacgaccatcgggcggcagcggttctagtctagcggccccaactgatcttcaccaaacgtattaccgccaggtaaagaacccgaatccggtgttcactccccgtgaaggtgccggaacgccgaagttccgcgaaaaaccgatggaaaaggcggtgggcctcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 68 - DNA, 26 bp, Artificial Sequence
  15merlib2-recoveryfor primer
gccgatgaagagaaactgccgccagg

SEQ ID NO: 69 - DNA, 20 bp, Artificial Sequence
  15merlib2-recoveryrev primer
cccgatggtcgttcccactg